(19) United States
(12) Patent Application Publication
(10) Pub. No.: US 2023/0181612 A1
(43) Pub. Date: Jun. 15, 2023

(54) AMINOGLYCOSIDE DERIVATIVES AND USES THEREOF IN TREATING GENETIC DISORDERS

(71) Applicant: ELOXX PHARMACEUTICALS LTD., Rehovot (IL)

(72) Inventors: Timor BAASOV, Haifa (IL); Vera FIRZON, Haifa (IL); Valery BELAKHOV, Haifa (IL); Bat-Hen EYLON, Kfar-Saba (IL)

(21) Appl. No.: 16/619,445

(22) PCT Filed: Jun. 5, 2018
(86) PCT No.: PCT/IL2018/050612

§ 371 Date: Dec. 4, 2019

Related U.S. Application Data
(60) Provisional application No. 62/515,021, filed on Jun. 5, 2017.

(51) Int. Cl. A61K 31/7048; A61P 21/00

(52) U.S. Cl. CPC A61K 31/7048; A61P 21/00

(57) ABSTRACT

Novel aminoglycosides, represented by Formulae (e.g., a compound of Formula A, B, I, I*, III or III*, including compounds represented by Formula Ia, I**, I*a, I*b, IIIa, III**, III*a and, III*b), as defined in the instant specification, designed to exhibit stop codon mutation readthrough activity, are provided. Also provided are pharmaceutical compositions containing the same, and uses thereof in the treatment of genetic diseases and disorders, such as diseases and disorders associated with stop codon mutations.

### FIELD AND BACKGROUND OF THE INVENTION

The present invention, in some embodiments thereof, relates to aminoglycosides and more particularly, but not exclusively, to novel aminoglycoside derivatives and their use in increasing an expression of a gene having a stop codon mutation and/or in the treatment of genetic disorders.

Many human genetic disorders result from nonsense mutations, where one of the three stop codons (UAA, UAG or UGA) replaces an amino acid-coding codon, leading to premature termination of the translation and eventually to truncated inactive proteins. Currently, hundreds of such nonsense mutations are known, and several were shown to account for certain cases of fatal diseases, including, for example, cystic fibrosis (CF), nephropathic cystinosis, Duchenne muscular dystrophy (DMD), ataxia-telangiectasia, Hurler syndrome, hemophilia A, hemophilia B, Tay-Sachs, Rett Syndrome, Usher Syndrome, Severe epidermolysis bullosa and more. For many of those diseases there is presently no effective treatment.

Some aminoglycoside compounds have been shown to have therapeutic value in the treatment of several genetic diseases because of their ability to induce ribosomes to read-through stop codon mutations, generating full-length proteins from part of the mRNA molecules.

Aminoglycosides are highly potent, broad-spectrum antibiotics commonly used for the treatment of life-threatening infections. It is accepted that the mechanism of action of aminoglycoside antibiotics, such as paromomycin (see, FIG. 1), involves interaction with the prokaryotic ribosome, and, more specifically, involves binding to the decoding A-site of the 16S ribosomal RNA, which leads to protein translation inhibition and interference with the translational fidelity.

Several achievements in bacterial ribosome structure determination, along with crystal and NMR structures of bacterial A-site oligonucleotide models, have provided useful information for understanding the decoding mechanism in prokaryote cells and understanding how aminoglycosides exert their deleterious misreading of the genetic code. These studies and others have given rise to the hypothesis that the affinity of the A-site for a non-cognate mRNA-tRNA complex is increased upon aminoglycoside binding, preventing the ribosome from efficiently discriminating between non-cognate and cognate complexes.

The enhancement of termination suppression by aminoglycosides in eukaryotes is thought to occur in a similar mechanism to the aminoglycosides' activity in prokaryotes of interfering with translational fidelity during protein synthesis, namely the binding of certain aminoglycosides to the ribosomal A-site probably induce conformational changes that stabilize near-cognate mRNA-tRNA complexes, instead of inserting the release factor. Aminoglycosides have been shown to suppress various stop codons with notably different efficiencies (UGA>UAG>UAA), and the suppression effectiveness has been found to be further dependent upon the identity of the fourth nucleotide immediately downstream from the stop codon (C>U>A≥grams) as well as the local sequence context around the stop codon.

The desired characteristics of an effective read-through drug would be oral administration and little or no effect on bacteria. Antimicrobial activity of read-through drug is undesirable as any unnecessary use of antibiotics, particularly with respect to the gastrointestinal (GI) biota, due to the adverse effects caused by upsetting the GI biota equilibrium and the emergence of resistance. In this respect, in addition to the abovementioned limitations, the majority of clinical aminoglycosides are greatly selective against bacterial ribosomes, and do not exert a significant effect on cytoplasmic ribosomes of human cells.

In an effort to circumvent the abovementioned limitations, the biopharmaceutical industry is seeking new stop codon mutations suppression drugs by screening large chemical libraries for nonsense read-through activity.

The first experiments of aminoglycoside-mediated suppression of cystic fibrosis transmembrane conductance regulator protein (CFTR) stop codon mutations demonstrated that premature stop codon mutations found in the CFTR gene could be suppressed by members of the gentamicin family and geniticin® (G-418) (see, FIG. 1), as measured by the appearance of full-length, functional CFTR in human bronchial epithelial cell lines.

Suppression experiments of intestinal tissues from CFTR−/− transgenic mice mutants carrying a human CFTR-G542X transgene showed that treatment with gentamicin, and to lesser extent tobramycin, have resulted in the appearance of human CFTR protein at the glands of treated mice. Most importantly, clinical studies using double-blind, placebo-controlled, crossover trials have shown that gentamicin can suppress stop codon mutations in affected patients, and that gentamicin treatment improved transmembrane conductance across the nasal mucosa in a group of 19 patients carrying CFTR stop codon mutations. Other genetic disorders for which the therapeutic potential of aminoglycosides was tested in in-vitro systems, cultured cell lines, or animal models include DMD, Hurler syndrome, nephrogenic diabetes insipidus, nephropathic cystinosis, retinitis pigmentosa, and ataxia-telangiectasia.

However, one of the major limitations in using aminoglycosides as pharmaceuticals is their high toxicity towards mammals, typically expressed in kidney (nephrotoxicity) and ear-associated (ototoxicity) illnesses. The origin of this toxicity is assumed to result from a combination of different factors and mechanisms such as interactions with phospholipids, inhibition of phospholipases and the formation of free radicals.

Although considered selective to bacterial ribosomes, most aminoglycosides bind also to the eukaryotic A-site, but with lower affinities than to the bacterial A-site. The inhibition of translation in mammalian cells is also one of the possible causes for the high toxicity of these agents. Another factor adding to their cytotoxicity is their binding to the mitochondrial ribosome at the 12S rRNA A-site, whose sequence is very close to the bacterial A-site.

Many studies have been attempted to understand and offer ways to alleviate the toxicity associated with aminoglycosides, including the use of antioxidants to reduce free radical levels, as well as the use of poly-L-aspartate and daptomycin, to reduce the ability of aminoglycosides to interact with phospholipids. The role of megalin (a multiligand endocytic receptor which is especially abundant in the kidney proximal tubules and the inner ear) in the uptake of aminoglycosides has recently been demonstrated. The administration of agonists that compete for aminoglycoside binding to megalin also resulted in a reduction in aminoglycoside uptake and toxicity. In addition, altering the administration schedule and/or the manner in which aminoglycosides are administered has been investigated as means to reduce toxicity.

Despite extensive efforts to reduce aminoglycoside toxicity, few results have matured into standard clinical practices and procedures for the administration of aminoglycosides to suppress stop codon mutations, other than changes in the administration schedule. For example, the use of sub-toxic doses of gentamicin in the clinical trials probably caused the reduced read-through efficiency obtained in the in-vivo experiments compared to the in-vitro systems. The aminoglycoside geneticin® (also known as G-418 sulfate or simply G-418, as depicted below) showed the best termination suppression activity in in-vitro translation-transcription systems; however, its use as a therapeutic agent is not possible since it is lethal even at very low concentrations. For example, the LD₅₀ of G-418 against human fibroblast cells is 0.04 mg/ml, compared to 2.5-5.0 mg/ml for gentamicin, neomycin and kanamycin.

Since G-418 is extremely toxic even at very low concentrations, presently gentamicin is the only aminoglycoside tested in various animal models and clinical trials, while some studies have shown that amikacin and paromomycin can represent alternatives to gentamicin for stop codon mutation suppression therapy.

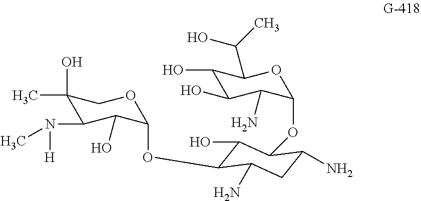 [SMILES: CNC1C(O)C(OC2C(N)CC(N)C(OC3OC(C(C)O)C(O)C(O)C3N)C2O)OCC1(C)O]

To date, nearly all suppression experiments have been performed with clinical, commercially available aminoglycosides, however, only a limited number of aminoglycosides, including gentamicin, amikacin, and tobramycin, are in clinical use as antibiotics for internal administration in humans. Among these, tobramycin do not have stop codon mutations suppression activity, and gentamicin is the only aminoglycoside tested for stop codon mutations suppression activity in animal models and clinical trials. Recently, a set of neamine derivatives were shown to promote read-through of the SMN protein in fibroblasts derived from spinal muscular atrophy (SPA) patients; however, these compounds were originally designed as antibiotics and no conclusions were derived for further improvement of the read-through activity of these derivatives.

WO 2007/113841 and WO 2012/066546 disclose classes of paromomycin-derived aminoglycosides, designed to exhibit high premature stop codon mutations readthrough activity while exerting low cytotoxicity in mammalian cells and low antimicrobial activity, and can thus be used in the treatment of genetic diseases. This class of paromomycin-derived aminoglycosides was designed by introducing certain manipulations to the paromamine core, which lead to enhanced readthrough activity and reduced toxicity and antimicrobial activity. The manipulations were made on several positions of the paromamine core.

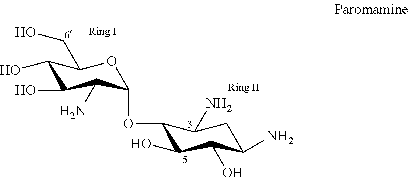

Exemplary such manipulations of the paromamine core which have been taught in these publications include a hydroxyl group at position 6′ of the aminoglycoside core; introduction of one or more monosaccharide moieties or an oligosaccharide moiety at position 3′, 4′, 3, 4, 5 and/or 6 of the aminoglycoside core; introduction of an (S)-4-amino-2-hydroxybutyryl (AHB) moiety at position N1 of the paromamine core; substitution of hydrogen at position 6′ by an alkyl such as a methyl substituent; and an introductions of an alkyl group at the 5″ position, in case a monosaccharide moiety is introduced to the paromamine core.

Studies have showed that that 2-week ((D. Wang et al., Molecular Genetics and Metabolism, 105, 116-125 (2012); K. M. Keeling, et al., PLoS ONE 8 (4), e60478 (2013)) and 28-week (G. Gunn et al., Molec. Genet. Metabol. 111, 374-381 (2014)) treatment with an exemplary compound disclosed in WO 2007/113841, NB84 (as depicted below) restored enough α-L-iduronidase function via PTC suppression to reduce tissue GAG accumulation in the Idua^{tm1Kmke} mucopolysaccharidosis type I-H (MPS I-H) mouse model, which carries a PTC homologous to the human IDUA-W402X nonsense mutation. It has also been shown that, following 28-week NB84 treatment revealed significant moderation of the disease in multiple tissues, including the brain, heart and bone, that are resistant to current MPS I-H therapies. These data demonstrate that long-term nonsense suppression therapy using aminoglycosides featuring a modified paromamine core can moderate progression of a genetic disease.

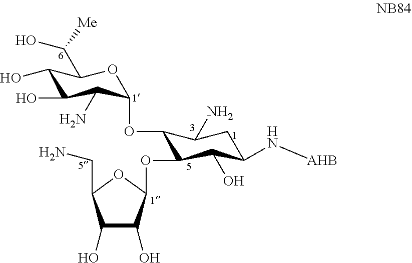

WO 2017/037717 and WO 2017/037718 disclose additional classes of paromomycin-derived aminoglycosides, designed to exhibit high premature stop codon mutations readthrough activity while exerting low cytotoxicity in mammalian cells and low antimicrobial activity, by introducing additional manipulations to the paromamine core, which lead to enhanced readthrough activity and reduced toxicity and antimicrobial activity. Exemplary such manipulations of the paromamine core which have been taught in these publications, and which can be in addition to, or instead of, the manipulations taught in WO 2007/113841 and WO 2012/066546, include further substitution of the hydroxyl group at position 6′ of the aminoglycoside core; introduction of various groups (e.g., alkyl, aryl alkaryl, acyl or cell-permealizing groups such as guanidinyl) at position N1 of the paromamine core; and an introduction of a cell-permealizable group at the 5″ position (in case a monosaccharide is attached to the paromamine core).

WO 2017/118968 discloses additional classes of paromomycin-derived aminoglycosides, designed to exhibit high premature stop codon mutations readthrough activity while exerting low cytotoxicity in mammalian cells and low antimicrobial activity, by introducing additional manipulations to the paromamine core which lead to enhanced readthrough activity and reduced toxicity and antimicrobial activity. Exemplary such manipulations of the paromamine core which have been taught in these publications, and which can be in addition to, or instead of, the manipulations taught in WO 2007/113841, WO 2012/066546, 2017/037717 and WO 2017/037718, include introduction of a hydroxyalkyl group at the 6′ position; replacing Ring I with an unsaturated ring that features a double bond between the 4′ and 5′ positions; and introducing acyl groups at various positions.

WO 2017/037719 further discloses additional classes of paromomycin-derived aminoglycosides, designed to exhibit high premature stop codon mutations readthrough activity while exerting low cytotoxicity in mammalian cells and low antimicrobial activity, by introducing additional manipulations to the paromamine core, which lead to enhanced readthrough activity and reduced toxicity and antimicrobial activity.

Huth et al. (in J. Clin. Invest. 125, 583-92 (2015)) have hypothesized that the mechanotransducer (MET) channels that are present on hair cells, and act as cationic channels, are directly involved in the entry of aminoglycosides in the cochlea (see, Background Art FIG. 1), and have suggested that preventing/inhibiting the entry of AGs through those channels can significantly reduce the ototoxic side effect of the drug. To test this hypothesis, the total positive charge of the AG sisomicin was reduced by acylation of one or two amino groups simultaneously, namely, acylation of N1 of ring II, of N3 of ring III, and both N1 and N3 simultaneously, of sisomicin, was effected and the 9 different compounds were evaluated for the antibacterial activity and the inhibition of the MET channels. It was found that the N1-methylsufonyl modified sisomicin exhibited significantly reduced ototoxicity while it also kept its antibacterial activity similar to that of the parent antibiotic sisomicin.

Additional background art includes Sabbavarapu et al., Med. Chem. Commun., 2018, 9, 503; Nudelman, I., et al., Bioorg Med Chem Lett, 2006. 16(24): p. 6310-5; Hobbie, S.N., et al., Nucleic Acids Res, 2007. 35(18): p. 6086-93; Kondo, J., et al., Chembiochem, 2007. 8(14): p. 1700-9; Rebibo-Sabbah, A., et al., Hum Genet, 2007. 122(3-4): p. 373-81; Azimov, R., et al., Am J Physiol Renal Physiol, 2008. 295(3): p. F633-41; Hainrichson, M., et al., Org Biomol Chem, 2008. 6(2): p. 227-39; Hobbie, S. N., et al., Proc Natl Acad Sci USA, 2008. 105(52): p. 20888-93; Hobbie, S.N., et al., Proc Natl Acad Sci U S A, 2008. 105(9): p. 3244-9; Nudelman, I., et al., Adv. Synth. Catal., 2008. 350: p. 1682-1688; Nudelman, I., et al., J Med Chem, 2009. 52(9): p. 2836-45; Venkataraman, N., et al., PLoS Biol, 2009. 7(4): p. e95; Brendel, C., et al., J Mol Med (Berl), 2010. 89(4): p. 389-98; Goldmann, T., et al., Invest Ophthalmol Vis Sci, 2010. 51(12): p. 6671-80; Malik, V., et al., Ther Adv Neurol Disord, 2010. 3(6): p. 379-89; Nudelman, I., et al., Bioorg Med Chem, 2010. 18(11): p. 3735-46; Warchol, M.E., Curr Opin Otolaryngol Head Neck Surg, 2010. 18(5): p. 454-8; Lopez-Novoa, J. M., et al., Kidney Int, 2011. 79(1): p. 33-45; Rowe, S. M., et al., J Mol Med (Berl), 2011. 89(11): p. 1149-61; Vecsler, M., et al., PLoS One, 2011. 6(6): p. e20733; U.S. Pat. Nos. 3,897,412, 4,024,332, 4,029,882, and 3,996,205; Greenberg et al.,' Am. Chem. Soc., 1999, 121, 6527-6541; Kotra et al., antimicrobial agents and chemotherapy, 2000, p. 3249-3256; Haddad et al., J. Am. Chem. Soc., 2002, 124, 3229-3237; Kandasamy, J. et al., J. Med. Chem. 2012, 55, pp. 10630-10643; Duscha, S. et al., MBio, 2014, 5(5), p. e01827-14; Shulman, E. et al., J Biol Chem., 2014, 289(4), pp. 2318-30; Simonson et al., ChemBioChem 3, 1223-28, 2002; Shalev et al., PNAS 110, 13333-338, (2013), M. Yusopov et al., Nature 513, 517-22 (2014); Perez-Fernandez, D. et al. at. Commun. 5, 3112 (2014); Akbergenov, R. et al. Am. Soc. Microbiol. 5, 1-10 (2014); Kato, T. et al. ACS Infect. Dis. 1, 479-486 (2016); Schalev et al. Nucleic Acids Research, 43(17), 8601-8613 (2015); Sabbavarapu et al. ACS Med. Chem. Lett. 7, 418-423 (2016); Bidou et al. RNA Biology 14, 378-388 (2017); FR Patent No. 2,427,341; JP Patent No. 04046189; Keeling et al., PLoS ONE 8(4): e60478, 2013; and Alroy et al., Abstracts/Molecular genetics and metabolism 2018, 123(2):S18. The teachings of all of these documents are incorporated by reference as if fully set forth herein.

### SUMMARY OF THE INVENTION

The present invention relates to aminoglycosides, which can be beneficially used in the treatment of genetic diseases, by exhibiting high premature stop codon mutations read-through activity, low toxicity in mammalian cells and low antimicrobial activity, as well as improved bioavailability and/or cell permeability. The presently disclosed aminoglycosides are characterized by a core structure based on Rings I, II and optionally III of paromomycin.

According to an aspect of some embodiments of the present invention there are provided compounds which are collectively represented by Formula A or B, as described herein in any of the respective embodiments.

According to an aspect of some embodiments of the present invention there are provided compounds which are collectively represented by general formula I:

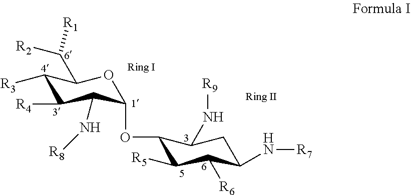

wherein:

the dashed line indicates a stereo-configuration of position 6′ being an R configuration or an S configuration;

R₁ is selected from hydrogen, a substituted or unsubstituted alkyl, a substituted or unsubstituted cycloalkyl, and a substituted or unsubstituted aryl;

R₂ is selected from hydrogen, a substituted or unsubstituted alkyl and ORx, wherein Rx is selected from hydrogen, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl, or, alternatively, R₂ is the ORx and forms together with R₃ a dioxane;

R₃ is selected from hydrogen, a substituted or unsubstituted alkyl and ORy, wherein Ry is selected from hydrogen, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl, or, alternatively, R₃ is the ORy and formed together with R₂ a dioxane;

R₄—R₆ are each independently selected from hydrogen, a substituted or unsubstituted alkyl, and ORz, wherein Rz is selected from hydrogen, a monosaccharide moiety, an oligosaccharide moiety, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl; and

R₇—R₉ are each independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a sulfonyl,

provided that at least one of R₇—R₉ is a sulfonyl.

According to some of any of the embodiments described herein, R₇ is the sulfonyl, and the compounds are collectively represented by Formula Ia:

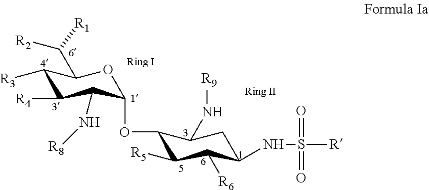

wherein:

R₁—R₆, R₈ and R₉ are as defined for Formula I; and R′ is selected from hydrogen, a substituted or unsubstituted alkyl, a substituted or unsubstituted cycloalkyl, a substituted or unsubstituted alkaryl, and a substituted or unsubstituted aryl.

According to some of any of the embodiments described herein, R′ is selected from an unsubstituted alkyl and an unsubstituted aryl.

According to some of any of the embodiments described herein, R′ is methyl.

According to some of any of the embodiments described herein, R₈ and R₉ are each hydrogen.

According to some of any of the embodiments described herein, R₂ is ORx, and Rx is selected from hydrogen and a substituted or unsubstituted alkyl.

According to some of any of the embodiments described herein, R₃ is ORy, and Ry is selected from hydrogen and a substituted or unsubstituted alkyl.

According to some of any of the embodiments described herein, R₂ and R₃ form together a dioxane.

According to some of any of the embodiments described herein, there are provided compounds which are collectively represented by Formula Ic or Id, as described herein in any of the respective embodiments.

According to an aspect of some embodiments of the present invention there are provided compounds which are collectively represented by general formula I*:

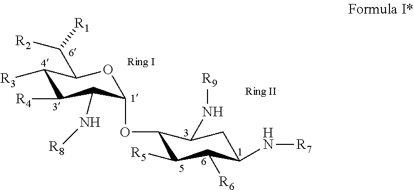

swherein:

the dashed line indicates a stereo-configuration of position 6′ being an R configuration or an S configuration;

R₁ is selected from hydrogen, a substituted or unsubstituted alkyl, a substituted or unsubstituted cycloalkyl, and a substituted or unsubstituted aryl;

R₂ is ORx, wherein Rx is selected from a substituted or unsubstituted alkyl, a substituted or unsubstituted cycloalkyl, a substituted or unsubstituted aryl, a substituted or unsubstituted heteroaryl, and a substituted or unsubstituted alkaryl;

R₃ is ORy, wherein Ry is selected from a substituted or unsubstituted alkyl, a substituted or unsubstituted cycloalkyl, a substituted or unsubstituted aryl, a substituted or unsubstituted heteroaryl, and a substituted or unsubstituted alkaryl;

R₄—R₆ are each independently selected from hydrogen, a substituted or unsubstituted alkyl, and ORz, wherein Rz is selected from hydrogen, a monosaccharide moiety, an oligosaccharide moiety, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl; and

R₇—R₉ are each independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a sulfonyl, and wherein the ORx and the ORy are linked to one another such that R₂ and R₃ form together a dioxane.

According to some of any of the embodiments described herein, the dioxane is a substituted or unsubstituted 1,3-dioxane. According to some of any of the embodiments described herein, the compounds are collectively represented by Formula I*a:

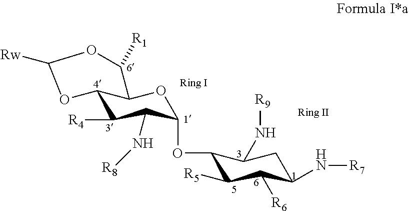

wherein:

R₁, R₄—R₆ and R₇—R₉ are as defined for Formula I*; and

Rw is selected from hydrogen, a substituted or unsubstituted alkyl, a substituted or unsubstituted cycloalkyl, a substituted or unsubstituted alkaryl, and a substituted or unsubstituted aryl.

According to some of any of the embodiments described herein, Rw is selected from a substituted or unsubstituted alkyl and a substituted or unsubstituted aryl.

According to some of any of the embodiments described herein, R₇—R₉ are each hydrogen.

According to some of any of the embodiments described herein, R₈ and R₉ are each hydrogen, and wherein R₇ is selected from hydrogen, acyl, a substituted or unsubstituted alkyl, a substituted or unsubstituted cycloalkyl, a substituted or unsubstituted alkaryl, a substituted or unsubstituted aryl, an amino-substituted alpha-hydroxy acyl and a sulfonyl.

According to some of any of the embodiments described herein, R₇ is acyl.

According to some of any of the embodiments described herein, R₇ is the sulfonyl, and the compounds are collectively represented by Formula I*b:

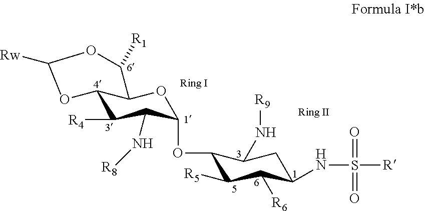

wherein:

Rw, R₁, R₄—R₆, R₈ and R₉ are as defined for Formula I*; and

R′ is selected from hydrogen, a substituted or unsubstituted alkyl, a substituted or unsubstituted cycloalkyl, a substituted or unsubstituted alkaryl, and a substituted or unsubstituted aryl.

According to some of any of the embodiments described herein, each of R₄—R₆ is ORz.

According to some of any of the embodiments described herein, each of R₄—R₆ is ORz, and in each of the R₄—R₆ Rz is hydrogen.

According to some of any of the embodiments described herein, at least one of R₄—R₆ is ORz and Rz is the monosaccharide moiety or the oligosaccharide moiety.

According to some of any of the embodiments described herein, R₅ is ORz and Rz is the monosaccharide moiety.

According to some of any of the embodiments described herein, the monosaccharide moiety is represented by Formula II:

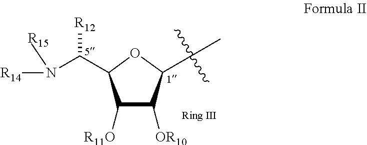

wherein the curved line denotes a position of attachment;

the dashed line indicates a stereo-configuration of position 5″ being an R configuration or an S configuration;

R₁₀ and R₁₁ are each independently selected from hydrogen, a substituted or unsubstituted alkyl, a substituted or unsubstituted cycloalkyl, a substituted or unsubstituted aryl, a substituted or unsubstituted alkaryl, and an acyl;

R₁₂ is selected from the group consisting of hydrogen, a substituted or unsubstituted alkyl, a substituted or unsubstituted cycloalkyl, and a substituted or unsubstituted aryl; and

each of R₁₄ and R₁₅ is independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl, a sulfonyl and a cell-permealizable group, or, alternatively, R₁₄ and R₁₅ form together a heterocyclic ring.

According to some of any of the embodiments described herein, R₅ is ORz and Rz is the monosaccharide moiety represented by Formula II, and the compounds are collectively represented by Formula III:

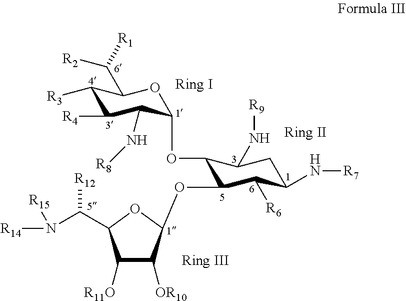

wherein:

R₁—R₄ and R₆—R₉ are each as defined for Formula I or Formula Ia; and

R₁₀, R₁₁, R₁₂, R₁₄ and R₁₅ are each as defined for Formula II.

According to some of any of the embodiments described herein, R₇ is the sulfonyl, and the compounds are collectively represented by Formula IIIa:

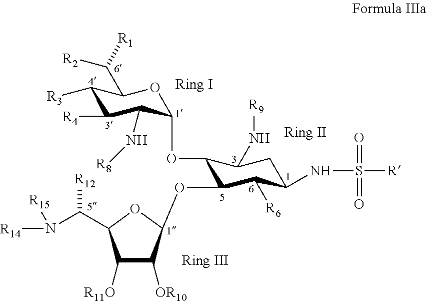

wherein:

R₁—R₄, R₆, R₈ and R₉ are as defined for Formula I or Formula Ia;

R₁₀, R₁₁, R₁₂, R₁₄ and R₁₅ are as defined for Formula II; and

R′ is selected from hydrogen, a substituted or unsubstituted alkyl, a substituted or unsubstituted cycloalkyl, a substituted or unsubstituted alkaryl, and a substituted or unsubstituted aryl.

According to some of any of the embodiments described herein, R₂ is ORx, and Rx is selected from hydrogen and a substituted or unsubstituted alkyl.

According to some of any of the embodiments described herein, R₃ is ORy, and Ry is selected from hydrogen and a substituted or unsubstituted alkyl.

According to some of any of the embodiments described herein, R₂ and R₃ form together a dioxane.

According to some of any of the embodiments described herein, R₄ and R₆ are each independently ORz.

According to some of any of the embodiments described herein, R₄ and R₆ are each ORz and Rz is hydrogen.

According to some of any of the embodiments described herein, R₈ and R₉ are each hydrogen.

According to some of any of the embodiments described herein, R₁₀, R₁₁, R₁₂, R₁₄ and R₁₅ are each hydrogen.

According to some of any of the embodiments described herein, R₁₀, R₁₁, R₁₄ and R₁₅ are each hydrogen and R₁₂ is selected from the group consisting of a substituted or unsubstituted alkyl, a substituted or unsubstituted cycloalkyl, and a substituted or unsubstituted aryl.

According to some of any of the embodiments described herein, R₁₂ is a substituted or unsubstituted alkyl.

According to some of any of the embodiments described herein, R₁₂ is methyl.

According to some of any of the embodiments described herein, the compound is selected from: NB74-MeS; NB74-PhS; NB124-MeS; and NB124-PhS, as shown hereinbelow.

According to some of any of the embodiments described herein, R₅ is ORz and Rz is the monosaccharide moiety represented by Formula II, and the compounds are collectively represented by Formula III*:

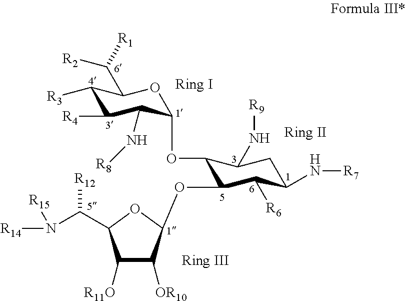

wherein:

R₁—R₄ and R₆—R₉ are each as defined for Formula I* or I*a or I*b; and

R₁₀, R₁₁, R₁₂, R₁₄ and R₁₅ are each as defined for Formula II.

According to some of any of the embodiments described herein, the dioxane is a substituted or unsubstituted 1,3-dioxane, and the compounds are collectively represented by Formula III*a:

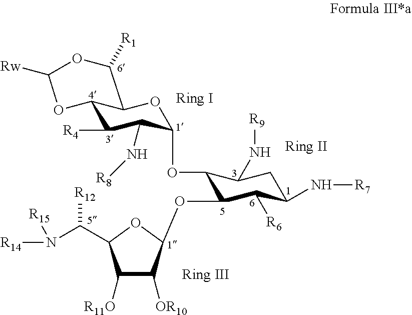

wherein:

R₁, R₄, R₆, and R₇—R₉ are as defined for Formula I* or I*a or I*b;

R₁₀, R₁₁, R₁₂, R₁₄ and R₁₅ are as defined for Formula II; and

Rw is selected from hydrogen, a substituted or unsubstituted alkyl, a substituted or unsubstituted cycloalkyl, a substituted or unsubstituted alkaryl, and a substituted or unsubstituted aryl.

According to some of any of the embodiments described herein, Rw is selected from a substituted or unsubstituted alkyl and a substituted or unsubstituted aryl.

According to some of any of the embodiments described herein, R₇—R₉ are each hydrogen.

According to some of any of the embodiments described herein, R₈ and R₉ are each hydrogen, and wherein R₇ is selected from hydrogen, acyl, a substituted or unsubstituted alkyl, a substituted or unsubstituted cycloalkyl, a substituted or unsubstituted alkaryl, a substituted or unsubstituted aryl, an amino-substituted alpha-hydroxy acyl and a sulfonyl.

According to some of any of the embodiments described herein, R₇ is acyl.

According to some of any of the embodiments described herein, R₇ is the sulfonyl, and the compounds are collectively represented by Formula III*b:

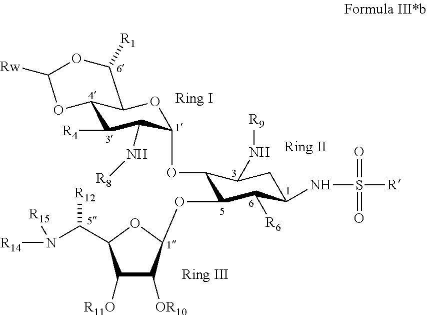

wherein:

Rw, R₁, R₄, R₆, R₈ and R₉ are as defined for Formula I*b;

R₁₀, R₁₁, R₁₂, R₁₄ and R₁₅ are as defined for Formula II; and

R′ is selected from hydrogen, a substituted or unsubstituted alkyl, a substituted or unsubstituted cycloalkyl, a substituted or unsubstituted alkaryl, and a substituted or unsubstituted aryl.

According to some of any of the embodiments described herein, R₄ and R₆ are each independently ORz.

According to some of any of the embodiments described herein, R₄ and R₆ are each ORz and Rz is hydrogen.

According to some of any of the embodiments described herein, R₁₀, R₁₁, R₁₂, R₁₄ and R₁₅ are each hydrogen.

According to some of any of the embodiments described herein, R₁₀, R₁₁, R₁₄ and R₁₅ are each hydrogen and R₁₂ is selected from the group consisting of a substituted or unsubstituted alkyl, a substituted or unsubstituted cycloalkyl, and a substituted or unsubstituted aryl.

According to some of any of the embodiments described herein, R₁₂ is a substituted or unsubstituted alkyl, e.g., methyl.

According to some of any of the embodiments described herein, R₁ is a substituted or unsubstituted alkyl.

According to some of any of the embodiments described herein, R₁ is methyl.

According to an aspect of some embodiments of the present invention there are provided compounds collectively represented by Formula IV:

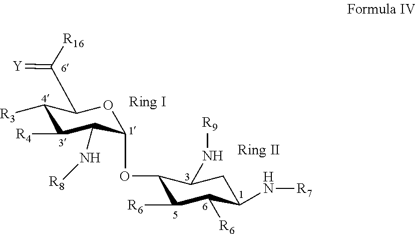

wherein:

Y is selected from oxygen and sulfur;

R₁₆ is selected from hydrogen, amine and ORq;

Rq is selected from hydrogen, a monosaccharide moiety, an oligosaccharide moiety, a substituted or unsubstituted alkyl, a substituted or unsubstituted cycloalkyl, a substituted or unsubstituted aryl, a substituted or unsubstituted heteroaryl, and a substituted or unsubstituted alkaryl;

R₃—R₆ are each independently selected from hydrogen, a substituted or unsubstituted alkyl, and ORz, wherein Rz is selected from hydrogen, a monosaccharide moiety, an oligosaccharide moiety, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl; and

R₇—R₉ are each independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a sulfonyl.

According to some of any of the embodiments described herein, Y is oxygen.

According to some of any of the embodiments described herein, R₁₆ is amine.

According to some of any of the embodiments described herein, R₁₆ is ORq and Rq is hydrogen.

According to some of any of the embodiments described herein, R₃—R₆ are each independently the ORz.

According to some of any of the embodiments described herein, in each of R₃—R₆, Rz is hydrogen.

According to some of any of the embodiments described herein, R₇—R₉ are each hydrogen.

According to some of any of the embodiments described herein, the compound is selected from NB160 and NB161, as shown in FIG. 10.

According to some of any of the embodiments described herein, at least one of R₃—R₆ is ORz, wherein Rz is a monosaccharide moiety or an oligosaccharide moiety.

According to some of any of the embodiments described herein, the Rz is a monosaccharide moiety represented by Formula II as defined in any of the respective embodiments.

According to some of any of the embodiments described herein, R₅ is ORz, and Rz is the monosaccharide moiety represented by Formula II, the compounds being collectively represented by Formula IVa:

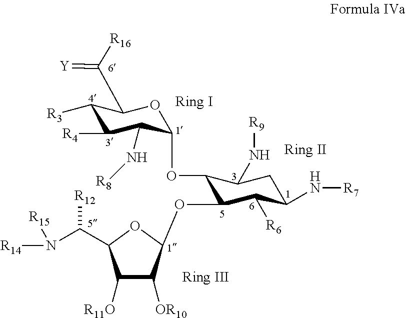

wherein:

the dashed line indicates a stereo-configuration of position 5″ being each independently an R configuration or an S configuration;

Y, R₃, R₄ and R₆—R₉ are each as defined for Formula IV;

R₁₀ and R₁₁ are each independently selected from hydrogen, a substituted or unsubstituted alkyl, a substituted or unsubstituted cycloalkyl, a substituted or unsubstituted aryl, a substituted or unsubstituted alkaryl, and an acyl;

R₁₂ is selected from the group consisting of hydrogen, a substituted or unsubstituted alkyl, a substituted or unsubstituted cycloalkyl, and a substituted or unsubstituted aryl; and

each of R₁₄ and R₁₅ is independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl, a sulfonyl and a cell-permealizable group, or, alternatively, R₁₄ and R₁₅ form together a heterocyclic ring.

According to some of any of the embodiments described herein, R₁₀, R₁₁, R₁₂, R₁₄ and R₁₅ are each hydrogen.

According to some of any of the embodiments described herein, R₁₀, R₁₁, R₁₄ and R₁₅ are each hydrogen, and wherein R₁₂ is an alkyl.

According to some of any of the embodiments described herein, the compound is selected from NB162, NB163, NB164 and NB165, as shown in FIG. 10.

According to some embodiments of the present invention there are provided processes of preparing any of the compounds as described herein, effected according to the general description and exemplary procedures as set forth hereinbelow.

According to an aspect of some embodiments of the present invention there is provided a pharmaceutical composition comprising a compound as described herein in any one of the embodiments and any combination thereof (e.g., a compound of Formula A, B, I, I*, III, III*, IV or IVa, preferably of Formula A, B, I, I*, III or III*, including any of the respective embodiments of the compounds and any combinations thereof and including compounds represented by Formula Ia, I**, I*a, I*b, IIIa, III**, III*a and, III*b), and a pharmaceutically acceptable carrier.

According to some of any of the embodiments described herein, the pharmaceutical composition is for use in the treatment of a genetic disorder associated with a premature stop-codon truncation mutation and/or a protein truncation phenotype.

According to some of any of the embodiments described herein, the pharmaceutical composition is packaged in a packaging material and identified in print, in or on the packaging material, for use in the treatment of a genetic disorder associated with a premature stop-codon truncation mutation and/or a protein truncation phenotype.

According to an aspect of some embodiments of the present invention there is provided a method for treating a genetic disorder associated with a premature stop-codon truncation mutation and/or a protein truncation phenotype, the method comprising administering to a subject in need thereof a therapeutically effective amount of a compound as described herein in any one of the embodiments and any combination thereof (e.g., a compound of Formula A, B, I, I*, III, III*, IV or IVa, preferably of Formula A, B, I, I*, III or III*, including any of the respective embodiments of the compounds and any combinations thereof and including compounds represented by Formula Ia, I**, I*a, I*b, IIIa, III**, III*a and, III*b).

According to an aspect of some embodiments of the present invention there is provided a compound as described herein in any one of the embodiments and any combination thereof (e.g., a compound of Formula A, B, I, I*, III, III*, IV or IVa, preferably of Formula A, B, I, I*, III or III*, including any of the respective embodiments of the compounds and any combinations thereof and including compounds represented by Formula Ia, I**, I*a, I*b, IIIa, III**, III*a and, III*b), for use in the treatment of a genetic disorder associated with a premature stop-codon truncation mutation and/or a protein truncation phenotype.

According to an aspect of some embodiments of the present invention there is provided a use of the compound as described herein in any one of the embodiments and any combination thereof (e.g., a compound of Formula A, B, I, I*, III, III*, IV or IVa, preferably of Formula A, B, I, I*, III or III*, including any of the respective embodiments of the compounds and any combinations thereof and including compounds represented by Formula Ia, I**, I*a, I*b, IIIa, III**, III*a and, III*b), in the manufacture of a medicament for treating a genetic disorder associated with a premature stop-codon truncation mutation and/or a protein truncation phenotype.

According to some of any of the embodiments described herein, the genetic disorder is selected from the group consisting of cystic fibrosis (CF), Duchenne muscular dystrophy (DMD), ataxia-telangiectasia, Hurler syndrome, hemophilia A, hemophilia B, Usher syndrome, Tay-Sachs, Becker muscular dystrophy (BMD), Congenital muscular dystrophy (CMD), Factor VII deficiency, Familial atrial fibrillation, Hailey—Hailey disease, McArdle disease, Mucopolysaccharidosis, Nephropathic cystinosis, Polycystic kidney disease, Rett syndrome, Spinal muscular atrophy (SMA), cystinosis, Severe epidermolysis bullosa, Dravet syndrome, X-linked nephrogenic diabetes insipidus (XNDI), X-linked retinitis pigmentosa and cancer.

According to an aspect of some embodiments of the present invention there is provided a method of increasing the expression level of a gene having a premature stop-codon mutation, the method comprising translating the gene into a protein in the presence of a compound as described herein in any of the respective embodiments and any combination thereof (e.g., a compound of Formula A, B, I, I*, III, III*, IV or IVa, preferably of Formula A, B, I, I*, III or III*, including any of the respective embodiments of the compounds and any combinations thereof and including compounds represented by Formula Ia, I**, I*a, I*b, IIIa, III**, III*a and, III*b).

According to an aspect of some embodiments of the present invention there is provided a compound as described herein in any of the respective embodiments and any combination thereof (e.g., a compound of Formula A, B, I, I*, III, III*, IV or IVa, preferably of Formula A, B, I, I*, III or III*, including any of the respective embodiments of the compounds and any combinations thereof and including compounds represented by Formula Ia, I**, I*a, I*b, IIIa, III**, III*a and, III*b) for use in increasing the expression level of a gene having a premature stop-codon mutation.

According to an aspect of some embodiments of the present invention there is provided a use of a compound as described herein in any of the respective embodiments and any combination thereof (e.g., a compound of Formula A, B, I, I*, III, III*, IV or IVa, preferably of Formula A, B, I, I*, III or III*, including any of the respective embodiments of the compounds and any combinations thereof and including compounds represented by Formula Ia, I**, I*a, I*b, IIIa, III**, III*a and, III*b) in the manufacture of a medicament for increasing the expression level of a gene having a premature stop-codon mutation.

According to some of any of the embodiments described herein, the premature stop-codon mutation has an RNA code selected from the group consisting of UGA, UAG and UAA.

According to some of any of the embodiments described herein, the protein is translated in a cytoplasmic translation system.

According to some of any of the embodiments described herein, the compound is used in a mutation suppression amount.

According to some of any of the embodiments described herein, an inhibition of translation IC₅₀ of the compound in a eukaryotic cytoplasmic translation system is greater that an inhibition of translation IC₅₀ of the compound in a ribosomal translation system.

According to some of any of the embodiments described herein, an inhibition of translation IC₅₀ of the compound in a eukaryotic cytoplasmic translation system is greater that an inhibition of translation IC₅₀ of the compound in a prokaryotic translation system. According to an aspect of some embodiments of the present invention there is provided a compound as described herein in any of the respective embodiments and any combination thereof (e.g., a compound of Formula A, B, I, I*, III, III*, IV or IVa, preferably of Formula A, B, I, I*, III or III*, including any of the respective embodiments of the compounds and any combinations thereof and including compounds represented by Formula Ia, I**, I*a, I*b, IIIa, III**, III*a and, III*b) for use in attenuating nonsense mutation mRNA decay (NMD) and/or in treating a disease or disorder in which attenuating NMD is beneficial (e.g., cancer).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 (Background Art) presents the structure of the aminoglycoside Sisomicin structure (left) and a schematic illustration showing that MET channels act as cationic channel (A, right), as reported in Huth et al., J. Clin. Invest. 125, 583-92 (2015).

FIG. 2 presents the chemical structures of exemplary compounds, featuring a substitution at the N1 position, according to some embodiments of the present invention, referred to herein also as Sett and Set2 structures.

FIG. 3 presents a scheme illustrating a general synthetic pathway for the preparation of exemplary compounds, featuring a substitution at the N1 position, according to some embodiments of the present invention and referred to herein also as Sett and Set2 structures.

FIG. 4 presents a scheme describing the synthesis of an exemplary Acceptors featuring a substitution at the N1 position.

FIG. 5 presents a scheme describing an exemplary synthesis of pseudo- trisaccharide aminoglycosides featuring a substitution at the N1 position according to exemplary embodiments of the present invention.

FIG. 6 presents a scheme describing an exemplary synthesis of pseudo-amino disaccharides featuring a substitution at the N1 position according to exemplary embodiments of the present invention

FIGS. 7A-D present the comparative plots showing the in vitro readthrough activity data of exemplary compounds, featuring a substitution at the N1 position, according to some embodiments of the present invention, as depicted in FIG. 2, compared to their respective parent compounds (lacking the N1 substitution). FIGS. 7A and 7B present the activity of NB74, NB74-MeS, NB74-PhS and NB74-Ac in readthrough of the R3X mutation (FIG. 7A) and G542X mutation (FIG. 7B). FIGS. 7C and 7D present the activity of NB124, NB124-MeS and NB124-Ac in readthrough of the R3X mutation (FIG. 7C) and G542X mutation (FIG. 7D). The mutations R3X and G542X represent nonsense mutation context constructs (UGAC and UGAG, respectively) of the genetic diseases Usher syndrome and CF, respectively.

FIG. 8 presents dose-response curves of missing outer hair cells as a function of the tested aminoglycoside; NB74-MeS (upper panel, left), NB74-Ac (upper panel, middle), NB74-PhS (upper panel, right), NB124-Ac (lower panel, left) and NB124-MeS (lower panel, right. Hair cell loss was quantified along the entire length of cochlear explants, and the concentration at 50% loss of hair cells (LC50^{Coch}) was demonstrated by Grafit5 software.

FIGS. 9A-D present hair cell loss in cochlear explants in the present of exemplary aminoglycoside compounds according to some of the present embodiments. Explants of the mouse organ of Corti were incubated with drugs for 72 hours and stained for actin. Sections of the basal part are shown for non-treated, control explants (FIG. 9A), and for explants treated with 15 μM of NB124 (FIG. 9B), 150 μM of NB124-Ac (FIG. 9C) and 15 μM of NB124-MeS (FIG. 9D) and indicate that NB124-MeS and NB124-Ac show essentially normal morphology. Arrows in FIG. 9D point on small areas of missing outer hair cells.

FIG. 10 presents the chemical structures of exemplary compounds, featuring a carboxyl-containing (e.g., carboxylate and amide) substitutions at the 6′ position, according to some embodiments of the present invention.

FIG. 11 presents a scheme describing exemplary synthetic pathways for the preparation of exemplary acceptor compounds and of exemplary pseudo-disaccharide compounds, featuring a carboxyl-containing (e.g., carboxylate and amide) substitutions at the 6′ position, according to some embodiments of the present invention.

FIG. 12 presents a scheme describing an exemplary synthesis of pseudo- trisaccharide aminoglycosides featuring a carboxyl-containing (e.g., carboxylate and amide) substitutions at the 6′ position, according to some embodiments of the present invention.

FIG. 13 presents the chemical structures of exemplary compounds, featuring substitution(s) at the 4′ position or at the 4′ and 6′ positions, according to some embodiments of the present invention, referred to herein also as Set3 and Set4 structures.

FIG. 14 presents the chemical structures of exemplary compounds, featuring substitution(s) at the 4′ position or at the 4′ and 6′ positions, and a substitution at the N1 position, according to some embodiments of the present invention, referred to herein also as Set5 and Seth structures.

FIG. 15 presents a scheme illustrating a general synthetic pathway for the preparation of exemplary compounds, featuring substitutions at the 4′ and 6′ positions, according to some embodiments of the present invention, referred to herein also as Set3.

FIG. 16 presents schemes describing exemplary synthetic pathways for the preparation of Acceptor compounds, featuring substitutions at the 4′ and 6′ positions, according to some embodiments of the present invention; shown in the inset are structures of exemplary donor compounds according to some embodiments of the present invention.

FIG. 17 presents a scheme illustrating a general synthetic pathway for the preparation of exemplary compounds, featuring a substitution at the 4′ position, according to some embodiments of the present invention, referred to herein also as Set4.

FIGS. 18A-B present schemes describing exemplary synthetic pathways for the preparation of acceptor compounds, featuring a substitution at the 4′ position, according to some embodiments of the present invention.

FIG. 19 presents a scheme illustrating general synthetic pathways for the preparation of exemplary compounds, featuring substitution(s) at the 4′ position or at the 4′ and 6′ positions, and a substitution at the N1 position, according to some embodiments of the present invention, referred to herein also as Set5 and Seth structures.

FIG. 20 presents a scheme describing an exemplary synthesis of an acceptor compound, featuring substitutions at the 4′ and 6′ positions, and a substitution at the N1 position, according to some embodiments of the present invention.

### DESCRIPTION OF SPECIFIC EMBODIMENTS OF THE INVENTION

The present invention, in some embodiments thereof, relates to aminoglycosides and more particularly, but not exclusively, to novel aminoglycoside derivatives and their use in increasing an expression of a gene having a stop codon mutation and/or in the treatment of genetic disorders.

Specifically, the present invention, in some embodiments thereof, relates to novel aminoglycoside compounds, derived from paromomycin, which exhibit high premature stop codon mutation readthrough activity, and which are characterized by reduced toxicity, e.g., ototoxicity, in mammalian cells. Embodiments of the present invention are further of pharmaceutical compositions containing these compounds, and of uses thereof in the treatment of genetic disorders. Embodiments of the present invention are further of processes of preparing these compounds.

The principles and operation of the present invention may be better understood with reference to the figures and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As discussed hereinabove, the use aminoglycosides as therapeutic agents is limited primarily due to their high toxicity. In the context of treatment of genetic disorders, such a use is further limited by the antibacterial activity exhibited by the aminoglycosides, which can also translate into toxicity.

Additional limitations associated with aminoglycosides include low bioavailability, which typically requires an intravenous or subcutaneous administration, and poor permeability into eukaryotic cells, which typically requires administration of high doses which are associated with adverse side effected. It is assumed that the high water solubility and polarity of aminoglycosides limits their absorbance through intestinal tissues and their permeability through cell membranes.

As further discussed hereinabove, several structural manipulations on the structure of paromamine have given rise to synthetic aminoglycosides which have been shown to exert improved premature stop codon mutations readthrough activity while exerting low toxicity in mammalian cells. WO 2007/113841, WO 2012/066546, WO 2017/037717, WO 2017/037718, WO 2017/037719 and WO 2017/118968, which are incorporated by reference as if fully set forth herein, describe such aminoglycosides. Two of the promising drug candidates disclosed in these documents are NB74 and NB124, the structures of which are presented hereinbelow.

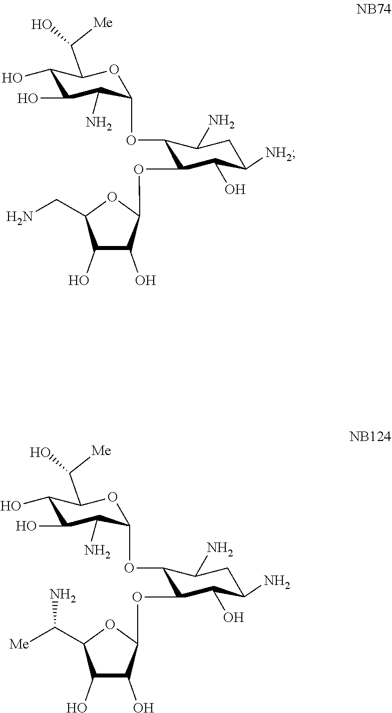

While further deciphering the structure-activity relationship of such aminoglycosides, in an attempt to further improve their therapeutic effect in the context of genetic disorders, the present inventors have designed numerous additional modifications, at varying positions of the paromamine structure, which are collectively represented herein by Formulae A, B, I, I*, III, III*, IV and IVa. The present inventors have studied the effect of these modifications on the readthrough activity and toxicity of the designed compounds, particularly when compared to the previously disclosed modified aminoglycosides featuring a paromamine core (e.g., NB74 and NB124), with the aim of uncovering compounds that feature an improved therapeutic index, that is, which exhibit a readthrough activity at least as high as that of the previously disclosed modified aminoglycosides, yet exhibit a reduced toxicity (e.g., ototoxicity).

While reducing the present invention to practice, exemplary novel aminoglycoside structures were designed and successfully practiced. As demonstrated in the Examples section that follows, these compounds were shown to exhibit high readthrough activity of disease-causing nonsense mutations as well as reduced toxicity.

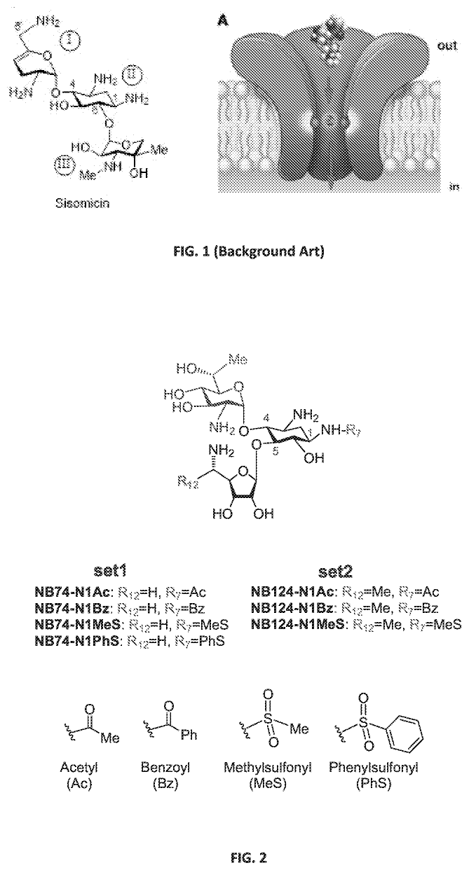

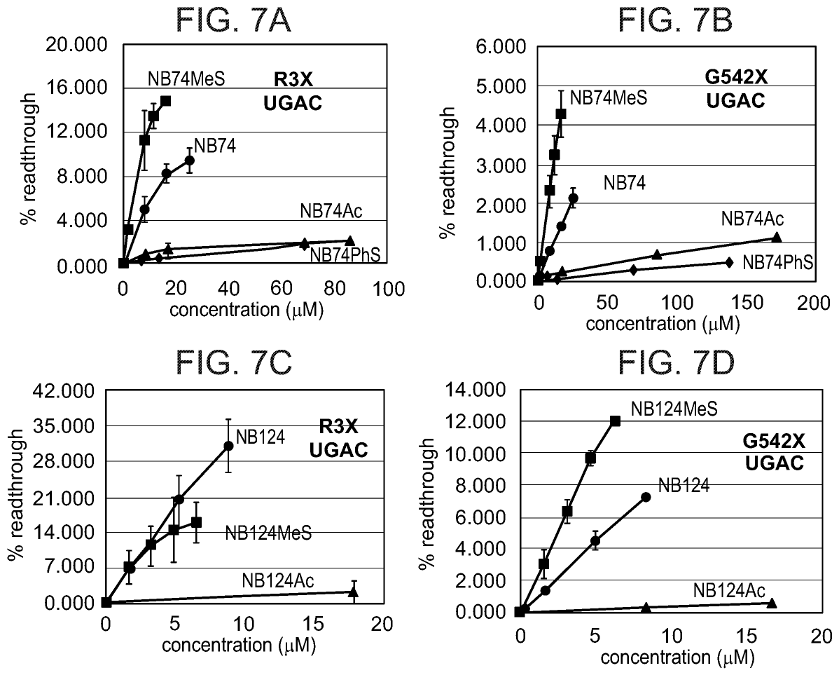

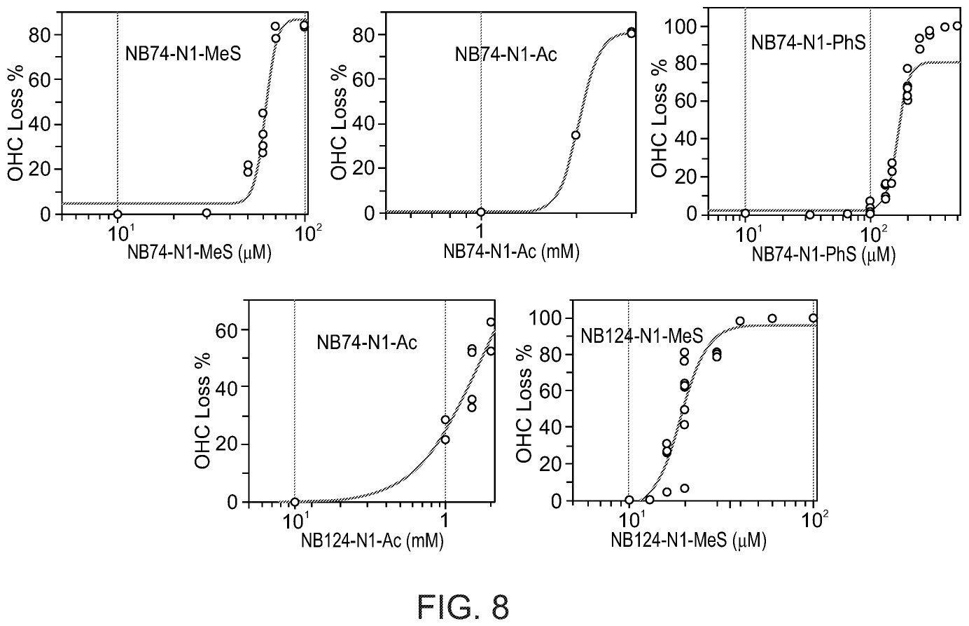

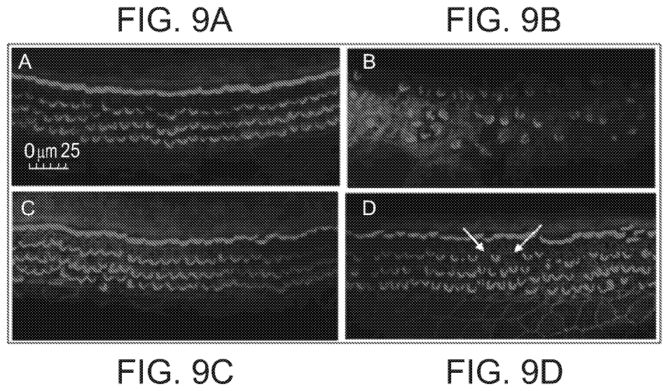

More specifically, it has been demonstrated that exemplary compounds featuring a sulfonyl substitution at the N1 position of pseudo di- and tri-saccharides featuring a paromamine core (optionally in addition to previously described modifications of the paromamine core), as shown in FIG. 2, at least maintain the high readthrough activity of previously disclosed aminoglycosides (e.g., NB74 and NB124, supra), as shown in FIGS. 7A-D, yet exhibit as substantially reduced ototoxicity, as shown in FIGS. 8 and 9A-D.

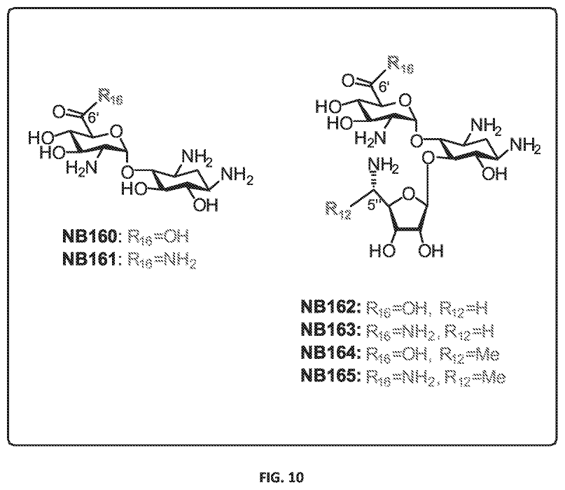

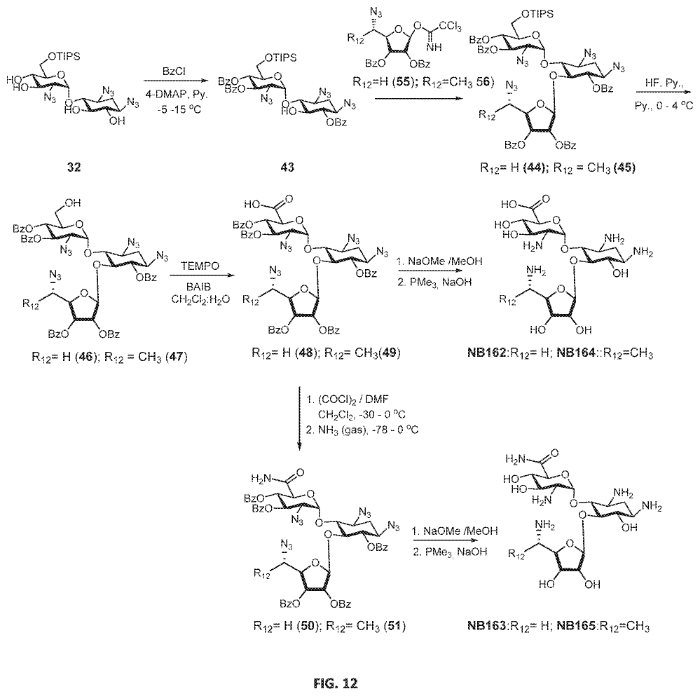

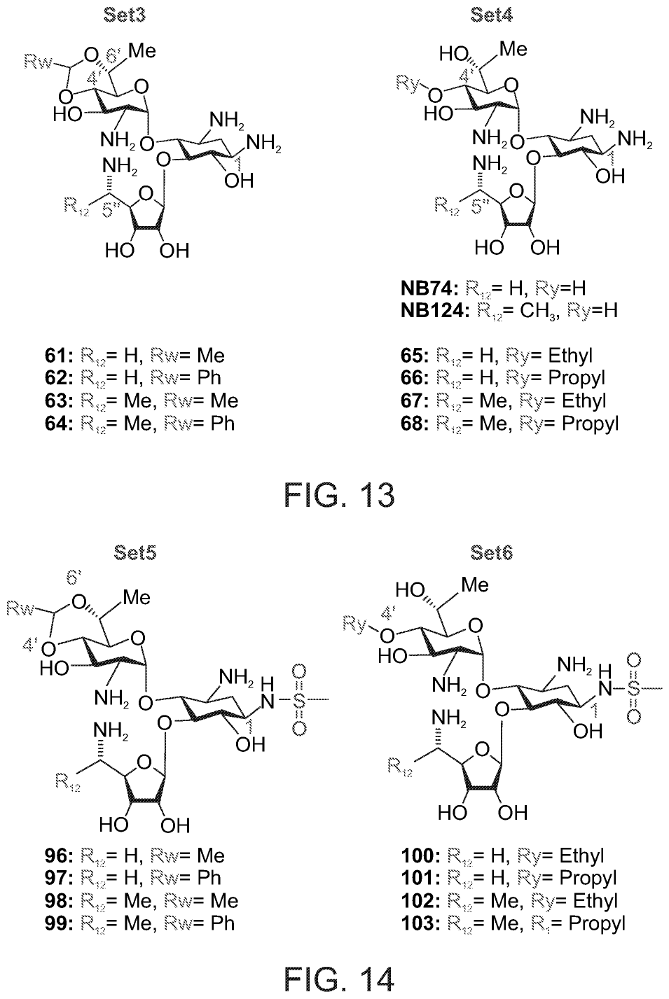

The design and practice of exemplary compounds featuring modifications at the 4′ and/or 6′ positions have also been demonstrated, as shown in FIGS. 10 and 13, optionally in combination with a sulfonyl substitution at the N1 position, as shown in FIG. 14.

Embodiments of the present invention therefore relate to novel aminoglycoside (AMG) compounds (also referred to herein as “aminoglycoside derivatives” or “modified aminoglycosides”), which are collectively represented by Formulae A, B, I, I*, III, III*, IV or IVa, to processes of preparing same and to the use thereof as inducers of readthrough of premature stop codon and/or protein truncation mutations and hence in the treatment of genetic diseases and disorders associated with such mutations.

The Compounds:

The novel aminoglycoside derivatives of the present embodiments feature a paromamine core, as previously described, while introducing thereto modifications at least at one or more of the C4′, C6′ and N1 positions, optionally in combination with additional modifications, such as the previously described modifications at C6′, C5, N1 and 5″, in case the aminoglycoside is a pseudo trisaccharide.

According to some embodiments of the present invention, the AMG derivatives as described herein feature a modification at the N1 position, and according to some of these embodiments, the amine at the N1 position is substituted by an acyl or by a sulfonyl. Such AMG compounds are also referred to herein as N1-substituted compounds. Exemplary such compounds are presented herein as Sett and Set2 compounds (see, for example, FIG. 2).

According to some embodiments of the present invention, the AMG derivatives as described herein feature a modification at one or more of the N1 position, the N2′ position, the N3 position and, optionally, in case of a pseudo-trisaccharide, at the N5″ position, and according to some of these embodiments, one or more of the amines at these positions is substituted by an acyl or by a sulfonyl. Such AMG compounds are also referred to herein as amine-substituted compounds.

According to some embodiments of the present invention, the AMG derivatives as described herein feature a modification at the C4′ position, and according to some of these embodiments, the AMG compounds feature an alkoxy or an aryloxy at the C4′ position. Such AMG compounds are also referred to herein as C4′-modified compounds. Exemplary such compounds are presented herein as Set4 and Seth compounds (see, for example, FIGS. 13 and 14, respectively).

According to some embodiments of the present invention, the AMG derivatives as described herein feature a modification at the C4′ and C6′ positions, and according to some of these embodiments, the C4′ and C6′ form a part of a dioxane ring, as defined herein. Such AMG compounds are also referred to herein as C4′, C6′-modified compounds. Exemplary such compounds are presented herein as Set3 and Set5 compounds (see, for example, FIGS. 13 and 14, respectively).

According to some embodiments of the present invention, the AMG derivatives as described herein feature a modification at the C6′ position, and according to some of these embodiments, the AMG feature a carboxyl-containing group (e.g., a carboxylate or amide, as defined herein) at the C6′ position. Such AMG compounds are also referred to herein as C6′-modified compounds.

According to some embodiments of the present invention, the AMG derivatives as described herein feature a modification at the C4′ position, the C4′ and C6′ positions or the C6′ position, as described herein, in combination with a modification at one or more of the N1 position, the N2′ position, the N3 position and, optionally, in case of a pseudo-trisaccharide, at the N5″ position. Exemplary such compounds are presented herein as Set5 and Seth compounds (see, for example, FIG. 14).

According to some of any of the embodiments described herein, the AMG derivatives as described herein further feature a modification of the paromamine core at the C6′ position, when applicable, by introducing at this position an alkyl, cycloalkyl or aryl substituent, as previously described.

According to some of any of the embodiments described herein, the AMG derivatives as described herein are pseudo-disaccharides.

According to some of any of the embodiments described herein, the AMG derivatives as described herein are pseudo-trisaccharides and thereby feature a further modification of the paromamine core by introducing thereto a monosaccharide moiety. In some of these embodiments, the AMG further feature a modification at the 5″ position, by introducing at this position an alkyl, cycloalkyl or aryl substituent, as previously described.

According to an aspect of some embodiments of the present invention, there are provided AMG compounds which are collectively represented by Formula A:

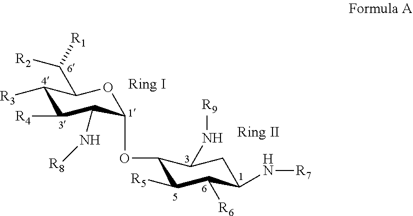

wherein:

the dashed line indicates a stereo-configuration of position 6′ being an R configuration and an S configuration (in case R₁ and R₂ are each other than hydrogen);

R₁ is selected from hydrogen, a substituted or unsubstituted alkyl, a substituted or unsubstituted cycloalkyl, and a substituted or unsubstituted aryl;

R₂ is selected from hydrogen, a substituted or unsubstituted alkyl and ORx, wherein Rx is selected from hydrogen, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl, or, alternatively, R₂ and R₃ form together a dioxane ring, as described herein in any of the respective embodiments;

R₃ is selected from hydrogen, a substituted or unsubstituted alkyl and ORy, wherein Ry is selected from hydrogen, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl, or, alternatively, R₂ and R₃ form together a dioxane, as described herein in any of the respective embodiments;

R₄—R₆ are each independently selected from hydrogen, a substituted or unsubstituted alkyl, and ORz, wherein Rz is selected from hydrogen, a monosaccharide moiety, an oligosaccharide moiety, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl; and

R₇—R₉ are each independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a sulfonyl,

provided that:

(i) at least one of R₇—R₉ is a sulfonyl; and/or

(ii) at least one of R₇—R₉ is an acyl,

such that the AMG compounds feature a modification at one or more of the amines at positions N1, N3 and N2′, as described herein,

and/or provided that:

(iii) R₂ and R₃ form together the dioxane ring, as defined herein in any of the respective embodiments; or

(iv) R₃ is ORz as defined herein, and Rz is selected from a substituted or unsubstituted alkyl, a substituted or unsubstituted cycloalkyl, a substituted or unsubstituted aryl, a substituted or unsubstituted heteroaryl, and a substituted or unsubstituted alkaryl,

such that the AMG compounds feature a modification at the C4′ position or at the C4′ and C6′ positions, as described herein.

According to some of any of the embodiments described herein, the AMG compounds according to the present embodiments are collectively represented by Formula A as described herein, provided that:

(i) at least one of R₇—R₉ is a sulfonyl; and/or

(iii) R₂ and R₃ form together the dioxane ring, as defined herein in any of the respective embodiments.

According to some of any of the embodiments described herein, the AMG compounds according to the present embodiments are collectively represented by Formula A as described herein, provided that:

(i) at least one of R₇—R₉ is an acyl, and provided that:

(iii) R₂ and R₃ form together the dioxane ring, as defined herein in any of the respective embodiments; or

(iv) R₃ is ORz as defined herein, and Rz is selected from a substituted or unsubstituted alkyl, a substituted or unsubstituted cycloalkyl, a substituted or unsubstituted aryl, a substituted or unsubstituted heteroaryl, and a substituted or unsubstituted alkaryl.

According to some of any of the embodiments described herein, the AMG compounds according to the present embodiments are collectively represented by Formula A as described herein, provided that:

(i) at least one of R₇—R₉ is a sulfonyl; and/or

(ii) at least one of R₇—R₉ is an acyl,

and provided that:

(iv) R₃ is ORz as defined herein, and Rz is selected from a substituted or unsubstituted alkyl, a substituted or unsubstituted cycloalkyl, a substituted or unsubstituted aryl, a substituted or unsubstituted heteroaryl, and a substituted or unsubstituted alkaryl.

According to some of any of the embodiments described herein, the AMG compounds according to the present embodiments are collectively represented by Formula A as described herein, provided that:

(ii) at least one of R₇—R₉ is an acyl,

and provided that:

(iv) R₃ is ORz as defined herein, and Rz is selected from a substituted or unsubstituted alkyl, a substituted or unsubstituted cycloalkyl, a substituted or unsubstituted aryl, a substituted or unsubstituted heteroaryl, and a substituted or unsubstituted alkaryl.

Herein throughout, in the context of embodiments pertaining to one or more of R₇—R₉ being a sulfonyl, the term “sulfonyl” describes a —S(═O)₂—R′ group, wherein R′ in the one or more sulfonyl groups is independently hydrogen, a substituted or unsubstituted alkyl, a substituted or unsubstituted cycloalkyl, a substituted or unsubstituted aryl, a substituted or unsubstituted heteroaryl, or a substituted or unsubstituted alkaryl.

In some of any of the embodiments described herein, the sulfonyl is an alkyl sulfonyl, such that R′ is a substituted or unsubstituted alkyl. In some of these embodiments, the alkyl is an unsubstituted alkyl. In some of any of these embodiments, the alkyl is of 1 to 10, of 1 to 8, or of 1 to 6, or of 1 to 4 carbon atoms in length. In some embodiments, the alkyl is methyl.

In some of any of the embodiments described herein, the sulfonyl is an aryl sulfonyl, such that R′ is a substituted or unsubstituted aryl (e.g., phenyl). In some of these embodiments, the aryl is an unsubstituted aryl (e.g., an unsubstituted phenyl). Herein throughout, in the context of embodiments pertaining to one or more of R₇—R₉ being a sulfonyl, in cases where two or more of R₇—R₉ are each a sulfonyl, the sulfonyl groups can be the same (R′ in each sulfonyl is the same), or different (R′ in two or more of the sulfonyl groups being different).

Herein throughout, the term “dioxane”, which is also referred to herein as “dioxane ring” or “dioxane moiety”, describes a heteroalicyclic group or moiety, as defined herein, which contains at least two oxygen atoms that form a part of the ring. In the context of any of the embodiments pertaining to R₂ and R₃ forming a dioxane, the ring is preferably of at least 6 atoms, and can be a 6-membered, a 7-membered, an 8-membered, a 9-membered, a 10-membered, or higher, ring. In the context of any of the embodiments pertaining to R₂ and R₃ forming a dioxane, the dioxane is a 1,3-dioxane, in which the two oxygen atoms are separated by one carbon atom. Alternatively, the two oxygen atoms in the dioxane can be separated by 2, 3, 4, 5 or more carbon atoms.

Herein throughout, the phrase “R₂ and R₃ form together a dioxane” means that R₂ is ORx, as defined herein, R₃ is ORy, as defined herein, and Rx and Ry are linked to one another to a form a dioxane. When R₂ and R₃ form a dioxane, none of Rx and Ry is hydrogen. When R₂ and R₃ form a dioxane, the at least two oxygen atoms of the dioxane are derived from ORx and ORy. The number of carbon atoms separating the oxygen atoms in the dioxane can be determined by Rx and/or Ry. In cases where ORx and ORy are linked together to form a 1,3-dioxane, one of Rx and Ry is a methyl and the other is absent.

In some of any of the embodiments pertaining to R₂ and R₃ forming a dioxane, Rz and Ry form a hydrocarbon group or moiety, as defined herein, linking the at least two oxygen atoms deriving from ORx and ORy.

Herein, the term “hydrocarbon” or “hydrocarbon radical” describes an organic moiety that includes, as its basic skeleton, a chain of carbon atoms, also referred to herein as a backbone chain, substituted mainly by hydrogen atoms. The hydrocarbon can be saturated or non-saturated, be comprised of aliphatic, alicyclic and/or aromatic moieties, and can optionally be substituted by one or more substituents (other than hydrogen). A substituted hydrocarbon may have one or more substituents, whereby each substituent group can independently be, for example, alkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine, and any other substituents as described herein.

The hydrocarbon moiety can optionally be interrupted by one or more heteroatoms other than oxygen, including, without limitation, one or more nitrogen (substituted or unsubstituted, as defined herein for —NR′—) and/or sulfur atoms.

In some embodiments of any of the embodiments described herein relating to a hydrocarbon, the hydrocarbon is not interrupted by any heteroatom, nor does it comprise heteroatoms in its backbone chain, and can be an alkylene chain, or be comprised of alkyls, cycloalkyls, aryls, alkenes and/or alkynes, covalently attached to one another in any order.

In some of any of the embodiments described herein, R₂ and R₃ form a 1,3-dioxane, which is also referred to herein as a heterocyclic acetal.

Herein throughout, the phrase “heterocyclic acetal”, which is also referred to herein as “cyclic acetal”, describes a cyclic group in which an acetal carbon, and the two oxygen atoms linked thereto, form a part of a heteroalicyclic ring, or, in other words, this phrase describes a heteroalicyclic ring containing at least two oxygen atoms that are linked to one another via one carbon atom.

The phrase “1,3-dioxane” as used herein generally describes a dioxane, as defined herein, in which the at least oxygen atoms are linked to the same carbon atom. This term encompasses any dioxane ring, as described herein.

In some of any of the embodiments described herein, the dioxane is a 6-membered ring, and in some embodiments it is a 6-membered 1,3-dioxane (a heterocyclic acetal). In these embodiments, Rx and Ry form together a hydrocarbon of one carbon atom, which is substituted or unsubstituted, as defined herein.

Herein throughout, in the context of embodiments pertaining to one or more of R₇—R₉ being an acyl, the term the term “acyl” describes a —C(═O)—R′ group, wherein R′ is as described herein. In some of these embodiments, the acyl is such that R′ is a substituted or unsubstituted alkyl or a substituted or unsubstituted aryl. In some of these embodiments, R′ is an unsubstituted alkyl, preferably a short alkyl, of 1-4 carbon atoms in length. In some of these embodiments, the acyl is an unsubstituted aryl such as an unsubstituted phenyl.

In some of any of the embodiments described herein, the compound is a pseudo-disaccharide, having Ring I and Ring II as depicted in Formula A.

In these embodiments, none of R₄—R₆ is ORz in which Rz is a monosaccharide or an oligosaccharide moiety.

In some of these embodiments, one or more, or all, of R₄—R₆ is ORz, and Rz is other than a monosaccharide or an oligosaccharide.

In some of these embodiments, one or more, or all, of R₄—R₆ is ORz and Rz in each of R₄—R₆ is hydrogen. In these embodiments, one or more, or all, of R₄—R₆ is hydroxy.

Alternatively, one or more, or all, of R₄—R₆ is ORz and Rz in one or more, or all, of R₄—R₆ is other than hydrogen.

For example, in some embodiments, one or more, or all, of R₄—R₆ is ORz and Rz in one or more, or all, of R₄—R₆ is independently an alkyl, which can be substituted or unsubstituted. In these embodiments, one or more, or all, of R₄—R₆ is an alkoxy, as defined herein.

For example, in some embodiments, one or more, or all, of R₄—R₆ is ORz and Rz in one or more, or all, of R₄—R₆ is independently an aryl, which can be substituted or unsubstituted. In these embodiments, one or more, or all, of R₄—R₆ is an aryloxy, as defined herein.

In some of these embodiments, the aryl is unsubstituted such that one or more, or all of R₄—R₆, independently, can be, as non-limiting examples, phenyloxy, 1-anthryloxy, 1-naphthyloxy, 2-naphthyloxy, 2-phenanthryloxy and 9-phenanthryloxy.

In some of these embodiments, one or more of the aryls in one or more of ORz is a substituted aryl, such that one or more, or all of R₄—R₆, independently, can be, as non-limiting examples, an aryloxy in which the aryl is 2-(N-ethylamino)phenyl, 2-(N-hexylamino)phenyl, 2-(N-methylamino)phenyl, 2,4-dimethoxyphenyl, 2-acetamidophenyl, 2-aminophenyl, 2-carboxyphenyl, 2-chlorophenyl, 2-ethoxyphenyl, 2-fluorophenyl, 2-hydroxymethylphenyl, 2-hydroxyphenyl, 2-hydroxyphenyl, 2-methoxycarbonylphenyl, 2-methoxyphenyl, 2-methylphenyl, 2-N,N-dimethylaminophenyl, 2-trifluoromethylphenyl, 3-(N,N-dibutylamino)phenyl, 3-(N,N-diethylamino)phenyl, 3,4,5-trimethoxyphenyl, 3,4-dichlorophenyl, 3,4-dimethoxyphenyl, 3,5-dimethoxyphenyl, 3-aminophenyl, 3-biphenylyl, 3-carboxyphenyl, 3-chloro-4-methoxyphenyl, 3-chlorophenyl, 3-ethoxycarbonylphenyl, 3-ethoxyphenyl, 3-fluorophenyl, 3-hydroxymethylphenyl, 3-hydroxyphenyl, 3-isoamyloxyphenyl, 3-isobutoxyphenyl, 3-isopropoxyphenyl, 3-methoxyphenyl, 3-methylphenyl, 3-N,N-dimethylaminophenyl, 3-tolyl, 3-trifluoromethylphenyl, 4-(benzyloxy)phenyl, 4-(isopropoxycarbonyl)phenyl, 4-(N,N-diethylamino)phenyl, dihexylamino)phenyl, 4-(N,N-diisopropylamino)phenyl, 4-(N,N-dimethylamino)phenyl, (N,N-di-n-pentylamino)phenyl, 4-(n-hexyloxycarbonyl)phenyl, 4-(N-methylamino)phenyl, 4-(trifluoromethyl)phenyl, 4-aminophenyl, 4-benzyloxyphenyl, 4-biphenylyl, 4-butoxyphenyl, 4-butyramidophenyl, 4-carboxyphenyl, 4-chlorophenyl, 4-ethoxycarbonylphenyl, 4-hexanamidophenyl, 4-hydroxymethylphenyl, 4-hydroxyphenyl, 4-iodophenyl, 4-isobutylphenyl, 4-isobutyramidophenyl, 4-isopropoxyphenyl, 4-isopropylphenyl, 4-methoxyphenyl, 4-methylphenyl, 4-n-hexanamidophenyl, 4-n-hexyloxyphenyl, 4-n-hexylphenyl, 4-nitrophenyl, 4-nitrophenyl, 4-propionamidophenyl, 4-tolyl, 4-trifluoromethylphenyl and/or 4-valeroyloxycarbonylphenyl.

In some of these embodiments, one or more, or all, of R₄—R₆ is ORz and Rz is independently a heteroaryl, which can be substituted or unsubstituted. In these embodiments, one or more, or all, of R₄—R₆ is a heteroaryloxy, as defined herein.

In some embodiments, one or more, or all of R₄—R₆, independently, can be, as non-limiting examples, 2-anthryloxy, 2-furyloxy, 2-indolyloxy, 2-naphthyloxy, 2-pyridyloxy, 2-pyrimidyloxy, 2-pyrryloxy, 2-quinolyloxy, 2-thienyloxy, 3-furyloxy, 3-indolyloxy, 3-thienyloxy, 4-imidazolyloxy, 4-pyridyloxy, 4-pyrimidyloxy, 4-quinolyloxy, 5-methyl-2-thienyloxy and 6-chloro-3-pyridyloxy.

In some of any of the embodiments described herein, R₃ is aryloxy or heteroaryloxy, as described herein.

In some of any of the embodiments described herein, R₃ is ORy and Ry is a substituted or unsubstituted alkyl or alkenyl, for example, methyl, ethyl, propyl, butyl, pentyl, propenyl, 2-hydroxyethyl, 3-hydroxypropyl, 2,3-dihydroxypropyl and methoxymethyl.

In some of any of the embodiments described herein, R₃ is ORy and Ry is hydrogen.

In some of any of the embodiments described herein, R₄ is ORz and Rz is hydrogen.

In some of any of the embodiments described herein, R₆ is ORz and Rz is hydrogen.

In some of any of the embodiments described herein, one or more of, or all, of R₄—R₆ are ORz.

In some of any of the embodiments described herein, when one or more, or all, of R₄—R₆ is ORz and when one or more, or all, of the Rz moiety is other than hydrogen, Rz can be the same or different for each of R₄—R₆.

In some of these embodiments, when in one or more, or all, of R₄—R₆, Rz is other than hydrogen, Rz can be, for example, independently, alkyl, alkenyl, alkynyl, cycloalkyl, aryl or heteroaryl, each being optionally substituted, as described herein.

In some of any of the embodiments described herein, when one or more, or all, of R₄—R₆ is ORz, Rz is independently an acyl, as defined herein, forming an ester (a carboxylate) at the respective position. In some of these embodiments, one or more of R₇—R₉ is a sulfonyl as defined herein.

In some of any of the embodiments described herein, when one or more of R₇—R₉ is acyl, the acyl is such that R′ is an alkyl or alkaryl or aryl, each of which being optionally substituted by one or more amine substituents.

In some of these embodiments, R′ in the acyl is a substituted alkyl, and in some embodiments, R′ is substituted by hydroxy at the a position with respect to the carbonyl group, such that the acyl is a-hydroxy-acyl. In some of these embodiments, at least one of R₇—R₉ is a sulfonyl or R₃ is ORy and Ry is other than hydrogen (e.g., is an alkyl, an aryl, a cycloalkyl, or an alkaryl, each being optionally substituted) or R₂ and R₃ form together a dioxane ring as described herein in any of the respective embodiments.

In some embodiments, the a-hydroxy-acyl is further substituted by one or more amine groups, and is an amino-substituted a-hydroxy-acyl.

In some of the embodiments of an acyl group as described herein, the amine substituents can be, for example, at one or more of positions β, γ, δ, and/or co of the moiety R′, with respect to the acyl.

Exemplary amino-substituted a-hydroxy-acyls include, without limitation, the moiety (S)-4-amino-2-hydroxybutyryl, which is also referred to herein as AHB. According to some embodiments of the present invention, an alternative to the AHB moiety can be the α-hydroxy-β-aminopropionyl (AHP) moiety. Additional exemplary amino-substituted α-hydroxy-acyls include, but are not limited to, L-(−)-y-amino-α-hydroxybutyryl, L(−)-δ-amino-α-hydroxyvaleryl, L-(−)-β-benzyloxycarbonylamino-α-hydroxypropionyl, a L-(−)-δ-benzyloxycarbonylamino-α-hydroxyvaleryl

It is noted herein that according to some embodiments of the present invention, other moieties which involve a combination of carbonyl(s), hydroxyl(s) and amino group(s) along a lower alkyl exhibiting any stereochemistry, are contemplated as optional substituents in place of AHB and/or AHP, including, for example, 2-amino-3-hydroxybutanoyl, 3-amino hydroxypentanoyl, 5-amino-3-hydroxyhexanoyl and the likes.

In some of any of the embodiments described herein, one or more of R₄—R₆ is other than ORz. In some of any of the embodiments described herein, one or more of R₄—R₆ is hydrogen.

In some of any of the embodiments described herein, R₃ is hydrogen.

In some of any of the embodiments described herein, R₄ is hydrogen.

In some of any of the embodiments described herein, R₃ and R₄ are each hydrogen.

In some of any of the embodiments described herein, one or more of R₄—R₆ is ORz and Rz is independently a monosaccharide moiety or an oligosaccharide moiety, as defined herein, such that the compound is a pseudo-trisaccharide, a pseudo-tetrasaccharide, a pseudo-pentasaccharide, a pseudo hexasaccharide, etc.

Whenever one or more of R₄—R₆ is ORz and Rz is a monosaccharide moiety or an oligosaccharide moiety and one or more of R₄—R₆ is not ORz in which Rz is a monosaccharide moiety or an oligosaccharide moiety, the one or more of R₄—R₆ which is not ORz in which Rz is a monosaccharide moiety or an oligosaccharide moiety can be as described herein for any of the respective embodiments for R₄—R₆.

The term “monosaccharide”, as used herein and is well known in the art, refers to a simple form of a sugar that consists of a single saccharide molecule which cannot be further decomposed by hydrolysis. Most common examples of monosaccharides include glucose (dextrose), fructose, galactose, and ribose. Monosaccharides can be classified according to the number of carbon atoms of the carbohydrate, i.e., triose, having 3 carbon atoms such as glyceraldehyde and dihydroxyacetone; tetrose, having 4 carbon atoms such as erythrose, threose and erythrulose; pentose, having 5 carbon atoms such as arabinose, lyxose, ribose, xylose, ribulose and xylulose; hexose, having 6 carbon atoms such as allose, altrose, galactose, glucose, gulose, idose, mannose, talose, fructose, psicose, sorbose and tagatose; heptose, having 7 carbon atoms such as mannoheptulose, sedoheptulose; octose, having 8 carbon atoms such as 2-keto-3-deoxy-manno-octonate; nonose, having 9 carbon atoms such as sialose; and decose, having 10 carbon atoms. Monosaccharides are the building blocks of oligosaccharides like sucrose (common sugar) and other polysaccharides (such as cellulose and starch).

The term “oligosaccharide” as used herein refers to a compound that comprises two or more monosaccharide units, as these are defined herein, linked to one another via a glycosyl bond (—O—). Preferably, the oligosaccharide comprises 2-6 monosaccharides, more preferably the oligosaccharide comprises 2-4 monosaccharides and most preferably the oligosaccharide is a disaccharide moiety, having two monosaccharide units.

In some of any of the embodiments described herein, the monosaccharide is a pentose moiety, such as, for example, represented by Formula II. Alternatively, the monosaccharide moiety is hexose. Further alternatively, the monosaccharide moiety is other than pentose or hexose, for example, a hexose moiety as described in U.S. Pat. No. 3,897,412.

In some of any of the embodiments described herein, the monosaccharide moiety is a ribose, represented by Formula II:

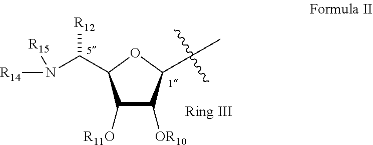

wherein the curved line denotes a position of attachment;

the dashed line indicates a stereo-configuration of position 5″ being an R configuration or an S configuration;

R₁₀ and R₁₁ are each independently selected from hydrogen, a substituted or unsubstituted alkyl, a substituted or unsubstituted cycloalkyl, a substituted or unsubstituted aryl, a substituted or unsubstituted alkaryl, and an acyl, as defined herein;

R₁₂ is selected from the group consisting of hydrogen, a substituted or unsubstituted alkyl, a substituted or unsubstituted cycloalkyl, and a substituted or unsubstituted aryl;

each of R₁₄ and R₁₅ is independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl, sulfonyl and a cell-permealizable group, or, alternatively, R₁₄ and R₁₅ form together a heterocyclic ring.

In some of any of the embodiments described herein, R₁₂ is hydrogen.

In some of any of the embodiments described herein, R₁₂ is other than hydrogen. In some of these embodiments, R₁₂ is alkyl, cycloalkyl or aryl, and in some embodiments, R₁₂ is alkyl, preferably a lower alkyl, for example, methyl. The alkyl, cycloalkyl or aryl can be substituted, as defined herein, or unsubstituted, preferably unsubstituted.

In some of any of the embodiments where one or more of R₄—R₆ is ORz and Rz is a monosaccharide moiety or an oligosaccharide moiety, one or more of the hydroxy groups in the monosaccharide or oligosaccharide moiety/moieties are substituted by an acyl, forming an ester (a carboxylate), as described herein in any of the respective embodiments.

In some of these embodiments, one or both of R₁₀ and R₁₁ is an acyl, forming an ester at the respective position(s), as described herein.

In some of any of the embodiments described herein, one of R₄—R₆ is ORz and Rz is a monosaccharide moiety such that the compound is a pseudo-trisaccharide.

In some of any of the embodiments described herein for a pseudo-trisaccharide, one or more, or all, of R₁₀ and R₁₁, can be an acyl, as described herein.

In some of any of the embodiments described herein for a pseudo-trisaccharide, one or more, or all, of R₄—R₆ are ORz, such that in one of R₄—R₆, Rz is a monosaccharide moiety, and in the others, Rz is as defined herein (e.g., hydrogen).

In some of any of the embodiments described herein, R₅ is ORz in which Rz is a monosaccharide moiety.

In some of these embodiments, the compound is represented by Formula B:

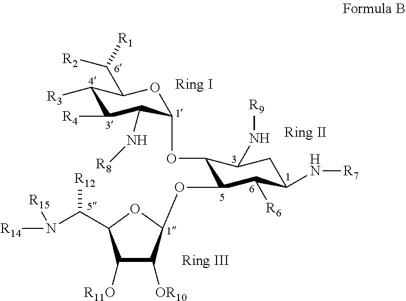

with the variables being as described herein for Formula A, including any combination thereof.

In some of any of the embodiments described herein for Formulae A and B, R₁ is hydrogen.

In some of any of the embodiments described herein for Formulae A and B, R₁ is other than hydrogen.

In some of any of the embodiments described herein for Formulae A and B, R₁ is alkyl, and in some embodiments it is a lower alkyl, of 1 to 4 carbon atoms, including, but not limited to, methyl, ethyl, propyl, butyl, isopropyl, and isobutyl.

In some of any of the embodiments described herein for Formulae A and B, R₁ is a non-substituted alkyl.

In some of any of the embodiments described herein for Formulae A and B, R₁ is methyl (e.g., a non-substituted methyl).

Alternatively, in some of any of the embodiments described herein for Formulae A and B, R₁ is cycloalkyl, including, but not limited to, cyclopropyl, cyclobutyl, cyclopentyl and cyclohexyl.

Further alternatively, in some of any of the embodiments described herein for Formulae A and B, R₁ is aryl, such as substituted or unsubstituted phenyl. Non-limiting examples include unsubstituted phenyl and toluene.

Further alternatively, in some of any of the embodiments described herein for Formulae A and B, R₁ is alkaryl, such as, for example, a substituted or unsubstituted benzyl.

In some of any of the embodiments described herein for Formulae A and B, R₁ is alkyl, alkenyl or alkynyl, each being substituted or unsubstituted.

In some of any of the embodiments described herein for Formulae A and B, R₁ is or comprises an aryl which can be substituted or unsubstituted. In some embodiments of Formulae A and B, R₁ is an unsubstituted aryl and can be, as non-limiting examples, phenyl, 1-anthryl, 1-naphthyl, 2-naphthyl, 2-phenanthryl or 9-phenanthryl.

In some embodiments of Formulae A and B, R₁ is a substituted aryl, and can be, as non-limiting examples, 2-(N-ethylamino)phenyl, 2-(N-hexylamino)phenyl, 2-(N-methylamino)phenyl, 2,4-dimethoxyphenyl, 2-acetamidophenyl, 2-aminophenyl, 2-carboxyphenyl, 2-chlorophenyl, 2-ethoxyphenyl, 2-fluorophenyl, 2-hydroxymethylphenyl, 2-hydroxyphenyl, 2-hydroxyphenyl, 2-methoxycarbonylphenyl, 2-methoxyphenyl, 2-methylphenyl, 2-N,N-dimethylaminophenyl, 2-trifluoromethylphenyl, 3-(N,N-dibutylamino)phenyl, 3-(N,N-diethylamino)phenyl, 3,4,5-trimethoxyphenyl, 3,4-dichlorophenyl, 3,4-dimethoxyphenyl, 3,5-dimethoxyphenyl, 3-aminophenyl, 3-biphenylyl, 3-carboxyphenyl, 3-chloro-4-methoxyphenyl, 3-chlorophenyl, 3-ethoxycarbonylphenyl, 3-ethoxyphenyl, 3-fluorophenyl, 3-hydroxymethylphenyl, 3-hydroxyphenyl, 3-isoamyloxyphenyl, 3-isobutoxyphenyl, 3-isopropoxyphenyl, 3-methoxyphenyl, 3-methylphenyl, 3-N,N-dimethylaminophenyl, 3-tolyl, 3-trifluoromethylphenyl, 4-(benzyloxy)phenyl, 4-(isopropoxycarbonyl)phenyl, 4-(N,N-diethylamino)phenyl, 4-(N,N-dihexylamino)phenyl, 4-(N,N-diisopropylamino)phenyl, 4-(N,N-dimethylamino)phenyl, 4-(N,N-di-n-pentylamino)phenyl, 4-(n-hexyloxycarbonyl)phenyl, 4-(N-m ethylamino)phenyl, 4-(trifluoromethyl)phenyl, 4-aminophenyl, 4-benzyloxyphenyl, 4-biphenylyl, 4-butoxyphenyl, 4-butyramidophenyl, 4-carboxyphenyl, 4-chlorophenyl, 4-ethoxycarbonylphenyl, 4-hexanamidophenyl, 4-hydroxymethylphenyl, 4-hydroxyphenyl, 4-iodophenyl, 4-isobutylphenyl, 4-isobutyramidophenyl, 4-isopropoxyphenyl, 4-isopropylphenyl, 4-methoxyphenyl, 4-methylphenyl, 4-n-hexanamidophenyl, 4-n-hexyloxyphenyl, 4-n-hexylphenyl, 4-nitrophenyl, 4-nitrophenyl, 4-propionamidophenyl, 4-tolyl, 4-trifluoromethylphenyl or 4-valeroyloxycarbonylphenyl .

In some of any of the embodiments described herein for Formulae A and B, R₁ is or comprises a substituted or unsubstituted heteroaryl, and can be, as non-limiting examples, 2-anthryl, 2-furyl, 2-indolyl, 2-naphthyl, 2-pyridyl, 2-pyrimidyl, 2-pyrryl, 2-quinolyl, 2-thienyl, 3-furyl, 3-indolyl, 3-thienyl, 4-imidazolyl, 4-pyridyl, 4-pyrimidyl, 4-quinolyl, 5-methyl -2-thienyl and 6-chloro-3-pyridyl.

In some of any of the embodiments described herein for Formulae A and B, R₁ is or comprises an amine, as defined herein, and can be, as non-limiting examples, —NH₂, —NHCH₃, —N(CH₃)₂, —NH—CH₂—CH₂—NH₂, —NH-CH₂—CH₂—OH and —NH—CH₂—CH(OCH₃)₂. In some of any of the embodiments described herein for Formula III or IIIa, R₁ is a hydroxyalkyl, for example, hydroxym ethyl.

In some of any of the embodiments described herein for Formulae A and B, R₁, comprises a hydroxy substituent, which forms a “diol-like” structure on Ring I when R₂ is ORx and Rx is hydrogen.

For example, in some embodiments, R₁ is configured such that a hydroxy substituent is 1-6, or 1-4, or 1-3, or 2, carbon atoms away from a hydroxy ORx group of R₂.

In some of any of the embodiments described herein for Formulae A and B, R₁ is a hydroxy-substituted alkyl, a hydroxy-substituted alkenyl, a hydroxy-substituted cycloalkyl or a hydroxy-substituted aryl.

In some of any of the embodiments described herein for Formulae A and B, R₁ is a hydroxy-substituted alkyl, or a hydroxy-substituted alkenyl, and the hydroxy substituent is a terminal substituent.

In some of any of the embodiments of R₁ in Formulae A and B, the alkyl or alkenyl are 1 to 10, or 1 to 8, preferably 1 to 6, or 1 to 4.

In some of any of the embodiments of R₁ in Formula A and B, R₁ is a hydroxyalkyl, wherein the alkyl can be further substituted or not.

In some of any of the embodiments described herein, R₁ is a hydroxymethyl.

In some of any of the embodiments of R₁ in Formulae A and B, the hydroxy-substituted alkyl, alkenyl, cycloalkyl or aryl can be further substituted or not, and can, for example, include 2 or more hydroxy groups.

In some of any of the embodiments described herein for Formulae A and B, R₂ is hydrogen. In some of any of the embodiments described herein for Formulae A and B, R₂ is ORx, and Rx is hydrogen.

In some of any of the embodiments described herein for Formulae A and B, R₂ is ORx, and Rx is other than hydrogen.

In some of any of the embodiments described herein for Formulae A and B, R₂ is ORx and Rx is an acyl, forming as ester at this position, as described herein.

In some embodiments, R₂ is ORx and Rx is an alkyl, preferably selected from the group consisting of methyl, ethyl and propyl.

In some of any of the embodiments described herein, R₂ is alkyl, and in some of these embodiments R₂ is a substituted alkyl, for example, an alkyl substituted by one or more amine groups (aminoalkyl).

In some of any of the embodiments described herein, R₂ is a substituted or unsubstituted alkyl, as defined herein, or a substituted or unsubstituted cycloalkyl, as defined herein.

In some of any of the embodiments described herein, R₂ is a substituted or unsubstituted aryl, as defined herein.

In some of any of the above embodiments pertaining to R₂, wherein R₂ does form a dioxane ring with R₃, at least one of R₇—R₉ is a sulfonyl and/or R₃ is ORy wherein Ry is other than hydrogen and/or at least one of R₇—R₉ is a sulfonyl.

According to some of any of the embodiments of Formulae A and B, one or both of the amine substituents at positions 1 (R₇), 3 (R₉), 2′ (R₈) or 5″ (R₁₄ and/or R₁₅, if present) of the aminoglycoside structure is modified, such that one or more of R₇—R₉ and of R₁₄ and R₁₅, if present, is not hydrogen.

Herein throughout, an amine which bears a substituent other than hydrogen is referred to herein as a “modified amine sub stituent” or simply as a “modified amine”.

According to some embodiments of the present invention, one or both of the amine substituents at positions 1 (R₇), 3 (R₉), 2′ (R₈) or 5″ (R₁₄ and/or R₁₅, if present) of the aminoglycoside structure is modified to include a hydrophobic moiety such as alkyl, cycloalkyl, alkaryl and/or aryl, or a group which is positively-charged at physiological pH and which can increase cell permeability of the compound (also referred to herein interchangeably as “cell-permealizable group” or “cell-permealizing group”), such as guanine or guanidine groups, as defined herein, or, alternatively, hydrazine, hidrazide, thiohydrazide, urea and thiourea.

In some of any of the embodiments described herein, the amine substituent at position 1 (Ring II) in Formula I, is a modified amine, as described herein, such that R₇ is other than hydrogen. Alternatively, or in addition, one or more of R₈ and R₉ is other than hydrogen.

In some of these embodiments, one or more of R₇—R₉ and of R₁₄ and R₁₅, if present, is independently an alkyl, a cell-permealizable group, as described herein, or an acyl as described herein in any of the respective embodiments.

Exemplary moieties represented by one or more of R₇—R₉ and of R₁₄ and R₁₅, if present, include, but are not limited to, hydrogen, (R/S)-4-amino-2-hydroxybutyryl (AHB), (R/S)-3-amino-2-hydroxypropionyl (AHP), 5-aminopentanoyl, 5-hydroxypentanoyl, formyl, —C(═O)—O-methyl, —C(═O)—O-ethyl, —C(═O)—O-benzyl, -(3-amino-α-hydroxypropionyl, -δ-amino-α-hydroxyvaleryl, -β-benzyloxycarbonyl amino-α-hydroxypropionyl, -δ- benzyloxycarbonylamino-α-hydroxyvaleryl, methylsulfonyl, phenylsulfonyl, benzoyl, propyl, isopropyl, —(CH₂)₂NH₂, —(CH₂)₃NH₂, —CH₂CH(NH₂)CH₃, —(CH₂)₄NH₂, —(CH₂)₅NH₂, —(CH₂)₂NH— ethyl, —(CH₂)₂NH(CH₂)₂NH₂, —(CH₂)₃NH(CH₂)₃NH₂, —(CH₂)₃NH(CH₂)₄NH(CH₂)₃NH₂, —CH(—NH₂)CH₂(OH), —CH(—OH)CH₂(NH₂), —CH(—OH)—(CH₂)₂(NH₂), —CH(—NH₂)—(CH₂)₂(OH), —CH(—CH₂NH₂)—(CH₂OH), —(CH₂)₄NH(CH₂)₃NH₂, —(CH₂)₂NH(CH₂)₂NH(CH₂)₂NH₂, —(CH₂)₂N(CH₂CH₂NH₂)₂, —CH₂—C(═O)NH₂, —CH(CH₃)—C(═O)NH₂, —CH₂-phenyl, —CH(i-propyl)-C(═O)NH₂, —CH(benzyl)-C(═O)NH₂, —(CH₂)₂OH, —(CH₂)₃OH and —CH(CH₂OH)₂.

In some of any of the embodiments described herein, R₇ is hydrogen, (R/S)-4-amino-2-hydroxybutyryl (AHB), (R/S)-3-amino-2-hydroxypropionyl, 5-aminopentanoyl, 5-hydroxypentanoyl, formyl, —C(═O)—O-methyl, —C(═O)—O-ethyl, —C(═O)—O-benzyl, -β-amino-α-hydroxypropionyl, -6-amino-α-hydroxyvaleryl, -β-benzyloxycarbonyl amino-α- hydroxypropionyl, -δ-benzyloxycarbonylamino-α-hydroxyvaleryl, methylsulfonyl, phenylsulfonyl, benzoyl, propyl, isopropyl, —(CH₂)₂NH₂, —(CH₂)₃NH₂, —CH₂CH(NH₂)CH₃, —(CH₂)₄NH₂, —(CH₂)₅NH₂, —(CH₂)₂NH—ethyl, —(CH₂)₂NH(CH₂)₂NH₂, —(CH₂)₃NH(CH₂)₃NH₂, —(CH₂)₃NH(CH₂)₄NH(CH₂)₃NH₂, —CH(—NH₂)CH₂(OH), —CH(—OH)CH₂(NH₂), —CH(—OH)—(CH₂)₂(NH₂), —CH(—NH₂)—(CH₂)₂(OH), —CH(—CH₂NH₂)—(CH₂OH), —(CH₂)₄NH(CH₂)₃NH₂, —(CH₂)₂NH(CH₂)₂NH(CH₂)₂NH₂, —(CH₂)₂N(CH₂CH₂NH₂)₂, —CH₂—C(O)NH₂, —CH(CH₃)—C(═O)NH₂, —CH₂-phenyl, —CH(i-propyl)-C(═O)NH₂, —CH(benzyl)-C(═O)NH₂, —(CH₂)₂₀H, —(CH₂)₃₀H or —CH(CH₂OH)₂.

In some of any of the embodiments described herein, one or both of R₈ and R₉ is independently hydrogen, (R/S)-4-amino-2-hydroxybutyryl (AHB), (R/S)-3-amino-2-hydroxypropionate (AHP), (R/S)-3-amino-2-hydroxypropionyl, 5-aminopentanoyl, 5-hydroxypentanoyl, formyl, —COO-methyl, —COO-ethyl, —COOO-benzyl, -β-amino-α-hydroxypropionyl, -6-amino-α-hydroxyvaleryl, -β-benzyloxycarbonylamino-α-hydroxypropionyl, -6-benzyloxycarbonylamino-α-hydroxyvaleryl, methyl sulfonyl, phenylsulfonyl, benzoyl, propyl, isopropyl, —(CH₂)₂NH₂, —(CH₂)₃NH₂, —CH₂CH(NH₂)CH₃, —(CH₂)₄NH₂, —(CH₂)₅NH₂, —(CH₂)₂NH—ethyl, —(CH₂)₂NH(CH₂)₂NH₂, —(CH₂)₃NH(CH₂)₃NH₂, —(CH₂)₃NH(CH₂)₄NH(CH₂)₃NH₂, —CH(—NH₂) CH₂(OH), —CH(—OH)CH₂(NH₂), —CH(—OH)—(CH₂)₂(NH₂), —CH(—NH₂)—(CH₂)₂(OH), —CH(—CH₂NH₂)—(CH₂OH), —(CH₂)₄NH(CH₂)₃NH₂, —(CH₂)₂NH(CH₂)₂NH(CH₂)₂NH₂, —(CH₂)₂N(CH₂CH₂NH₂)₂, —CH₂—C(═O)NH₂, —CH(CH₃)—C(═O)NH₂, —CH₂-phenyl, —CH(i-propyl)-C(═O)NH₂, —CH(benzyl)-C(═O)NH₂, —(CH₂)₂OH, —(CH₂)₃OH or —CH(CH₂OH)₂.

In some of any of the embodiments described herein, an amino-substituted alpha-hydroxy acyl is (S)-4-amino-2-hydroxybutyryl (AHB).

In some of any of the embodiments described herein, one or more R₇—R₉ and R₁₄ and R₁₅, if present, is a cell-permealizable group as defined herein, and in some embodiments, it is a guanidyl, as defined herein.

In some of any of the embodiments described herein, one or more R₇—R₉ and R₁₄ and R₁₅, if present, is a hydrophobic moiety such as alkyl, cycloalkyl, alkaryl and/or aryl.

In some of any of the embodiments described herein, one or more R₇—R₉ and R₁₄ and R₁₅, if present, is an acyl, as defined herein for the respective embodiments, and in some of these embodiments, the acyl can independently be an amino-substituted alpha-hydroxy acyl, as defined herein.

In some of the embodiments where one or more R₇—R₉ and R₁₄ and R₁₅, if present, is alkyl, the alkyl can be, for example, a lower alkyl, of 1-4 carbon atoms, such as, but not limited to, methyl, ethyl, propyl, butyl, isopropyl, and isobutyl, each being optionally substituted, as described herein.

In some of these embodiments, the alkyl is independently a non-substituted alkyl, such as, but not limited to, ethyl, propyl and isopropyl.

In some of these embodiments, the alkyl is independently a substituted methyl, such as, but not limited to, an alkaryl such as benzyl.

Alternatively, one or more R₇—R₉ and R₁₄ and R₁₅, if present, is independently a cycloalkyl, and the cycloalkyl can be, for example, cyclopropyl, cyclobutyl, cyclopentyl and cyclohexyl.

Further alternatively, one or more R₇—R₉ and R₁₄ and R₁₅, if present, is independently an aryl, and the aryl can be, for example, a substituted or unsubstituted phenyl. Non-limiting examples include unsubstituted phenyl and toluene.

In some of any of the embodiments described herein, the amine substituent at position 1 (R₇, Ring II) in Formula A or B, is a modified amine, as described herein, such that R₇ is other than hydrogen.

In some of these embodiments, R₇ can be alkyl, cycloalkyl, alkaryl, aryl, an acyl, or an amino-substituted a-hydroxy acyl, as defined herein, such as, for example, (S)-4-amino-2-hydroxybutyryl (AHB), or (S)-4-amino-2-hydroxypropionyl (AHP).

In some of the embodiments where R₇ is alkyl, the alkyl can be, for example, a lower alkyl, of 1-4 carbon atoms, such as, but not limited to, methyl, ethyl, propyl, butyl, isopropyl, and isobutyl, each being optionally substituted, as described herein.

In some of these embodiments, the alkyl is independently a non-substituted alkyl, such as, but not limited to, ethyl, propyl and isopropyl.

In some of these embodiments, the alkyl is independently a substituted methyl, such as, but not limited to, an alkaryl such as benzyl.

Alternatively, R₇ is cycloalkyl, and the cycloalkyl can be, for example, cyclopropyl, cyclobutyl, cyclopentyl and cyclohexyl.

Further alternatively, R₇ is aryl, and the aryl can be, for example, a substituted or unsubstituted phenyl. Non-limiting examples include unsubstituted phenyl and toluene.

In some of any of the embodiments described herein, R₇ is alkyl, cycloalkyl or aryl, as described herein.

In some of any of the embodiments described herein, R₇ is a cell-permealizable group, as defined herein, and in some embodiments, R₇ is guanidinyl.

In some of any of the embodiments of Formula B, one or both of R₁₄ and R₁₅ is other than hydrogen, such that an amine at position 5″ is a modified amine, as defined herein. In some of these embodiments, one or both of R₁₄ and R₁₅ is a cell-permealizable group such as, for example, a guanidine group. Alternatively, one or both of R₁₄ and R₁₅ is alkyl, cycloalkyl or aryl, as defined, for example, for any of the embodiments of R₇.

In some of any of the embodiments described herein, whenever none of R₇—R₉ and R₁₄ and R₁₅, if present, is sulfonyl, R₂ and R₃ form a dioxane ring as described herein.

In some of any of the embodiments described herein, whenever none of R₇—R₉ and R₁₄ and R₁₅, if present, is sulfonyl, R₃ is ORy and Ry is other than hydrogen, as described herein in any of the respective embodiments.

In some of any of the embodiments described herein throughout, whenever a variable is defined as an unsubstituted aryl, the unsubstituted aryl can be, for example, phenyl, 1-anthryl, 1-naphthyl, 2-naphthyl, 2-phenanthryl and/or 9-phenanthryl.

In some of any of the embodiments described herein, whenever a variable is defined as a substituted or unsubstituted heteroaryl, the heteroaryl can be, for example, 2-anthryl, 2-furyl, 2-indolyl, 2-naphthyl, 2-pyridyl, 2-pyrimidyl, 2-pyrryl, 2-quinolyl, 2-thienyl, 3-furyl, 3-indolyl, 3-thienyl, 4-imidazolyl, 4-pyridyl, 4-pyrimidyl, 4-quinolyl, 5-methyl-2-thienyl and/or 6-chloro-3-pyridyl.

In some of any of the embodiments described herein, whenever a variable is defined as a substituted aryl, the aryl can be, for example, 2-(N-ethylamino)phenyl, 2-(N-hexylamino)phenyl, 2-(N-methylamino)phenyl, 2,4-dimethoxyphenyl, 2-acetamidophenyl, 2-aminophenyl, 2-carboxyphenyl, 2-chlorophenyl, 2-ethoxyphenyl, 2-fluorophenyl, 2-hydroxymethylphenyl, 2-hydroxyphenyl, 2-hydroxyphenyl, 2-methoxycarbonylphenyl, 2-methoxyphenyl, 2-methylphenyl, 2-N,N-dimethylaminophenyl, 2-trifluoromethylphenyl, 3-(N,N-dibutylamino)phenyl, 3-(N,N-diethylamino)phenyl, 3,4,5-trimethoxyphenyl, 3,4-dichlorophenyl, 3,4-dimethoxyphenyl, 3,5-dimethoxyphenyl, 3-aminophenyl, 3-biphenylyl, 3-carboxyphenyl, 3-chloro-4-methoxyphenyl, 3-chlorophenyl, 3-ethoxycarbonylphenyl, 3-ethoxyphenyl, 3-fluorophenyl, 3-hydroxymethylphenyl, 3-hydroxyphenyl, 3-isoamyloxyphenyl, 3-isobutoxyphenyl, 3-isopropoxyphenyl, 3-methoxyphenyl, 3-methylphenyl, 3-N,N-dimethylaminophenyl, 3-tolyl, 3-trifluoromethylphenyl, 4-(benzyloxy)phenyl, 4-(isopropoxycarbonyl)phenyl, 4-(N,N-diethylamino)phenyl, 4-(N,N-dihexylamino)phenyl, 4-(N,N-diisopropylamino)phenyl, 4-(N,N-dimethylamino)phenyl, 4-(N,N-di-n-pentylamino)phenyl, 4-(n-hexyloxycarbonyl)phenyl, 4-(N-methylamino)phenyl, 4-(trifluoromethyl)phenyl, 4-aminophenyl, 4-benzyloxyphenyl, 4-biphenylyl, 4-butoxyphenyl, 4-butyramidophenyl, 4-carboxyphenyl, 4-chlorophenyl, 4-ethoxycarbonylphenyl, 4-hexanamidophenyl, 4-hydroxymethylphenyl, 4-hydroxyphenyl, 4-iodophenyl, 4-isobutylphenyl, 4-isobutyramidophenyl, 4-isopropoxyphenyl, 4-isopropylphenyl, 4-methoxyphenyl, 4-methylphenyl, 4-n-hexanamidophenyl, 4-n-hexyloxyphenyl, 4-n-hexylphenyl, 4-nitrophenyl, 4-nitrophenyl, 4-propionamidophenyl, 4-tolyl, 4-trifluoromethylphenyl and/or 4-valeroyloxycarbonylphenyl.

Sulfonyl-Containing Compounds:

According to some of any of the embodiments described herein, there are provided compounds represented by general Formula A or B, wherein at least one of R₇—R₉ is a sulfonyl, as defined herein. The compounds according to these embodiments are also referred to herein as “sulfonyl-containing compounds”.

According to some of any of the embodiments described herein, the sulfonyl-containing compounds are collectively represented by general Formula I:

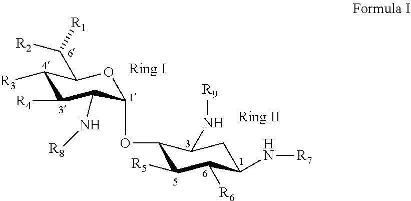

wherein:

the dashed line indicates a stereo-configuration of position 6′ being an R configuration or an S configuration, as described herein;

R₁ is selected from hydrogen, a substituted or unsubstituted alkyl, a substituted or unsubstituted cycloalkyl, and a substituted or unsubstituted aryl, and/or is as described herein in any of the respective embodiments for Formula A or B;

R₂ is selected from hydrogen, a substituted or unsubstituted alkyl and ORx, wherein Rx is selected from hydrogen, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl, and/or is as described herein for any of the respective embodiments for Formula A or B, or, alternatively, R₂ is ORx and forms together with R₃ a dioxane, as described herein in any of the respective embodiments;

R₃ is selected from hydrogen, a substituted or unsubstituted alkyl and ORy, wherein Ry is selected from hydrogen, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl, and/or is as described herein for any of the respective embodiments for Formula A or B, or, alternatively, R₃ is ORy and formed together with R₂ a dioxane;

R₄—R₆ are each independently selected from hydrogen, a substituted or unsubstituted alkyl, and ORz, wherein Rz is selected from hydrogen, a monosaccharide moiety, an oligosaccharide moiety, a substituted or unsubstituted alkyl, a substituted or unsubstituted cycloalkyl, a substituted to or unsubstituted aryl, a substituted or unsubstituted heteroaryl, a substituted or unsubstituted alkaryl, and an acyl, and/or is as described herein for any of the respective embodiments for Formula A or B; and

R₇—R₉ are each independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a sulfonyl, and/or is as described herein for any of the respective embodiments for Formula A or B,

provided that at least one of R₇—R₉ is a sulfonyl.

According to some of these embodiments, R₇ is the sulfonyl, and the compounds can be collectively represented by Formula Ia:

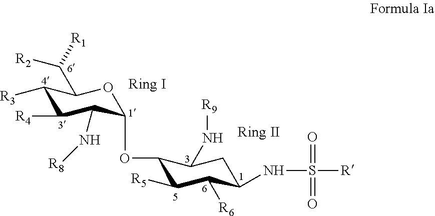

wherein:

R₁—R₆, R₈ and R₉ are as defined for Formula A, B or I; and

R′ is selected from hydrogen, a substituted or unsubstituted alkyl, a substituted or unsubstituted cycloalkyl, a substituted or unsubstituted alkaryl, and a substituted or unsubstituted aryl, as defined herein for a sulfonyl.

In some of any of the embodiments described herein, the sulfonyl is an alkyl sulfonyl, such that R′ is a substituted or unsubstituted alkyl. In some of these embodiments, the compound is represented by Formula Ia, and R′ in Formula Ia is a substituted or unsubstituted alkyl. In some embodiments, the sulfonyl is methyl sulfonyl, and R′ is methyl.

In some of any of the embodiments described herein, the sulfonyl is an aryl sulfonyl, such that R′ is a substituted or unsubstituted aryl. In some of these embodiments, the compound is represented by Formula Ia, and R′ in Formula Ia is a substituted or unsubstituted aryl (e.g., phenyl). In some embodiments, the sulfonyl is phenyl sulfonyl, and R′ is phenyl (e.g., unsubstituted phenyl).

Alternatively, and in any of the embodiments described herein for Formula I, R′ of the sulfonyl is an alkaryl such as a substituted or unsubstituted benzyl, or a cycloalkyl, or an alkenyl, or an alkynyl, or a heteroalicyclic, or a heteroaryl, each can be optionally substituted, as defined herein.

According to some of any of the embodiments described herein for Formula Ia, R₈ and R₉ are as described herein for Formula A.

According to some of any of the embodiments described herein for Formula Ia, R₈ and R₉ are each hydrogen.

According to some of any of the embodiments described herein for Formula I or Ia, R₂ is as described herein in any of the respective embodiments for Formula A or B.

According to some of any of the embodiments described herein for Formula I or Ia, R₂ is ORx, and Rx is as described herein in any of the respective embodiments for Formula A or B.

According to some of any of the embodiments described herein for Formula I or Ia, Rx is hydrogen, such that R₂ is hydroxy.

According to some of any of the embodiments described herein for Formula I or Ia, Rx is a substituted or unsubstituted alkyl, such that R₂ is alkoxy.

According to some of any of the embodiments described herein for Formula I or Ia, Rx is a substituted or unsubstituted aryl, such that R₂ is aryloxy.

According to some of any of the embodiments described herein for Formula I or Ia, R₃ is as described herein in any of the respective embodiments for Formula A or B.

According to some of any of the embodiments described herein for Formula I or Ia, R₃ is ORy, and Ry is as described herein in any of the respective embodiments for Formula A or B.

According to some of any of the embodiments described herein for Formula I or Ia, Ry is hydrogen, such that R₃ is hydroxy.

According to some of any of the embodiments described herein for Formula I or Ia, Ry is a substituted or unsubstituted alkyl, such that R₃ is alkoxy.

According to some of any of the embodiments described herein for Formula I or Ia, Ry is a substituted or unsubstituted aryl, such that R₃ is aryloxy.

According to some of any of the embodiments described herein for Formula I or Ia, R₂ and R₃ form together a dioxane, as described herein in any of the respective embodiments. In some embodiments, such compounds are represented by Formula I*b as described hereinafter.

According to some of any of the embodiments described herein for Formula I or Ia, R₁ is other than hydrogen, as described herein in any of the respective embodiments and any combination thereof, and in some of these embodiments, R₁ is an alkyl, for example, methyl.

According to some of any of the embodiments described herein for Formula I or Ia, each of R₄—R₆ is independently as described herein in any of the respective embodiments for Formulae A and B.

According to some of any of the embodiments described herein for Formula I or Ia, each of R₄—R₆ is independently ORz.

According to some of any of the embodiments described herein for Formula I or Ia, each of R₄—R₆ is ORz, and in each of the R₄—R₆ Rz is hydrogen, such that each of R₄—R₆ is hydroxy.

According to some of any of the embodiments described herein for Formula I or Ia, at least one of R₄—R₆ is ORz and Rz is a monosaccharide moiety or an oligosaccharide moiety, as described herein in any of the respective embodiments.

According to some of these embodiments, at least one of R₄—R₆ is ORz and Rz is a monosaccharide moiety represented by Formula II, as described herein in any of the respective embodiments.

According to some of any of the embodiments described herein for Formula I or Ia, R₅ is ORz and Rz is a monosaccharide moiety, as described herein in any of the respective embodiments. Such compounds can be collectively represented by general Formula III:

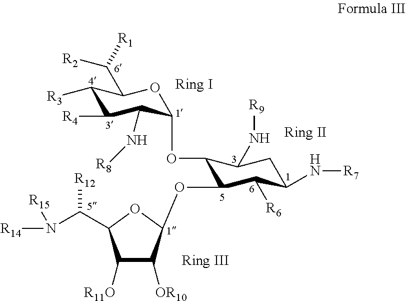

wherein:

R₁—R₄ and R₆—R₉ are each as defined herein in any of the respective embodiments and any combination thereof; and

R₁₀, R₁₁, R₁₂, R₁₄ and R₁₅ are each as defined for Formula II, in any of the respective embodiments and any combination thereof.

According to some of any of the embodiments described herein for Formula III, R₇ is the to sulfonyl, and such compounds can be collectively represented by Formula IIIa:

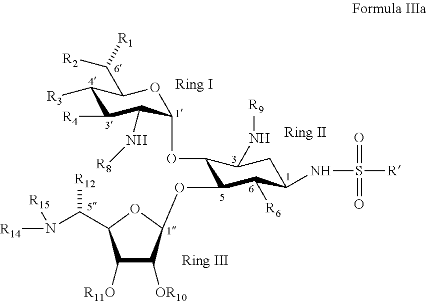

wherein:

R₁—R₄, R₆, R₈ and R₉ are as defined herein in any of the respective embodiments and any combination thereof;

R₁₀, R₁₁, R₁₂, R₁₄ and R₁₅ are as defined for Formula in any of the respective embodiments and any combination thereof; and

R′ is as defined herein in any of the respective embodiments for a sulfonyl, and any combination thereof.

According to some of any of the embodiments described herein for Formula III or IIIa, R₂ is ORx, and Rx is selected from hydrogen and a substituted or unsubstituted alkyl.

According to some of any of the embodiments described herein for Formula III or IIIa, R₃ is ORy, and Ry is selected from hydrogen and a substituted or unsubstituted alkyl.

According to some of any of the embodiments described herein for Formula III or IIIa, R₂ and R₃ form together a dioxane, as described herein in any of the respective embodiments. In some embodiments, such compounds are represented by Formula III*b as described hereinafter.

According to some of any of the embodiments described herein for Formula III or IIIa, R₄ and R₆ are each independently ORz, as defined herein in any of the respective embodiments.

According to some of any of the embodiments described herein for Formula III or IIIa, R₄ and R₆ are each ORz and Rz is hydrogen.

According to some of any of the embodiments described herein for Formula III or IIIa, R₈ and R₉ are each hydrogen. Alternatively, R₈ and R₉ are each independently as described herein in any of the embodiments relating to modified amines.

According to some of any of the embodiments described herein for Formula III or IIIa, R₁ is other than hydrogen, as described herein in any of the respective embodiments and any combination thereof, and in some of these embodiments, R₁ is an alkyl, for example, methyl.

According to some of any of the embodiments described herein for Formula IIIa, R₁₀, R₁₁, R₁₂, R₁₄ and R₁₅ are each hydrogen.

According to some of any of the embodiments described herein for Formula IIIa, R₁₀, R₁₁, R₁₄ and R₁₅ are each hydrogen and R₁₂ is selected from the group consisting of a substituted or unsubstituted alkyl, a substituted or unsubstituted cycloalkyl, and a substituted or unsubstituted aryl.

According to some of any of the embodiments described herein for Formula IIIa, R₁₂ is a substituted or unsubstituted alkyl.

According to some of any of the embodiments described herein, sulfonyl-containing compounds are represented by Formula IIIa, and according to some of these embodiments, R₁ is other than hydrogen, as described herein in any of the respective embodiments. In some of these embodiments, R₁ is alkyl, for example, methyl.

According to some of any of the embodiments described herein, sulfonyl-containing compounds are represented by Formula IIIa, and according to some of these embodiments, R₂ is ORx and Rx is hydrogen.

According to some of any of the embodiments described herein, sulfonyl-containing compounds are represented by Formula IIIa, and according to some of these embodiments, R₃ is ORy and Ry is hydrogen.

According to some of any of the embodiments described herein, sulfonyl-containing compounds are represented by Formula IIIa, and according to some of these embodiments, R₃ is ORy and Ry is an alkyl.

According to some of any of the embodiments described herein, sulfonyl-containing compounds are represented by Formula IIIa, and according to some of these embodiments, R₄ is ORz and Rz is hydrogen.

According to some of any of the embodiments described herein, sulfonyl-containing compounds are represented by Formula IIIa, and according to some of these embodiments, R₆ is ORz and Rz is hydrogen.

According to some of any of the embodiments described herein, sulfonyl-containing compounds are represented by Formula IIIa, and according to some of these embodiments, R₈ and R₉ are each hydrogen.

According to some of any of the embodiments described herein, sulfonyl-containing compounds are represented by Formula IIIa, and according to some of these embodiments, R₁₀, R₁₁, R₁₂, R₁₄ and R₁₅ are each hydrogen.

According to some of any of the embodiments described herein, sulfonyl-containing compounds are represented by Formula IIIa, and according to some of these embodiments, R₁₀,

R₁₁, R₁₄ and R₁₅ are each hydrogen and R₁₂ is other than hydrogen, as described herein in any of the respective embodiments. In some of these embodiments, R₁₂ is an alkyl, for example, methyl.

Exemplary compounds which are represented by Formula IIIa according to the present embodiments include, but are not limited to:

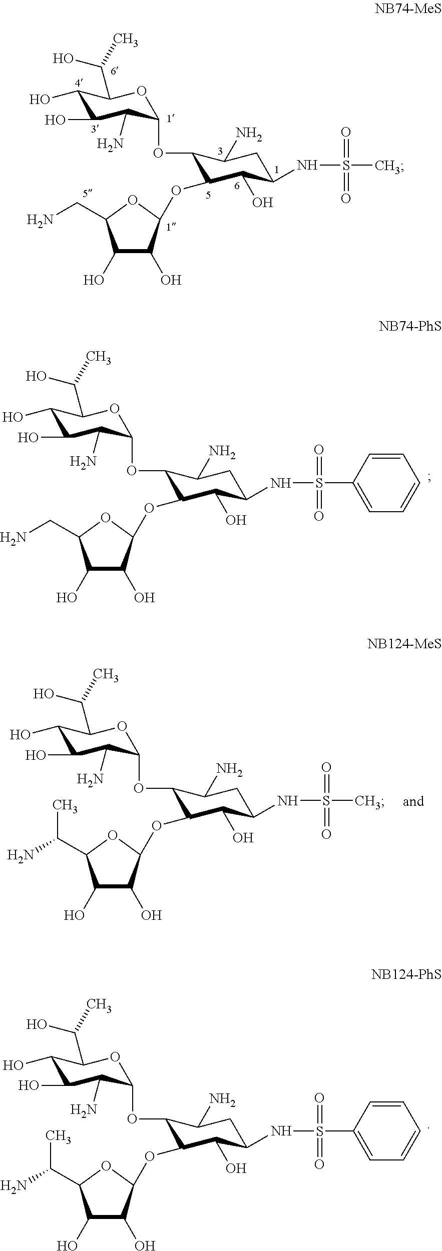

NB74-MeS is also referred to herein interchangeably as NB74-N1MeS or NB74-N1-MeS; NB74-PhS is also referred to herein interchangeably as NB74-N1PhS or NB74-N1-PhS; NB124-MeS is also referred to herein interchangeably as NB124-N1MeS or NB124-N1-MeS; and NB124-PhS is also referred to herein interchangeably as NB124-N1PhS or NB124-N1-PhS.

Dioxane-Containing Compounds:

According to some of any of the embodiments described herein, there are provided compounds represented by general Formula A or B, wherein R₂ and R₃ form together a dioxane ring, as defined herein in any of the respective embodiments. The compounds according to these embodiments are also referred to herein as “dioxane-containing compounds”, or as “C4′ and C6′-modified compounds” or as “C4′/C6′-modified compounds”.

According to some of any of the embodiments described herein, the dioxane-containing compounds are collectively represented by general Formula I*:

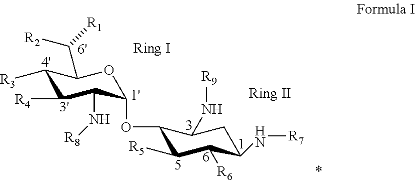

or a pharmaceutically acceptable salt thereof,

wherein:

the dashed line indicates a stereo-configuration of position 6′ being an R configuration or an S configuration (in case R₁ is other than hydrogen);

R₁ is selected from hydrogen, a substituted or unsubstituted alkyl, a substituted or unsubstituted cycloalkyl, and a substituted or unsubstituted aryl and/or is as described herein in any of the respective embodiments for Formula A or B;

R₂ is ORx, wherein Rx is selected from a substituted or unsubstituted alkyl, a substituted or unsubstituted cycloalkyl, a substituted or unsubstituted aryl, a substituted or unsubstituted heteroaryl, and a substituted or unsubstituted alkaryl;

R₃ is ORy, wherein Ry is selected from a substituted or unsubstituted alkyl, a substituted or unsubstituted cycloalkyl, a substituted or unsubstituted aryl, a substituted or unsubstituted heteroaryl, and a substituted or unsubstituted alkaryl;

R₄—R₆ are each independently selected from hydrogen, a substituted or unsubstituted alkyl, and ORz, wherein Rz is selected from hydrogen, a monosaccharide moiety, an oligosaccharide moiety, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl, and/or is as described herein in any of the respective embodiments for Formula A or B; and

R₇—R₉ are each independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a sulfonyl, and/or is as described herein in any of the respective embodiments for Formula A or B or Formula I or Ia,

and wherein ORx and ORy form together a dioxane, as described herein in any of the respective embodiments.

In some of any of the embodiments described herein for Formula I*, ORx and ORy form together a dioxane such that Rz and Ry are linked together to form a hydrocarbon as defined herein in any of the respective embodiments, linking the two oxygen atoms, such that the dioxane-containing compounds can be represented by Formula I**:

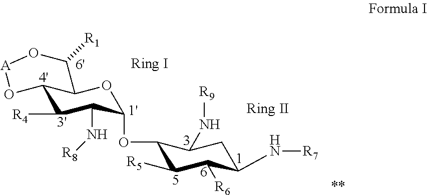

wherein A is a hydrocarbon as defined herein, and all other variables are as defined herein for Formula A, B or I*.

The number of carbon atoms in the backbone of the hydrocarbon determines the number of carbon atoms in the dioxane ring. For example, when the hydrocarbon is of 1 carbon atom in length (e.g., is a substituted or unsubstituted methylene), the dioxane ring is a 6-memebered ring. When the hydrocarbon is of 2 carbon atoms in length (e.g., is a substituted or unsubstituted ethylene), the to dioxane ring is a 7-memebered ring. When the hydrocarbon is of 3 carbon atoms in length (e.g., is a substituted or unsubstituted propylene), the dioxane ring is an 8-memebered ring, and so forth.

In some of any of the embodiments described herein for formula I*, the dioxane is a substituted or unsubstituted 1,3-dioxane.

In some of any of the embodiments described herein for formula I**, A is a substituted or unsubstituted methylene, and the dioxane is a substituted or unsubstituted 1,3-dioxane.

Compounds in which the dioxane is a 1,3-dioxane are also referred to herein as featuring a heterocyclic or cyclic acetal, and are collectively represented by Formula I*a:

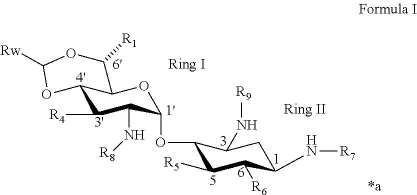

wherein:

R₁, R₄—R₆ and R₇—R₉ are as defined for Formula I*, A and B; and

Rw is selected from hydrogen, a substituted or unsubstituted alkyl, a substituted or unsubstituted cycloalkyl, a substituted or unsubstituted alkaryl, and a substituted or unsubstituted aryl.

In some of any of the embodiments described herein for Formula I*a, Rw is a substituted or unsubstituted alkyl, for example, methyl, ethyl, propyl, butyl, isopropyl, isobutyl, pentyl, etc., each being optionally substituted.

In some of any of the embodiments described herein for Formula I*a, Rw is an unsubstituted alkyl, as defined herein, and in some of these embodiments the alkyl is methyl.

In some of any of the embodiments described herein for Formula I*a, Rw is a substituted or unsubstituted aryl, for example, an unsubstituted phenyl or a substituted phenyl.

In some of any of the embodiments described herein for Formula I*a, Rw is an unsubstituted aryl, as defined herein, and in some of these embodiments the aryl is phenyl.

In some of any of the embodiments described herein for Formula I*a, Rw is a substituted aryl, as defined herein, and in some of these embodiments the aryl is phenyl.

When substituted, the phenyl can include one or more substituents. In some embodiments, a substituted phenyl is substituted at the para position with respect to the attachment point to the 1,3-dioxane moiety. In some embodiments, the phenyl substituent can be an alkyl or an alkoxy, as defined herein. In exemplary embodiments, Rw ispara-methoxyophenyl (PMP).

According to some of any of the embodiments described herein for Formula I*, I** or I*a, R₁ is other than hydrogen, as described herein in any of the respective embodiments and any combination thereof, and in some of these embodiments, R₁ is an alkyl, for example, methyl.

According to some of any of the embodiments described herein for Formula I*, I** or I*a, each of R₇—R₉ is independently as described herein in any of the respective embodiments for Formula A or B.

According to some of any of the embodiments described herein for Formula I*, I** or I*a, each of R₇—R₉ is hydrogen.

According to some of any of the embodiments described herein for Formula I*, I** or I*a, each of R₈ and R₉ is hydrogen, and R₇ is other than hydrogen, as described herein in any of the respective embodiments of Formula A.

According to some of any of the embodiments described herein for Formula I*, I** or I*a, R₈ and R₉ are each hydrogen, and wherein R₇ is selected from hydrogen, acyl, a substituted or unsubstituted alkyl, a substituted or unsubstituted cycloalkyl, a substituted or unsubstituted alkaryl, a substituted or unsubstituted aryl, an amino-substituted alpha-hydroxy acyl and a sulfonyl.

According to some of any of the embodiments described herein for Formula I*, I** or I*a, R₇ is acyl, as described herein in any of the respective embodiments.

According to some of any of the embodiments described herein for Formula I*, I** or I*a, R₇ is a sulfonyl, as described herein in any of the respective embodiments and such compounds can be collectively represented by Formula I*b:

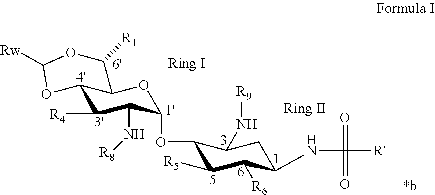

wherein:

Rw, R₁, R₄—R₆, R₈ and R₉ are as defined for Formula I*; and

R′ is selected from hydrogen, a substituted or unsubstituted alkyl, a substituted or unsubstituted cycloalkyl, a substituted or unsubstituted alkaryl, and a substituted or unsubstituted aryl, as described herein in any of the respective embodiments for a sulfonyl.

According to some of any of the embodiments described herein for Formula I*, I**, I*a or I*b, each of R₄—R₆ is independently as described herein in any of the respective embodiments for Formulae A and B.

According to some of any of the embodiments described herein for Formula I*, I**, I*a or I*b, each of R₄—R₆ is independently ORz.

According to some of any of the embodiments described herein for Formula I*, I**, I*a or I*b, each of R₄—R₆ is ORz, and in each of the R₄—R₆ Rz is hydrogen, such that each of R₄—R₆ is hydroxy.

According to some of any of the embodiments described herein for Formula I*, I**, I*a or I*b, at least one of R₄—R₆ is ORz and Rz is a monosaccharide moiety or an oligosaccharide moiety, as described herein in any of the respective embodiments.

According to some of any of the embodiments described herein for Formula I*, I**, I*a or I*b, at least one of R₄—R₆ is ORz and Rz is a monosaccharide moiety represented by Formula II, as described herein in any of the respective embodiments.

According to some of any of the embodiments described herein for Formula I*, I**, I*a or I*b, R₅ is ORz and Rz is a monosaccharide moiety, as described herein in any of the respective embodiments. Such compounds can be collectively represented by general Formula III*:

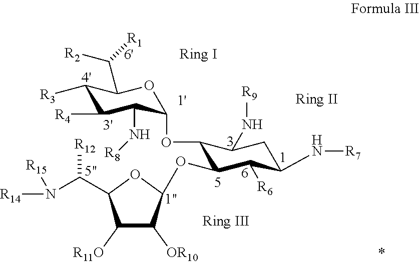

wherein:

R₁—R₄ and R₆—R₉ are each as defined for Formula I* or I*a or I*b, in any of the respective embodiments and any combination thereof; and

R₁₀, R₁₁, R₁₂, R₁₄ and R₁₅ are each as defined for Formula II in any of the respective embodiments and any combination thereof.

Compounds of Formula III* can also be represented by Formula III**:

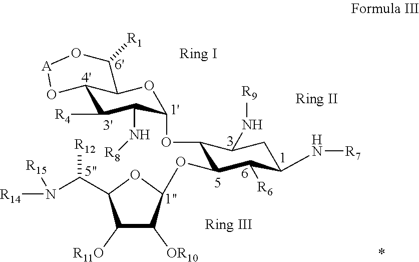

wherein:

R₁, R₄, R₆, and R₇—R₉ are each as defined for Formula I* or I*a or I*b, in any of the respective embodiments and any combination thereof;

A is a hydrocarbon as defined herein for Formula I**; and

R₁₀, R₁₁, R₁₂, R₁₄ and R₁₅ are each as defined for Formula II in any of the respective embodiments and any combination thereof.

According to some of any of the embodiments described herein, the dioxane is a substituted or unsubstituted 1,3-dioxane, as described herein in any of the respective embodiments, and in some of these embodiments, the compounds can be collectively represented by Formula III*a:

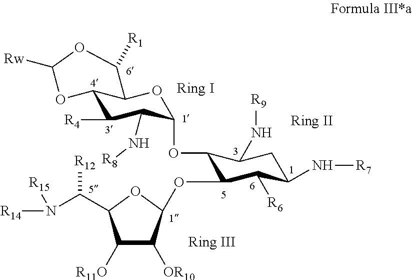

wherein:

R₁, R₄, R₆, and R₇—R₉ are as defined for Formula I* or I*a or I*b;

R₁₀, R₁₁, R₁₂, R₁₄ and R₁₅ are as defined for Formula II; and

Rw is selected from hydrogen, a substituted or unsubstituted alkyl, a substituted or unsubstituted cycloalkyl, a substituted or unsubstituted alkaryl, and a substituted or unsubstituted aryl, as described herein in any of the respective embodiments and any combination thereof.

According to some of any of the embodiments described herein for Formula III*, III*** and III*a, R₇ is a sulfonyl, and such compounds can be collectively represented by Formula III*b:

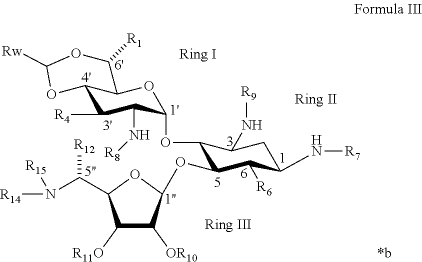

wherein:

Rw, R₁, R₄, R₆, R₈ and R₉ are as defined for Formula III*a;

R₁₀, R₁₁, R₁₂, R₁₄ and R₁₅ are as defined for Formula II; and

R′ is selected from hydrogen, a substituted or unsubstituted alkyl, a substituted or unsubstituted cycloalkyl, a substituted or unsubstituted alkaryl, and a substituted or unsubstituted aryl, as described herein in any of the respective embodiments and any combination thereof.

According to some of any of the embodiments described herein for Formula III*, III**, III*a or III*b, R₄ and R₆ are each independently ORz, as defined herein in any of the respective embodiments.

According to some of any of the embodiments described herein for Formula III*, III**, III*a or III*b, R₄ and R₆ are each ORz and Rz is hydrogen.

According to some of any of the embodiments described herein for Formula III*, III**, III*a or III*b, R₈ and R₉ are each hydrogen. Alternatively, R₈ and R₉ are each independently as described herein in any of the embodiments relating to modified amines.

According to some of any of the embodiments described herein for Formula III*, III**, or III*a, R₇ is hydrogen, or acyl, or is as described herein in any of the embodiments relating to modified amines.

According to some of any of the embodiments described herein for Formula III*, III**, III*a or III*b, R₁ is other than hydrogen, as described herein in any of the respective embodiments and any combination thereof, and in some of these embodiments, R₁ is an alkyl, for example, methyl.

According to some of any of the embodiments described herein for Formula III*, III**, III*a or III*b, R₁₀, R₁₁, R₁₂, R₁₄ and R₁₅ are each hydrogen.

According to some of any of the embodiments described herein for Formula III*, III**, III*a or III*b, R₁₀, R₁₁, R₁₄ and R₁₅ are each hydrogen and Rig is selected from the group consisting of a substituted or unsubstituted alkyl, a substituted or unsubstituted cycloalkyl, and a substituted or unsubstituted aryl.

According to some of any of the embodiments described herein for Formula III*, III**, III*a or III*b, Rig is a substituted or unsubstituted alkyl.

According to some of any of the embodiments described herein, dioxane-containing compounds are represented by Formula III*a, and according to some of these embodiments, R₁ is other than hydrogen, as described herein in any of the respective embodiments. In some of these embodiments, R₁ is alkyl, for example, methyl.

According to some of any of the embodiments described herein, dioxane-containing compounds are represented by Formula III*a, and according to some of these embodiments, R₇ is an acyl.

According to some of any of the embodiments described herein, dioxane-containing compounds are represented by Formula III*a, and according to some of these embodiments, R₇ is a sulfonyl.

According to some of any of the embodiments described herein, dioxane-containing compounds are represented by Formula III*a, and according to some of these embodiments, R₄ is ORz and Rz is hydrogen.

According to some of any of the embodiments described herein, dioxane-containing compounds are represented by Formula III*a, and according to some of these embodiments, R₆ is ORz and Rz is hydrogen.

According to some of any of the embodiments described herein, dioxane-containing compounds are represented by Formula III*a, and according to some of these embodiments, R₈ and R₉ are each hydrogen.

According to some of any of the embodiments described herein, dioxane-containing compounds are represented by Formula III*a, and according to some of these embodiments, R₁₀, R₁₁, R₁₂, R₁₄ and R₁₅ are each hydrogen.

According to some of any of the embodiments described herein, dioxane-containing compounds are represented by Formula III*a, and according to some of these embodiments, R₁₀, R₁₁, R₁₄ and R₁₅ are each hydrogen and R₁₂ is other than hydrogen, as described herein in any of the respective embodiments. In some of these embodiments, R₁₂ is an alkyl, for example, methyl.

Carboxyl-Containing Compounds:

According to some of any of the embodiments described herein, there are provided compounds which are generally represented by general Formula A or B, as defined herein in any of the respective embodiments, except for including a carboxyl-containing group at position C6′, and hence lacking the moieties represented by R₁ and R₂ in Formulae A and B. The compounds according to these embodiments are also referred to herein as “carboxyl-containing compounds”, or as “C6′-modified compounds”.

Herein throughout, the term “carboxyl” when used in the context of carboxyl-containing compounds, encompasses a —C(═O)—R₁₆ group and a —C(═S)—R₁₆, wherein R₁₆ can be hydrogen, such that the carboxyl-containing group is aldehyde or thioaldehyde; or wherein R₁₆ can be amine (substituted or unsubstituted), such that the carboxyl-containing group is amide or thioamide; R₁₆ can be ORq, with Rq being hydrogen, such that the carboxyl-containing group is carboxylic acid or thiocarboxylic acid; or with Rq being alkyl, cycloalkyl, aryl, alkaryl, heteroaryl and the like, as defined herein, each being optionally substituted, such that the carboxyl-containing group is a carboxylate (e.g., ester) or thiocarboxylate (e.g., thioester).

According to some of any of the embodiments described herein, the carboxyl-containing compounds are collectively represented by general Formula IV:

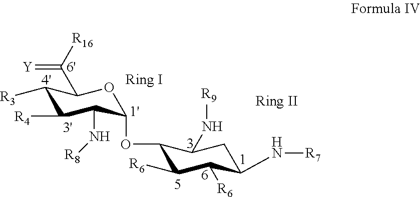

wherein:

Y is selected from oxygen and sulfur;

R₁₆ is selected from hydrogen, amine and ORq;

Rq is selected from hydrogen, a monosaccharide moiety, an oligosaccharide moiety, a substituted or unsubstituted alkyl, a substituted or unsubstituted cycloalkyl, a substituted or unsubstituted aryl, a substituted or unsubstituted heteroaryl, and a substituted or unsubstituted alkaryl, as defined herein in any of the respective embodiments;

R₃—R₆ are each independently selected from hydrogen, a substituted or unsubstituted alkyl, and ORz, wherein Rz is selected from hydrogen, a monosaccharide moiety, an oligosaccharide moiety, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl; and

R₇—R₉ are each independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a sulfonyl, as defined herein in any of the respective embodiments.

According to some of any of the embodiments described herein for Formula IV, Y is oxygen.

According to some of any of the embodiments described herein for Formula IV, R₁₆ is amine, —NR′R″, with R′ being as described herein and R″ being as described herein for R′.

According to some of any of the embodiments described herein for Formula IV, R₁₆ is ORq and Rq is hydrogen.

According to some of any of the embodiments described herein for Formula IV, each of R₇—R₉ is independently as described herein in any of the respective embodiments for Formula A or B.

According to some of any of the embodiments described herein for Formula IV, each of R₇—R₉ is hydrogen.

According to some of any of the embodiments described herein for Formula IV, each of R₈ and R₉ is hydrogen, and R₇ is other than hydrogen, as described herein in any of the respective embodiments of Formula A.

According to some of any of the embodiments described herein for Formula IV, R₈ and R₉ are each hydrogen, and wherein R₇ is selected from hydrogen, acyl, a substituted or unsubstituted alkyl, a substituted or unsubstituted cycloalkyl, a substituted or unsubstituted alkaryl, a substituted or unsubstituted aryl, an amino-substituted alpha-hydroxy acyl and a sulfonyl.

According to some of any of the embodiments described herein for Formula IV, R₇ is acyl, as described herein in any of the respective embodiments.

According to some of any of the embodiments described herein for Formula IV, R₇ is a sulfonyl, as described herein in any of the respective embodiments.

According to some of any of the embodiments described herein for Formula IV, each of R₄—R₆ is independently as described herein in any of the respective embodiments for Formulae A and B.

According to some of any of the embodiments described herein for Formula IV, each of R₄—R₆ is independently ORz.

According to some of any of the embodiments described herein for Formula IV, each of R₄—R₆ is ORz, and in each of the R₄—R₆ Rz is hydrogen, such that each of R₄—R₆ is hydroxy.

According to some of any of the embodiments described herein for Formula IV, R₃ is ORy, and Ry is hydrogen.

According to some of any of the embodiments described herein for Formula IV, R₃ is ORy, and Ry is as described herein in any of the respective embodiments for Formula A.

Exemplary compounds represented by Formula IV include NB160 and NB161 (see, for example, FIG. 10).

According to some of any of the embodiments described herein for Formula IV, at least one of R₄—R₆ is ORz and Rz is a monosaccharide moiety or an oligosaccharide moiety, as described herein in any of the respective embodiments.

According to some of any of the embodiments described herein for Formula IV, at least one of R₄—R₆ is ORz and Rz is a monosaccharide moiety represented by Formula II, as described herein in any of the respective embodiments.

According to some of any of the embodiments described herein for Formula IV, R₅ is ORz and Rz is a monosaccharide moiety, as described herein in any of the respective embodiments. Such compounds can be collectively represented by general Formula IVa:

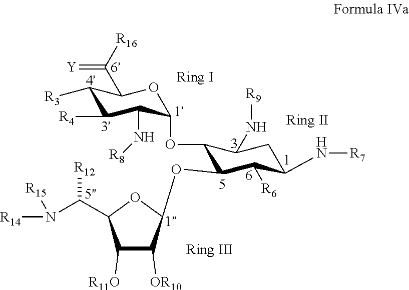

wherein:

the dashed line indicates a stereo-configuration of position 5″ being each independently to an R configuration or an S configuration;

Y, R₃, R₄ and R₆—R₉ are each as defined for Formula IV;

R₁₀ and R₁₁ are each independently selected from hydrogen, a substituted or unsubstituted alkyl, a substituted or unsubstituted cycloalkyl, a substituted or unsubstituted aryl, a substituted or unsubstituted alkaryl, and an acyl;

R₁₂ is selected from the group consisting of hydrogen, a substituted or unsubstituted alkyl, a substituted or unsubstituted cycloalkyl, and a substituted or unsubstituted aryl; and

each of R₁₄ and R₁₅ is independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl, a sulfonyl and a cell-permealizable group, or, alternatively, R₁₄ and R₁₅ form together a heterocyclic ring, as described herein in any of the respective embodiments for Formula B.

According to some of any of the embodiments described herein for Formula IVa, R₁₀, R₁₁, R₁₂, R₁₄ and R₁₅ are each hydrogen.

According to some of any of the embodiments described herein for Formula IVa, R₁₀, R₁₁, R₁₄ and R₁₅ are each hydrogen, and wherein R₁₂ is an alkyl.

Exemplary compounds represented by Formula IVa include NB162, NB163, NB164 and NB165 (see, FIG. 10).

General:

For any of the compounds described herein and represented by Formula A, B, I, Ia, I*, I**, I*a, I*b, III, IIIa, III*, III**, III*a, III*b, IV and IVa, it is to be noted that whenever optional substituents can be present in one or more of the carbon positions of the amino glycoside, for example, at position C6′, C4′, C3′, C2′, C1′, C3, C2, C1, C6, C5, and/or C1″, C2″, C3″, C4″, and C5″ (if present), and these substituents are not indicated, these substituents can each be hydrogen, or alternatively, each can be independently selected from alkyl, alkenyl, alkynyl, aryl, heteroaryl and cycloalkyl, each being substituted or unsubstituted, or, alternatively, each can be as defined herein for R₇—R₉.

Embodiments of the present invention further encompass compounds described herein and represented by Formula A, B, I, Ia, III, and IIIa, in which Ring I is an unsaturated ring and which can be referred to as “unsaturated Glucosamine (Ring I)-containing” compounds. Such compounds can be represented by Formula Ic or Id, as follows:

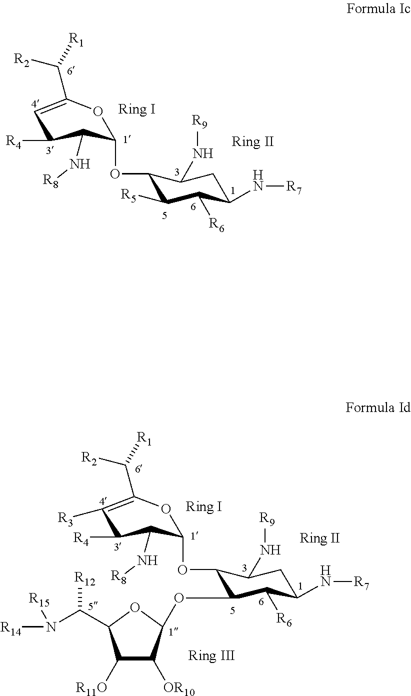

Wherein is all the variables are as defined herein for the respective variables of Formulae A, B, I, Ia, III and IIIa, respectively.

For any of the embodiments described herein, and any combination thereof, the compound may be in a form of a salt, for example, a pharmaceutically acceptable salt.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt comprising at least one basic (e.g., amine and/or guanidine) group of the compound which is in a positively charged form (e.g., wherein the basic group is protonated), in combination with at least one counter-ion, derived from the selected base, that forms a pharmaceutically acceptable salt.

The acid addition salts of the compounds described herein may therefore be complexes formed between one or more basic groups of the compound and one or more equivalents of an acid.

Depending on the stoichiometric proportions between the charged group(s) in the compound and the counter-ion in the salt, the acid additions salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

The present embodiments further encompass any enantiomers, diastereomers, prodrugs, solvates, hydrates and/or pharmaceutically acceptable salts of the compounds described herein.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an R- or an S-configuration and any combination, and compounds according to some embodiments of the present invention, can have any their chiral centers exhibit an R- or an S-configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

According to some of any of the embodiments described herein, a stereo-configuration of each of position 6′ and position 5″ (if present) is independently an R configuration or an S configuration.

According to some of any of the embodiments described herein, a stereo-configuration of position 6′ is an R configuration.

According to some of any of the embodiments described herein, a stereo-configuration of position 5″, if present, is an S configuration.

According to some of any of the embodiments described herein, a stereo-configuration of position 6′ is an R configuration and a stereo-configuration of position 5″, if preset, is an R configuration or an S configuration.

According to some of any of the embodiments described herein, a stereo-configuration of position 6′ is an R configuration and a stereo-configuration of position 5″, if present, is an S configuration.

In the structural formulae presented herein throughout, whenever a chiral carbon features a defined R- or an S-configuration, its chirality is represented by a triangle dashed or bolded line, as acceptable in the art, depending on the indicated stereoconfiguration, and is not specified. It is to be noted, however, that the present embodiments also encompass stereoconfigurations other than those reflected by triangle dashed or bolded lines shown. Whenever a chiral carbon features a configuration that can be either R-configuration or S-configuration, it is represented by a rectangular dashed line and is described as such. Chiral carbon atoms which can adopt R-configuration or S-configuration or a racemic mixture thereof are presented herein by a simple line or a curved (wavy) line.

In the structural formulae presented herein throughout, sub stituents of 6-memebered rings are shown as axial or equatorial, yet each of these substituents can adopt the other configuration and all such combinations are contemplated.

The term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of the active compound in vivo. An example, without limitation, of a prodrug would be a compound of the present invention, having one or more carboxylic acid moieties, which is administered as an ester (the “prodrug”). Such a prodrug is hydrolyzed in vivo, to thereby provide the free compound (the parent drug). The selected ester may affect both the solubility characteristics and the hydrolysis rate of the prodrug.

The term “solvate” refers to a complex of variable stoichiometry (e.g., di-, tri-, tetra-, penta-, hexa-, and so on), which is formed by a solute (the compound of the present invention) and a solvent, whereby the solvent does not interfere with the biological activity of the solute. Suitable solvents include, for example, ethanol, acetic acid and the like. The term “hydrate” refers to a solvate, as defined hereinabove, where the solvent is water.

According to some of any of the embodiments of the present invention, for Formula A, B, I, Ia, I*, I**, I*a, I*b, III, IIIa, III*, III**, III*a, and III*B, excluded from the scope of the present invention are compounds known in the art, including any of the documents cited in the Background section of the instant application, which are encompassed by these Formulae. Exemplary compounds which are excluded from the scope of the present embodiments include, but are not limited to, gentamicin, geneticin, fortimycin, apramycin, arbekacin, dibekacin, geneticin (G-418, G418), habekacin, kanamycin, Lividomycin, paromomycin, streptomycin and tobramycin.

The terms “hydroxyl” or “hydroxy”, as used herein, refer to an —OH group.

As used herein, the term “amine” describes a -NR′R″ group where each of R′ and R″ is independently hydrogen, alkyl, alkenyl, alkynyl, cycloalkyl, heteroalicyclic, aryl, heteroaryl, alkaryl, alkheteroaryl, or acyl, as these terms are defined herein. Alternatively, one or both of R′ and R″ can be, for example, hydroxy, alkoxy, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine.

As used herein, the term “alkyl” describes an aliphatic hydrocarbon including straight chain and branched chain groups. The alkyl may have 1 to 20 carbon atoms, or 1-10 carbon atoms, and may be branched or unbranched. According to some embodiments of the present invention, the alkyl is a low (or lower) alkyl, having 1-4 carbon atoms (namely, methyl, ethyl, propyl and butyl).

Whenever a numerical range; e.g., “1-10”, is stated herein, it implies that the group, in this case the alkyl group, may contain 1 carbon atom, 2 carbon atoms, 3 carbon atoms, etc., up to and including 10 carbon atoms. In some embodiments, the alkyl is a lower alkyl, including 1-6 or 1-4 carbon atoms.

An alkyl can be substituted or unsubstituted. When substituted, the substituent can be, for example, one or more of an alkyl (forming a branched alkyl), an alkenyl, an alkynyl, a cycloalkyl, an aryl, a heteroaryl, a heteroalicyclic, a halo, a trihaloalkyl, a hydroxy, an alkoxy and a hydroxyalkyl as these terms are defined hereinbelow. An alkyl substituted by aryl is also referred to herein as “alkaryl”, an example of which is benzyl.

Whenever “alkyl” is described, it can be replaced also by alkenyl or alkynyl. The term “alkyl” as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond, e.g., allyl, vinyl, 3-butenyl, 2-butenyl, 2-hexenyl and i-propenyl. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” refers to an all-carbon monocyclic or fused ring (i.e., rings which share an adjacent pair of carbon atoms), branched or unbranched group containing 3 or more carbon atoms where one or more of the rings does not have a completely conjugated pi-electron system, and may further be substituted or unsubstituted. Exemplary cycloalkyl groups include, for example, cyclopropyl, cyclobutyl, cyclopentyl, cyclohexyl, or cyclododecyl. The cycloalkyl can be substituted or unsubstituted. When substituted, the substituent can be, for example, one or more of an alkyl, an alkenyl, an alkynyl, a cycloalkyl, an aryl, a heteroaryl, a heteroalicyclic, a halo, a trihaloalkyl, a hydroxy, an alkoxy and a hydroxyalkyl as these terms are defined hereinbelow.

The term “aryl” describes an all-carbon monocyclic or fused-ring polycyclic (i.e., rings which share adjacent pairs of carbon atoms) groups having a completely conjugated pi-electron system. The aryl group may be unsubstituted or substituted by one or more substituents. When substituted, the substituent can be, for example, one or more of an alkyl, an alkenyl, an alkynyl, a cycloalkyl, an aryl, a heteroaryl, a heteroalicyclic, a halo, a trihaloalkyl, a hydroxy, an alkoxy and a hydroxyalkyl as these terms are defined hereinbelow.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. Representative examples are thiadiazole, pyridine, pyrrole, oxazole, indole, purine and the like.

The heteroaryl group may be unsubstituted or substituted by one or more substituents. When substituted, the substituent can be, for example, one or more of an alkyl, an alkenyl, an alkynyl, a cycloalkyl, an aryl, a heteroaryl, a heteroalicyclic, a halo, a trihaloalkyl, a hydroxy, an alkoxy and a hydroxyalkyl as these terms are defined hereinbelow.

The term “heteroalicyclic”, as used herein, describes a monocyclic or fused ring group having in the ring(s) one or more atoms such as nitrogen, oxygen and sulfur. The rings may also have one or more double bonds. However, the rings do not have a completely conjugated pi-electron system. Representative examples are morpholine, piperidine, piperazine, tetrahydrofurane, tetrahydropyrane and the like. The heteroalicyclic may be substituted or unsubstituted. When substituted, the substituent can be, for example, one or more of an alkyl, an alkenyl, an alkynyl, a cycloalkyl, an aryl, a heteroaryl, a heteroalicyclic, a halo, a trihaloalkyl, a hydroxy, an alkoxy and a hydroxyalkyl as these terms are defined hereinbelow.

The term “halide”, as used herein, refers to the anion of a halo atom, i.e. F⁻, Cl⁻, Br⁻ and I⁻.

The term “halo” refers to F, Cl, Br and I atoms as substituents.

The term “alkoxide” refers to an R′—O⁻ anion, wherein R′ is as defined hereinabove.

The term “alkoxy” refers to an —OR′ group, wherein R′ is alkyl or cycloalkyl, as defined herein.

The term “aryloxy” refers to an —OR′ group, wherein R′ is aryl, as defined herein.

The term “heteroaryloxy” refers to an —OR′ group, wherein R′ is heteroaryl, as defined herein.

The term “thioalkoxy” refers to an —SR′ group, wherein R′ is alkyl or cycloalkyl, as defined herein.

The term “thioaryloxy” refers to an —SR′ group, wherein R′ is aryl, as defined herein.

The term “thioheteroaryloxy” refers to an —SR′ group, wherein R′ is heteroaryl, as defined herein.

The term “hydroxyalkyl,” as used herein, refers to an alkyl group, as defined herein, substituted with one or more hydroxy group(s), e.g., hydroxymethyl, 2-hydroxyethyl and 4-hydroxypentyl.

The term “aminoalkyl,” as used herein, refers to an alkyl group, as defined herein, substituted with one or more amino group(s).

The term “alkoxyalkyl,” as used herein, refers to an alkyl group substituted with one or more alkoxy group(s), e.g., methoxymethyl, 2-methoxyethyl, 4-ethoxybutyl, n-propoxyethyl and t-butyl ethyl.

The term “trihaloalkyl” refers to —CX₃, wherein X is halo, as defined herein. An exemplary haloalkyl is CF₃.

A “guanidino” or “guanidine” or “guanidinyl” or “guanidyl” group refers to an —RaNC(═NRd)—NRbRc group, where each of Ra, Rb, Rc and Rd can each be as defined herein for R′ and R″.

A “guanyl” or “guanine” group refers to an RaRbNC(═NRd)- group, where Ra, Rb and Rd are each as defined herein for R′ and R″.

In some of any of the embodiments described herein, the guanidine group is —NH—C(═NH)—NH₂.

In some of any of the embodiments described herein, the guanyl group is H₂N—C(═NH)— group.

Any one of the amine (including modified amine), guanidine and guanine groups described herein is presented as a free base form thereof, but is meant to encompass an ionized form thereof at physiological pH, and/or within a salt thereof, e.g., a pharmaceutically acceptable salt thereof, as described herein.

Whenever an alkyl, cycloalkyl, aryl, alkaryl, heteroaryl, heteroalicyclic, acyl and any other moiety as described herein is substituted, it includes one or more substituents, each can independently be, but are not limited to, hydroxy, alkoxy, thiohydroxy, thioalkoxy, aryloxy, thioaryloxy, alkaryl, alkenyl, alkynyl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide, as these terms are defined herein.

The term “cyano” describes a —C≡N group.

The term “nitro” describes an —NO₂ group.

The term “sulfate” describes a —O—S(═O)₂—OR′ end group, as this term is defined hereinabove, or an —O—S(═O)₂—O— linking group, as these phrases are defined hereinabove, where R′ is as defined hereinabove.

The term “thiosulfate” describes a —O—S(═S)(═O)—OR′ end group or a —O—S(═S)(═O)—O— linking group, as these phrases are defined hereinabove, where R′ is as defined hereinabove.

The term “sulfite” describes an —O—S(═O)—O—R′ end group or a —O—S(═O)—O— group linking group, as these phrases are defined hereinabove, where R′ is as defined hereinabove.

The term “thiosulfite” describes a —O—S(═S)—O—R′ end group or an —O—S(═S)—O— group linking group, as these phrases are defined hereinabove, where R′ is as defined hereinabove.

The term “sulfinate” describes a —S(═O)-OR′ end group or an —S(═O)—O— group linking group, as these phrases are defined hereinabove, where R′ is as defined hereinabove.

The term “sulfoxide” or “sulfinyl” describes a —S(═O)R′ end group or an —S(═O)— linking group, as these phrases are defined hereinabove, where R′ is as defined hereinabove.

The term “sulfonate” or “sulfonyl” describes a —S(═O)₂—R′ end group or an —S(═O)₂— linking group, as these phrases are defined hereinabove, where R′ is as defined herein.

The term “S-sulfonamide” describes a —S(═O)₂—NR′R″ end group or a —S(═O)₂—NR′— linking group, as these phrases are defined hereinabove, with R′ and R″ as defined herein.

The term “N-sulfonamide” describes an R′S(═O)₂—NR″— end group or a —S(═O)₂—NR′— linking group, as these phrases are defined hereinabove, where R′ and R″ are as defined herein.

The term “carbonyl” or “carbonate” as used herein, describes a —C(═O)—R′ end group or a —C(═O)— linking group, as these phrases are defined hereinabove, with R′ as defined herein.

The term “thiocarbonyl ” as used herein, describes a —C(═S)—R′ end group or a —C(═S)— linking group, as these phrases are defined hereinabove, with R′ as defined herein.

The term “oxo” as used herein, describes a (═O) group, wherein an oxygen atom is linked by a double bond to the atom (e.g., carbon atom) at the indicated position.

The term “thiooxo” as used herein, describes a (═S) group, wherein a sulfur atom is linked by a double bond to the atom (e.g., carbon atom) at the indicated position.

The term “oxime” describes a ═N—OH end group or a ═N—O— linking group, as these phrases are defined hereinabove.

The term “acyl halide” describes a —(C═O)R″″ group wherein R″″ is halide, as defined hereinabove.

The term “azo” or “diazo” describes an —N═NR′ end group or an —N═N— linking group, as these phrases are defined hereinabove, with R′ as defined hereinabove.

The term “azide” describes an —N₃ end group.

The term “carboxylate” as used herein encompasses C-carboxylate and O-carboxylate.

The term “C-carboxylate” describes a —C(═O)—OR′ end group or a —C(═O)—O— linking group, as these phrases are defined hereinabove, where R′ is as defined herein.

The term “O-carboxylate” describes a —OC(═O)R′ end group or a —OC(═O)— linking group, as these phrases are defined hereinabove, where R′ is as defined herein.

A carboxylate can be linear or cyclic. When cyclic, R′ and the carbon atom are linked together to form a ring, in C-carboxylate, and this group is also referred to as lactone. Alternatively, R′ and O are linked together to form a ring in O-carboxylate. Cyclic carboxylates can function as a linking group, for example, when an atom in the formed ring is linked to another group.

The term “thiocarboxylate” as used herein encompasses C-thiocarboxylate and O-thiocarboxylate.

The term “C-thiocarboxylate” describes a —C(═S)—OR′ end group or —-C(═S)—O— linking group, as these phrases are defined hereinabove, where R′ is as defined herein.

The term “O-thiocarboxylate” describes a —OC(═S)R′ end group or a —OC(═S)— linking group, as these phrases are defined hereinabove, where R′ is as defined herein.

A thiocarboxylate can be linear or cyclic. When cyclic, R′ and the carbon atom are linked together to form a ring, in C-thiocarboxylate, and this group is also referred to as thiolactone. Alternatively, R′ and O are linked together to form a ring in O-thiocarboxylate. Cyclic thiocarboxylates can function as a linking group, for example, when an atom in the formed ring is linked to another group.

The term “carbamate” as used herein encompasses N-carbamate and O-carbamate.

The term “N-carbamate” describes an R″OC(═O)—NR′— end group or a —OC(═O)—NR′— linking group, as these phrases are defined hereinabove, with R′ and R″ as defined herein.

The term “O-carbamate” describes an —OC(═O)—NR′R″ end group or an —OC(═O)—NR′— linking group, as these phrases are defined hereinabove, with R′ and R″ as defined herein.

A carbamate can be linear or cyclic. When cyclic, R′ and the carbon atom are linked together to form a ring, in O-carbamate. Alternatively, R′ and O are linked together to form a ring in N-carbamate. Cyclic carbamates can function as a linking group, for example, when an atom in the formed ring is linked to another group.

The term “carbamate” as used herein encompasses N-carbamate and O-carbamate..

The term “thiocarbamate” as used herein encompasses N-thiocarbamate and O-thiocarbamate.

The term “O-thiocarbamate” describes a —OC(═S)—NR′R″ end group or a —OC(═S)—NR′— linking group, as these phrases are defined hereinabove, with R′ and R″ as defined herein.

The term “N-thiocarbamate” describes an R″OC(═S)NR′— end group or a —OC(═S)NR′— linking group, as these phrases are defined hereinabove, with R′ and R″ as defined herein.

Thiocarbamates can be linear or cyclic, as described herein for carbamates.

The term “dithiocarbamate” as used herein encompasses S-dithiocarbamate and N-dithiocarbamate.

The term “S-dithiocarbamate” describes a —SC(═S)—NR′R″ end group or a —SC(═S)NR′— linking group, as these phrases are defined hereinabove, with R′ and R″ as defined herein.

The term “N-dithiocarbamate” describes an R″SC(═S)NR′— end group or a —SC(═S)NR′— linking group, as these phrases are defined hereinabove, with R′ and R″ as defined herein.

The term “urea”, which is also referred to herein as “ureido”, describes a —NR′C(═O)—NR″R′″ end group or a —NR′C(═O)—NR″— linking group, as these phrases are defined hereinabove, where R′ and R″ are as defined herein and R′″ is as defined herein for R′ and R″.

The term “thiourea”, which is also referred to herein as “thioureido”, describes a —NR′—C(═S)—NR″R′″ end group or a —NR′—C(═S)—NR″— linking group, with R′, R″ and R′″ as defined herein.

The term “amide” as used herein encompasses C-amide and N-amide.

The term “C-amide” describes a —C(═O)—NR′R″ end group or a —C(═O)—NR′— linking group, as these phrases are defined hereinabove, where R′ and R″ are as defined herein.

The term “N-amide” describes a R′C(═O)—NR″— end group or a R′C(═O)—N— linking group, as these phrases are defined hereinabove, where R′ and R″ are as defined herein.

The term “hydrazine” describes a —NR′—NR″R′″ end group or a —NR′—NR″— linking group, as these phrases are defined hereinabove, with R′, R″, and R′″ as defined herein.

As used herein, the term “hydrazide” describes a —C(═O)—NR′—NR″R′″ end group or a —C(═O)—NR′—NR″— linking group, as these phrases are defined hereinabove, where R′, R″ and R′− are as defined herein.

As used herein, the term “thiohydrazide” describes a —C(═S)—NR′—NR″R′″ end group or a -C(═S)-NR'-NR″- linking group, as these phrases are defined hereinabove, where R′, R″ and R′″ are as defined herein.

The Processes:

Further according to embodiments of the present invention, there are provided processes of preparing the compounds as described herein.

These processes are generally effected by providing a paromamine derivative and introducing thereto a desired modification to thereby obtain a pseudo-disaccharide compound as described herein.

Processes of preparing pseudo-trisaccharide compounds as described herein are generally effected by devising appropriate acceptor aminoglycoside molecules and corresponding donor molecules, as is known in the art of aminoglycosides.

Generally, the synthesis of pseudo-trisaccharide compounds according to some embodiments of the present invention is accomplished using suitable acceptor and donor molecules and reaction conditions which allow reacting a protected derivative of the donor and/or of the acceptor and removing the protecting group so as to obtain a desired pseudo-trisaccharide.

The term “acceptor” is used herein to describe the skeletal structure derived from paromamine which has an available (unprotected) hydroxyl group at position C3′, C4′, C6 or C5, preferably C5, which is reactive during a glycosylation reaction, and can accept a glycosyl.

The term “donor” is used herein to describe the glycosyl that reacts with the acceptor to form the final pseudo-trisaccharide compound.

The term “glycosyl”, as used herein, refers to a chemical group which is obtained by removing the hydroxyl group from the hemiacetal function of a monosaccharide.

The donors and acceptors are designed so as to form the desired compounds according to some embodiments of the present invention. The following describes some embodiments of this aspect of the present invention, presenting exemplary processes of preparing exemplary subsets of the compounds described herein. More detailed processes of preparing exemplary compounds according to some embodiments of the present invention, are presented in the Examples section that follows below and accompanying Figures.

The syntheses of pseudo-trisaccharide compounds according to some embodiments of the present invention, generally include (i) preparing an acceptor compound by selective protection of one or more hydroxyls and amines at selected positions present on the paromamine scaffold, leaving the selected position (e.g., C5) unprotected and therefore free to accept a donor (glycosyl) compound as defined herein; (ii) preparing a donor compound by selective protection of one or more hydroxyls and amines at selected positions present on the glycosyl, leaving one position unprotected and therefore free to couple with an acceptor compound as defined herein; (iii) subjecting the donor and the acceptor to a coupling reaction; and (iii) removing the protecting groups to thereby obtain the desired compound.

The phrase “protected group”, as used herein, refers to a group that is substituted or modified so as to block its functionality and protect it from reacting with other groups under the reaction conditions (e.g., a coupling reaction as described herein). A protected group is re-generated by removal of the substituent or by being re-modified.

When an “amino-protected group” or “hydroxyl-protected group” are used, it is meant that a protecting group is attached or used to modify the respective group so as to generate the protected group.

The phrase “protecting group”, as used herein, refers to a substituent or a modification that is commonly employed to block or protect a particular functionality while reacting other functional groups on the compound. The protecting group is selected so as to release the substituent or to be re-modified, to thereby generate the desired unprotected group.

For example, an “amino-protecting group” or “amine-protecting group” is a substituent attached to an amino group, or a modification of an amino group, that blocks or protects the amino functionality in the compound, and prevents it from participating in chemical reactions. The amino-protecting group is removed by removal of the substituent or by a modification that re-generates an amine group.

Suitable amino-protected groups include azide (azido), N-phthalimido, N-acetyl, N-trifluoroacetyl, N-t-butoxycarbonyl (BOC), N-benzyloxycarbonyl (CBz) and N-9-fluorenylmethylenoxycarbonyl (Fmoc).

A “hydroxyl-protecting group” or “hydroxyl-protecting group” refers to a substituent or a modification of a hydroxyl group that blocks or protects the hydroxyl functionality, and prevents it from participating in chemical reactions. The hydroxy-protecting group is removed by removal of the substituent or by a modification that re-generates a hydroxy group.

Suitable hydroxy protected groups include isopropylidene ketal and cyclohexanone dimethyl ketal (forming a 1,3-dioxane with two adjacent hydroxyl groups), 4-methoxy-1-methylbenzene (forming a 1,3-dioxane with two adjacent hydroxyl groups), O-acetyl, O-chloroacetyl, O-benzoyl and O-silyl (e.g., O-trimethylsilyl; O-TMS).

For a general description of protecting groups and their use, see T. W. Greene, Protective Groups in Organic Synthesis, John Wiley & Sons, New York, 1991.

According to some embodiments, the amino-protected groups include an azido (N₃—) and/or an N-phthalimido group, and the hydroxyl-protecting groups include O-acetyl (AcO—), O-benzoyl (BzO—), O-TMS (TMSO—) and/or O-chloroacetyl.

It is noted herein that when applicable, a “protected group” refers to a moiety in which one reactive function on a compound is protected or more than one reactive function are protected at the same time, such as in the case of two adjacent functionalities, e.g., two hydroxyl groups that can be protected at once by a isopropylidene ketal.

In some embodiments, the donor compound is a protected monosaccharide which can be represented by the general Formula II*, having a leaving group at position 1″ thereof, denoted L, and optionally a substituent R₁₂ at position 5″, as defined herein:

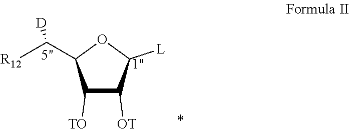

wherein:

L is a leaving group;

OT is a donor protected hydroxyl group;

R₁₂ is as defined herein for Formula Ib (the configuration at the 5″ position as presented in Formula III being a non-limiting example); and

D is a protected or unprotected form of NR₁₄R₁₅ as defined for Formulae III, IIIa, III*, III*a, III*b, III** and IVa, wherein when R₁₄ and R₁₅ in Formulae III, IIIa, III*, III*a, III*b, III** and IVa are both hydrogen, D is a donor protected amine group.

As used herein, the phrase “leaving group” describes a labile atom, group or chemical moiety that readily undergoes detachment from an organic molecule during a chemical reaction, while the detachment is typically facilitated by the relative stability of the leaving atom, group or moiety thereupon. Typically, any group that is the conjugate base of a strong acid can act as a leaving group. Representative examples of suitable leaving groups according to some of the present embodiments include, without limitation, trichloroacetimidate, acetate, tosylate, triflate, sulfonate, azide, halide, hydroxy, thiohydroxy, alkoxy, cyanate, thiocyanate, nitro and cyano.

According to some embodiments of the present invention, each of the donor hydroxyl-protecting groups is O-benzoyl and the donor amino-protecting group in either R₁₅ or R₁₇ is azido, although other protecting groups are contemplated.

It is to be noted that when one of R₁₄ and R₁₅ is other than hydrogen, it can be protected or unprotected. Typically, when one of R₆ and R₇ is guanine or guanidine, a protecting group suitable for the reaction conditions (e.g., of a coupling reaction with an acceptor) can be used. Optionally, the guanine or guanidine are unprotected. When one of R₁₄ and R₁₅ is an alkyl, aryl or cycloalkyl, typically D in Formula II* is an unprotected form of NR₁₄R₁₅.

The structure of the donor compound sets the absolute structure of Ring III in the resulting compound according to some embodiments of the present invention, namely the stereo-configuration of the 5″ position and the type of R₁₄, R₁₅ and R₁₂ in Formulae III, IIIa, III*, III*a, III*b, III** and IVa.

Exemplary acceptor molecules suitable for use in the preparation of the compounds described herein under Formula IIIa, are represented by Formula V:

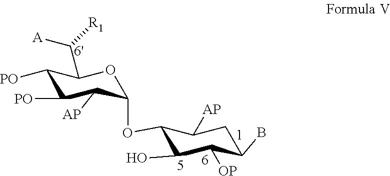

wherein:

the dashed line represents an S-configuration or an R-configuration at position 6′;

OP is an acceptor protected hydroxyl group;

AP is an acceptor protected amine group;

R₁ is as defined herein for Formula I or Ia;

A is an acceptor protected hydroxyl group (OP); or can otherwise be one of the other groups defining OR₂, either protected or unprotected, according to the chemical nature of these groups and the reaction conditions; and

B is a protected or unprotected form of the groups defining R₇.

According to some embodiments of the present invention, the acceptor hydroxyl-protected group is O-acetyl.

According to some embodiments of the present invention, the acceptor hydroxyl-protected group is O-TMS.

Other hydroxy-protecting groups are also contemplated.

According to some embodiments of the present invention, the acceptor amino-protecting group is azido, although other protecting groups are contemplated.

The acceptor hydroxyl-protected groups and the acceptor amino-protected groups at the various positions of the acceptors can be the same or different at each position.

In some embodiments, the acceptor is prepared by generating the moiety B, prior to reacting it with the donor.

The structure of the acceptor compound sets the absolute structure of Ring I and Ring II in the resulting compound according to some embodiments of the present invention.

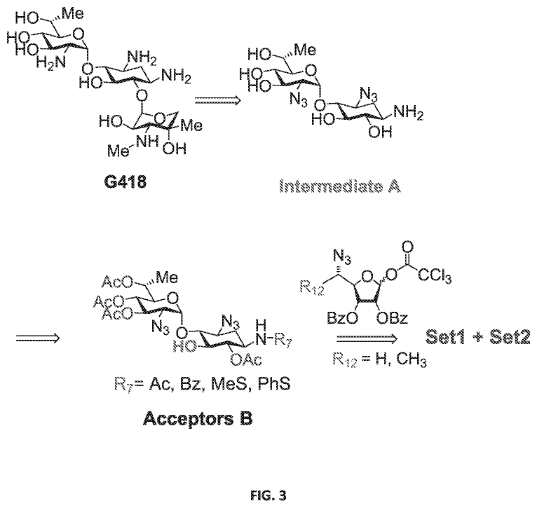

Exemplary acceptors suitable for use in preparing compounds of Formulae III or Ma are presented in the Examples section that follows and accompanying FIGS. 3-6 and 15-20.

In some embodiments, the synthesis of pseudo-disaccharide compounds of Formula Ia, according to some embodiments of the present invention, is accomplished using an amino-protected compound of Formula VI:

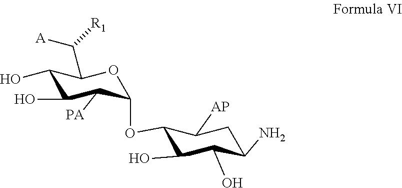

wherein:

the dashed line represents an S-configuration or an R-configuration at position 6′;

AP is an acceptor protected amine group;

R₁ is as defined herein for Formula Ia;

A is an acceptor protected hydroxyl group (OP), as described herein; or can otherwise be one of the other groups defining OR₂, either protected or unprotected, according to the chemical nature of these groups and the reaction conditions.

The compound of Formula IV is converted to the groups defining R₇ in Formula Ia, using methods known in the art.

Exemplary amino-protected compounds suitable for use in preparing compounds of Formulae I or Ia are presented in the Examples section that follows and accompanying Figures (see, for example, FIGS. 3-5).

Exemplary acceptor molecules suitable for use in the preparation of the compounds described herein under Formula III*a, are represented by Formula VII:

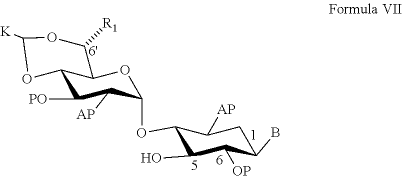

wherein:

the dashed line represents an S-configuration or an R-configuration at position 6′;

OP is an acceptor protected hydroxyl group;

AP is an acceptor protected amine group;

R₁ is as defined herein;

B is an acceptor protected amine group, in case R₇ is Formula Ia is hydrogen, or can otherwise be a protected or unprotected form of the groups defining R₇; and

K is a protected or unprotected form of the groups defining Rw.

According to some embodiments of the present invention, the acceptor hydroxyl-protected group is O-acetyl.

According to some embodiments of the present invention, the acceptor hydroxyl-protected group is O-TMS.

Other hydroxy-protecting groups are also contemplated.

According to some embodiments of the present invention, the acceptor amino-protecting group is azido, although other protecting groups are contemplated.

The acceptor hydroxyl-protected groups and the acceptor amino-protected groups at the various positions of the acceptors can be the same or different at each position.

In some embodiments, the acceptor is prepared by generating the moiety B, if applicable, prior to reacting it with the donor.

The structure of the acceptor compound sets the absolute structure of Ring I and Ring II in the resulting compound according to some embodiments of the present invention.

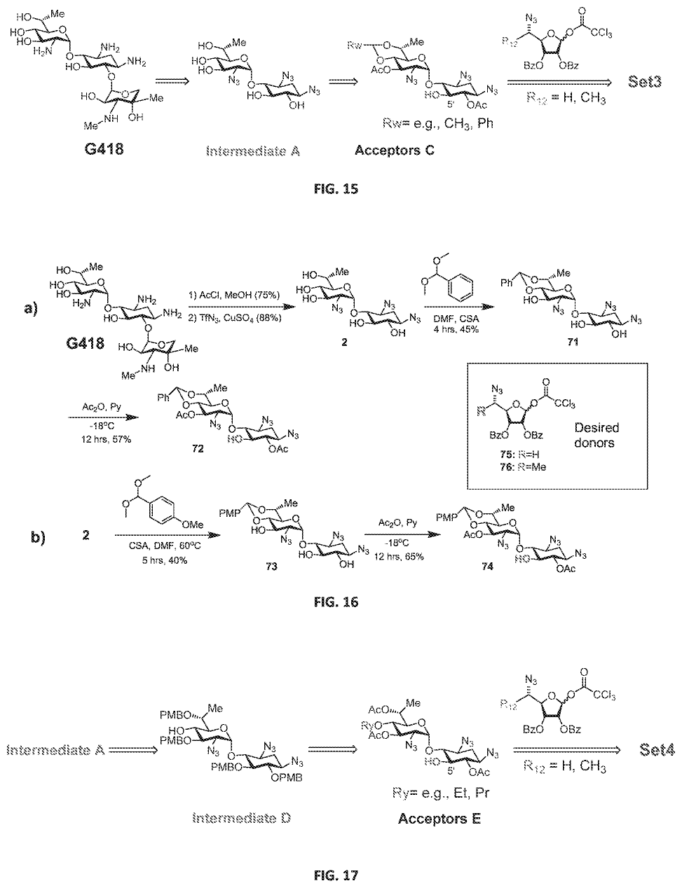

Exemplary acceptors suitable for use in preparing compounds of Formula III*a are presented in the Examples section that follows and accompanying Figures (see, for example, FIGS. 15 and 16).

Embodiments of the present invention further encompass any of the intermediate compounds described herein in the context of processes of preparing the compounds of the present embodiments.

Therapeutic Uses:

As known in the art, about a third of alleles causing genetic diseases carry premature termination (stop) codons (PTCs), which lead to the production of truncated proteins. One possible therapeutic approach involves the induction and/or promotion of readthrough of such PTCs to enable synthesis of full-length proteins. PTCs originate from either mutations, such as nonsense mutations, frame-shift deletions and insertions, or from aberrant splicing that generates mRNA isoforms with truncated reading frames. These mutations can lead to the production of truncated, nonfunctional or deleterious proteins, owing to dominant negative or gain-of-function effects.

In general, readthrough of PTCs can be achieved by suppressor transfer RNAs (tRNAs), factors that decrease translation-termination efficiency, such as small-interfering RNAs (siRNAs) directed against the translation-termination factors, and RNA antisense that targets the nonsense mutation region. One of the objectives of the present invention is to provide a pharmacological therapeutic approach aimed at achieving sufficient levels of functional proteins in a subject suffering from at least one genetic disorder associated with at least one premature stop-codon mutation.

According to some embodiments of the present invention, the provided therapeutic approach is aimed at inducing and/or promoting translational readthrough of the disease causing PTCs, to enable the synthesis and expression of full-length functional proteins.

As presented hereinabove, one extensively studied approach that has reached clinical trials, is based on readthrough by drugs affecting the ribosome decoding site, such as aminoglycoside antibiotics; however, aminoglycosides have severe adverse side effects when used at high concentrations and/or used long-term. The compounds presented herein were designed to exhibit an ability to induce and/or promote readthrough of a premature stop-codon mutation in an organism having such a mutation, while exhibiting minimal adverse effects. Such an activity renders these compounds suitable for use as therapeutically active agents for the treatment of genetic disorders associated with a premature stop-codon mutation.

As demonstrated in the Examples section that follows, exemplary compounds encompassed by the present embodiments were indeed shown to exhibit a premature stop-codon mutation suppression activity, and as useful in inducing readthrough of genes characterized by a disease-causing premature stop-codon mutation, and thus exhibit usefulness in treating respective genetic diseases or disorders associated with a premature stop-codon mutation.

Another, recently suggested, approach for suppressing stop-codon mutations (PTCs) involves attenuation of nonsense-mediated mRNA decay (NMD), a conserved

eukaryotic cellular pathway that targets PTC-containing mRNAs for degradation. It has been shown that attenuating NMD increases the abundance of PTC-containing mRNAs and thereby restores higher levels of the functional protein produced by PTC suppression [See, for example, Keeling et al., PLoS ONE 8(4): e60478, 2013; Bidou et. Al., RNA Biology 14(3): 378-388, 2017]. Additional studies have shown that aminoglycosides such as described in WO 2012/066546 (e.g., NB124) exhibit attenuation of NMD, indicating that aminoglycoside compounds of the present embodiments can also attenuate NMD [see, for example, Alroy et al., Abstracts/Molecular genetics and metabolism 2018, 123(2):518].

According to an aspect of some embodiments of the present invention, any of the compounds presented herein, e.g., having Formula A, B, I, I*, III, III*, IV or IVa, preferably of

Formula A, B, I, I*, III or III*, including any of the respective embodiments of the compounds and any combinations thereof (and including compounds represented by Formula Ia, I**, I*a, I*b, IIIa, III**, III*a and, III*b), are for use in attenuating nonsense-mediated mRNA decay (NMD), and/or are for use in the manufacture of a medicament for attenuating nonsense-mediated mRNA decay (NMD) and/or for treating a disease or disorder associated with irregulated nonsense-mediated mRNA decay (NMD) and/or a disease or disorder that is treatable by attenuating nonsense-mediated mRNA decay (NMD). In some embodiments, the disease or disorder is a genetic disease or disorder as described herein in any of the respective embodiments. In some embodiments, the disease or disorder is cancer as described herein in any of the respective embodiments.

According to an aspect of some embodiments of the present invention, any of the compounds presented herein, e.g., having Formula A, B, I, I*, III, III*, IV or IVa, preferably of Formula A, B, I, I*, III or III*, including any of the respective embodiments of the compounds and any combinations thereof (and including compounds represented by Formula Ia, I**, I*a, I*b, IIIa, III**, III*a and, III*b), are for use in treating cancer, as defined herein, of for use in the manufacture of a medicament for treating cancer, as defined herein, or for use in a method of treating cancer, as defined herein. According to some embodiments, a compound as described herein is for use in inducing and/or promoting readthrough of a premature stop codon (nonsense) mutation in a tumor suppressing gene (e.g., p53). According to some embodiments, a compound as described herein is for use in treating cancer by attenuating NMD.

According to an aspect of some embodiments of the present invention, any of the compounds presented herein, e.g., having Formula A, B, I, I*, III, III*, IV or IVa, preferably of Formula A, B, I, I*, III or III*, including any of the respective embodiments of the compounds and any combinations thereof (and including compounds represented by Formula Ia, I**, I*a, I*b, IIIa, III**, III*a and, III*b), are for use in inducing and/or promoting readthrough of a premature stop codon mutation and/or for increasing an expression of a gene having a premature stop codon mutation, and/or are for use in the manufacture of a medicament for inducing and/or promoting readthrough of a premature stop codon mutation and/or for increasing an expression of a gene having a premature stop codon mutation.

According to an aspect of some embodiments of the present invention, any of the compounds presented herein e.g., having Formula A, B, I, I*, III, III*, IV or IVa, preferably of Formula A, B, I, I*, III or III*, including any of the respective embodiments of the compounds and any combinations thereof (and including compounds represented by Formula Ia, I**, I*a, I*b, IIIa, III**, III*a and, III*b), are for use in the treatment of a genetic disorder associated with a premature stop-codon mutation, or for use in the manufacture of a medicament for the treatment of a genetic disorder associated with a premature stop-codon mutation.

Any of the premature stop-codon mutations are contemplated. In some embodiments, the mutations are those having an RNA code of UGA, UAG or UAA.

According to some of any of the embodiments described herein, the protein is translated in a cytoplasmic translation system.

According to some of any of the embodiments described herein, the compound is used in a mutation suppression amount.

According to some of any of the embodiments described herein, an inhibition of translation IC₅₀ of the compound in a eukaryotic cytoplasmic translation system is greater that an inhibition of translation IC₅₀ of the compound in a ribosomal translation system.

According to some of any of the embodiments described herein, an inhibition of translation IC₅₀ of the compound in a eukaryotic cytoplasmic translation system is greater that an inhibition of translation IC₅₀ of the compound in a prokaryotic translation system.

According to an aspect of some embodiments of the present invention, any of the compounds presented herein e.g., having Formula A, B, I, I*, III, III*, IV or IVa, preferably of Formula A, B, I, I*, III or III*, including any of the respective embodiments of the compounds and any combinations thereof (and including compounds represented by Formula Ia, I**, I*a, I*b, IIIa, III**, III*a and, III*b), are for use in the treatment of a genetic disorder associated with a premature stop-codon mutation, or for use in the manufacture of a medicament for the treatment of a genetic disorder associated with a premature stop-codon mutation.

According to an aspect of some embodiments of the present invention there is provided a method of treating a genetic disorder associated with a premature stop-codon mutation. The method, according to this aspect of the present invention, is effected by administering to a subject in need thereof a therapeutically effective amount of one or more of the compounds presented herein, e.g., having Formula A, B, I, I*, III, III*, IV or IVa, preferably of Formula A, B, I, I*, III or III*, including any of the respective embodiments of the compounds and any combinations thereof (and including compounds represented by Formula Ia, I**, I*a, I*b, IIIa, III**, III*a and III*b).

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the phrase “therapeutically effective amount” describes an amount of the polymer being administered which will relieve to some extent one or more of the symptoms of the condition being treated.

The phrase “genetic disorder”, as used herein, refers to a chronic disorder which is caused by one or more defective genes that are often inherited from the parents, and which can occur unexpectedly when two healthy carriers of a defective recessive gene reproduce, or when the defective gene is dominant. Genetic disorders can occur in different inheritance patterns which include the autosomal dominant pattern wherein only one mutated copy of the gene is needed for an offspring to be affected, and the autosomal recessive pattern wherein two copies of the gene must be mutated for an offspring to be affected.

The phrase “genetic disorder”, as used herein, encompasses a genetic disorder, genetic disease, genetic condition or genetic syndrome.

According to some of any of the embodiments of the present invention, the genetic disorder, genetic disease, genetic condition or genetic syndrome, involves a gene having a premature stop-codon mutation, also referred to herein as a truncation mutation and/or a nonsense mutation, which leads to improper translation thereof. The improper translation produces a dysfunctional essential protein or causes a reduction or abolishment of synthesis of an essential protein. In the context of the some embodiments of the present invention, the genetic disorders which are contemplated within the scope of the present embodiments are referred to as genetic disorders associated with a premature stop-codon mutation and/or a protein truncation phenotype.

According to some of any of the embodiments of the present invention, a genetic disorder associated with a premature stop-codon mutation and/or a protein truncation phenotype is treatable by inducing and/or promoting readthrough of the mutation in the complete but otherwise defective transcript (mRNA), or in other words, by inducing and/or promoting suppression of the nonsense mutation (the premature stop-codon mutation and/or the truncation mutation). In the context of embodiments of the present invention, a genetic disorder is one that is treatable by readthrough-inducing and/or promoting compounds.

Methods for identification of a genetic disorder associated with a premature stop-codon mutation and/or a protein truncation phenotype are well known in the art, and include full or partial genome elucidation, genetic biomarker detection, phenotype classification and hereditary information analysis.

Such methods often result in pairs of mutant/wild type (WT) sequences, and these pairs can be used in known methodologies for identifying if the genetic disorder is associated with a premature stop-codon mutation and/or a protein truncation phenotype.

A readthrough-inducing/promoting activity of compounds for treating such genetic disorders can be established by methods well known in the art.

For example, a plasmid comprising two reporter genes interrupted by a sequence of the mutated gene (the genetic disorder-causing gene) is transected into a protein expression platform, either in full cells or in a cell-free systems, and the ratio between the expression level of the two genes in the presence of a tested compound is measured, typically in series of concentrations and duplications, and compared to the gene expression level ratio of the wild-type and/or to the expression level ratio measured in a control sample not containing the tested compound.

It is noted that the experimental model for readthrough activity, namely the nucleotide sequence of gene containing the premature stop-codon mutation, is a byproduct of the process of identifying a genetic disorder as associated with a premature stop-codon mutation and/or a protein truncation phenotype, and further noted that with the great advances in genomic data acquisition, this process is now well within the skills of the artisans of the art, and that once the mechanism of action of a drug candidate is established, as in the case of genetic disorders which have been shown to be associated with a premature stop-codon mutation and/or a protein truncation phenotype, it is well within the skills of the artisans of the art to identify, characterize and assess the efficacy, selectivity and safety of any one of the readthrough-inducing compounds presented herein. It is further well within the skills of the artisans of the art to take the readthrough-inducing compounds presented herein further through the routine processes of drug development.

Methodologies for testing readthrough of a premature stop-codon mutation and/or a truncation mutation, referred to herein as readthrough activity, are known in the art, and several exemplary experimental methods are provided in the Examples section that follows, by which the readthrough-inducing compounds, according to some embodiments of the present invention, can be characterized. It is to be understood that other methods can be used to characterized readthrough-inducing compounds, and such methods are also contemplated within the scope of the present invention. Methods such as provided herein can also be adapted for high throughput screening technology that can assay thousands of compounds in a relatively short period of time.

The skilled artisan would appreciate that many in vitro methodologies can be used to characterize readthrough-inducing compounds provided herein in terms of safety of use as drugs, and assess the drug candidates in terms of their cytotoxicity versus their efficacy. The skilled artisan would also appreciate that many in vitro methodologies can be used to characterize the readthrough-inducing compounds provided herein for eukaryotic versus prokaryotic selectivity, and such methodologies may also be adapted for high throughput screening technology that can assay thousands of compounds in a relatively short period of time.

Non-limiting examples of genetic disorders, diseases, conditions and syndromes, which are associated with the presence of at least one premature stop-codon or other nonsense mutations include cancer, Rett syndrome, cystic fibrosis (CF), Becker's muscular dystrophy (BMD), Congenital muscular dystrophy (CMD), Duchenne muscular dystrophy (DMD), Factor VII deficiency, Familial atrial fibrillation, Hailey-Hailey disease, hemophilia A, hemophilia B, Hurler syndrome, Louis—Bar syndrome (ataxia-telangiectasia, AT), McArdle disease, Mucopolysaccharidosis, Nephropathic cystinosis, Polycystic kidney disease, type I, II and III Spinal muscular atrophy (SMA), Tay-Sachs, Usher syndrome, cystinosis, Severe epidermolysis bullosa, Dravet syndrome, X-linked nephrogenic diabetes insipidus (XNDI) and X-linked retinitis pigmentosa.

Additional genetic disorders, diseases, conditions and syndromes, which are associated with the presence of at least one premature stop-codon or other nonsense mutations, are listed in “Suppression of nonsense mutations as a therapeutic approach to treat genetic diseases” by Kim M. Keeling, K. M Bedwell, D. M., Wiley Interdisciplinary Reviews: RNA, 2011, 2(6), p. 837-852; “Cancer syndromes and therapy by stop-codon readthrough”, by Bordeira-Carrico, R. et al., Trends in Molecular Medicine, 2012, 18(11), p. 667-678, and any references cited therein, all of which are incorporated herewith by reference in their entirety.

As used herein, the terms “cancer”, “cancerous disease” and “tumor” are interchangeably used. The terms refer to a malignant growth and/or tumor caused by abnormal and uncontrolled cell proliferation (cell division). The term “cancer” encompasses tumor metastases. The term “cancer cell” describes the cells forming the malignant growth or tumor.

Non-limiting examples of cancers and/or tumor metastases include any solid or non-solid cancer and/or tumor metastasis, including, but not limiting to, tumors of the gastrointestinal tract (e.g., colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic breast cancer, susceptibility to breast cancer, type 4 breast cancer, breast cancer-1, breast cancer-3, breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B-cell lymphoma, Diffuse large B-cell lymphoma (DLBCL), Burkitt lymphoma, cutaneous T-cell lymphoma, histiocytic lymphoma, lymphoblastic lymphoma, T-cell lymphoma, thymic lymphoma), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, hi stiocytoma, hybridoma (e.g., B-cell), hypernephroma, insulinoma, islet tumor, keratoma, leiomyoblastoma, leiomyosarcoma, leukemia (e.g., acute lymphatic leukemia, acute lymphoblastic leukemia, acute lymphoblastic pre-B cell leukemia, acute lymphoblastic T cell leukemia, acute megakaryoblastic leukemia, monocytic leukemia, acute myelogenous leukemia, acute myeloid leukemia, acute myeloid leukemia with eosinophilia, B-cell leukemia, basophilic leukemia, chronic myeloid leukemia, chronic B-cell leukemia, eosinophilic leukemia, Friend leukemia, granulocytic or myelocytic leukemia, hairy cell leukemia, lymphocytic leukemia, megakaryoblastic leukemia, monocytic leukemia, monocytic-macrophage leukemia, myeloblastic leukemia, myeloid leukemia, myelomonocytic leukemia, plasma cell leukemia, pre-B cell leukemia, promyelocytic leukemia, subacute leukemia, T-cell leukemia, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme, multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

In any of the methods and uses described herein, the compounds described herein can be utilized either per se or form a part of a pharmaceutical composition, which further comprises a pharmaceutically acceptable carrier, as defined herein.

According to an aspect of some embodiments of the present invention, there is provided a pharmaceutical composition which comprises, as an active ingredient, any of the novel compounds described herein and a pharmaceutically acceptable carrier.

As used herein a “pharmaceutical composition” refers to a preparation of the compounds presented herein, with other chemical components such as pharmaceutically acceptable and suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, Pa., latest edition, which is incorporated herein by reference.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the compounds presented herein into preparations which, can be used pharmaceutically. Proper formulation is dependent upon the route of administration chosen.

According to some embodiments, the administration is effected orally. For oral administration, the compounds presented herein can be formulated readily by combining the compounds with pharmaceutically acceptable carriers well known in the art. Such carriers enable the compounds presented herein to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the compounds presented herein may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For injection, the compounds presented herein may be formulated in aqueous solutions, preferably in physiologically compatible buffers such as Hank's solution, Ringer's solution, or physiological saline buffer with or without organic solvents such as propylene glycol, polyethylene glycol.

For transmucosal administration, penetrants are used in the formulation. Such penetrants are generally known in the art.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active aminoglycoside compounds doses.

For buccal administration, the compositions may take the form of tablets or lozenges formulated in conventional manner.

For administration by inhalation, the compounds presented herein are conveniently delivered in the form of an aerosol spray presentation (which typically includes powdered, liquefied and/or gaseous carriers) from a pressurized pack or a nebulizer, with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compounds presented herein and a suitable powder base such as, but not limited to, lactose or starch.

The compounds presented herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the compounds preparation in water-soluble form. Additionally, suspensions of the compounds presented herein may be prepared as appropriate oily injection suspensions and emulsions (e.g., water-in-oil, oil-in-water or water-in-oil in oil emulsions). Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the compounds presented herein to allow for the preparation of highly concentrated solutions.

Alternatively, the compounds presented herein may be in powder form for constitution with a suitable vehicle, e.g., sterile, pyrogen-free water, before use.

The compounds presented herein may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of compounds presented herein effective to prevent, alleviate or ameliorate symptoms of the disorder, or prolong the survival of the subject being treated.

Determination of a therapeutically effective amount is well within the capability of those skilled in the art, especially in light of the detailed disclosure provided herein.

For any compounds presented herein used in the methods of the present embodiments, the therapeutically effective amount or dose can be estimated initially from activity assays in animals. For example, a dose can be formulated in animal models to achieve a circulating concentration range that includes the mutation suppression levels as determined by activity assays (e.g., the concentration of the test compounds which achieves a substantial read-through of the truncation mutation). Such information can be used to more accurately determine useful doses in humans.

Toxicity and therapeutic efficacy of the compounds presented herein can be determined by standard pharmaceutical procedures in experimental animals, e.g., by determining the EC₅₀ (the concentration of a compound where 50% of its maximal effect is observed) and the LD₅₀ (lethal dose causing death in 50% of the tested animals) for a subject compound. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p.1).

Dosage amount and interval may be adjusted individually to provide plasma levels of the compounds presented herein which are sufficient to maintain the desired effects, termed the minimal effective concentration (MEC). The MEC will vary for each preparation, but can be estimated from in vitro data; e.g., the concentration of the compounds necessary to achieve 50-90% expression of the whole gene having a truncation mutation, i.e. read-through of the mutation codon. Dosages necessary to achieve the MEC will depend on individual characteristics and route of administration. HPLC assays or bioassays can be used to determine plasma concentrations.

Dosage intervals can also be determined using the MEC value. Preparations should be administered using a regimen, which maintains plasma levels above the MEC for 10-90% of the time, preferable between 30-90% and most preferably 50-90%.

Depending on the severity and responsiveness of the chronic condition to be treated, dosing can also be a single periodic administration of a slow release composition described hereinabove, with course of periodic treatment lasting from several days to several weeks or until sufficient amelioration is effected during the periodic treatment or substantial diminution of the disorder state is achieved for the periodic treatment.

The amount of a composition to be administered will, of course, be dependent on the subject being treated, the severity of the affliction, the manner of administration, the judgment of the prescribing physician, etc.. Compositions of the present invention may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a compound according to the present embodiments, formulated in a compatible pharmaceutical carrier may also be prepared, placed in an appropriate container, and labeled for treatment of an indicated condition or diagnosis, as is detailed hereinabove.

Thus, in some embodiments, the pharmaceutical composition is packaged in a packaging material and identified in print, in or on the packaging material, for use in the treatment of a genetic disorder, as defined herein, and/or in any of the uses described herein.

In some embodiments, the pharmaceutical composition is for use in the treatment of a genetic disorder, as defined herein, and/or in any of the uses described herein.

In any of the composition, methods and uses described herein, the compounds can be utilized in combination with other agents useful in the treatment of the genetic disorder and/or in inducing or promoting readthrough activity of a premature stop codon mutation and/or in increasing expression of a gene having a premature stop codon mutation as described herein.

Exemplary such agents include, but are not limited to, CFTR potentiators such as, for example, ivacaftor (VX-770) (see, X. Xue et al., Am. J. Respir. Cell Mol. Biol. 50 (4), 805-816 (2014); and agents that attenuate Nonsense-Mediated mRNA Decay (NMD), such as for example, NMDI-1, caffeine and other agents that disrupt the UPF1 phosphorylation cycle (see, K. M. Keeling et al., PLoS ONE 8 (4), e60478 (2013)). Any other agents are contemplated.

Being primarily directed at treating genetic disorders, which are chronic by definition, the compounds presented herein or pharmaceutical compositions containing the same are expected to be administered throughout the lifetime of the subject being treated. Therefore, the mode of administration of pharmaceutical compositions containing the compounds should be such that will be easy and comfortable for administration, preferably by self-administration, and such that will take the smallest toll on the patient's wellbeing and course of life.

The repetitive and periodic administration of the compounds presented herein or the pharmaceutical compositions containing the same can be effected, for example, on a daily basis, i.e. once a day, more preferably the administration is effected on a weekly basis, i.e. once a week, more preferably the administration is effected on a monthly basis, i.e. once a month, and most preferably the administration is effected once every several months (e.g., every 1.5 months, 2 months, 3 months, 4 months, 5 months, or even 6 months).

As discussed hereinabove, some of the limitations for using presently known aminoglycosides as truncation mutation readthrough drugs are associated with the fact that they are primarily antibacterial (used as antibiotic agents). Chronic use of any antibacterial agents is highly unwarranted and even life threatening as it alters intestinal microbial flora which may cause or worsen other medical conditions such as flaring of inflammatory bowel disease, and may cause the emergence of resistance in some pathological strains of microorganisms.

In some embodiments, the compounds presented herein have substantially no antibacterial activity. By “no antibacterial activity” it is meant that the minimal inhibition concentration (MIC) thereof for a particular strain is much higher than the concentration of a compound that is considered an antibiotic with respect to this strain. Further, the MIC of these compounds is notably higher than the concentration required for exerting truncation mutation suppression activity.

Being substantially non-bactericidal, the compounds presented herein do not exert the aforementioned adverse effects and hence can be administered via absorption paths that may contain benign and/or beneficial microorganisms that are not targeted and thus their preservation may even be required. This important characteristic of the compounds presented herein renders these compounds particularly effective drugs against chronic conditions since they can be administered repetitively and during life time, without causing any antibacterial-related adverse, accumulating effects, and can further be administered orally or rectally, i.e. via the GI tract, which is a very helpful and important characteristic for a drug directed at treating chronic disorders.

According to some embodiments, the compounds presented herein are selected and/or designed to be selective towards the eukaryotic cellular translation system versus that of prokaryotic cells, namely the compounds exhibit higher activity in eukaryotic cells, such as those of mammalian (humans) as compared to their activity in prokaryotic cells, such as those of bacteria. Without being bound by any particular theory, it is assumed that the compounds presented herein, which are known to act by binding to the A-site of the 16S ribosomal RNA while the ribosome is involved in translating a gene, have a higher affinity to the eukaryotic ribosomal A-site, or otherwise are selective towards the eukaryotic A-site, versus the prokaryotic ribosomal A-site, as well as the mitochondrial ribosomal A-site which resembles its prokaryotic counterpart.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is expected that during the life of a patent maturing from this application many relevant genetic diseases and disorders as defined herein will be uncovered and the scope of this term is intended to include all such new disorders and diseases a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES Reference is now made to the following examples, which together with the above descriptions illustrate some embodiments of the invention in a non limiting fashion.

### Materials and Methods

¹H-NMR, ¹³C-NMR, DEPT, COSY, 1D TOCSY, HMQC, and HMBC spectra were recorded on a Bruker Avance™ 500/400 (excluding 1D TOCSY)/200 (only ¹H NMR) spectrometers. Chemical shifts reported (in ppm) are relative to internal Me₄Si (δ=0.0) with CDCl₃ as the solvent, and to HOD (δ=4.63) with D₂O as the solvent, unless otherwise indicated.

Mass spectral analyses were performed on a Bruker Daltonix Apex 3 mass spectrometer under electron spray ionization (ESI), TSQ-70B mass spectrometer (Finnigan Mat) or under MALDI-TOF on a a-cyano-4-hydroxycinnamic acid matrix on a MALDI Micromass spectrometer.

Reactions were monitored by TLC on Silica Gel 60 F254 (0.25 mm, Merck), and spots were visualized by charring with a yellow solution containing (NH₄)₆Mo₇)₂₄.4H₂O (120gr) and (NH₄)₂Ce(NO₃)₆ (5gr) in 10% H₂SO₄ (800 mL).

Flash column chromatography was performed on Silica gel Gel 60 (70-230 mesh).

All reactions were carried out with anhydrous solvents, unless otherwise noted.

All chemicals, unless otherwise stated, were obtained from commercial sources.

### Example 1

### Chemical Syntheses of Exemplary N1-Substituted Compounds According to Some of the Present Embodiments

A library of newly designed aminoglycoside derivatives featuring acyl and sulfinyl groups as substituents at the N1 position was prepared. The initial library was based on the modification of the previously described lead candidate NB74 (see, Table 1) with acetate (NB74-N1Ac), benzoate (NB74-N1Bz), methylsulfonate (NB74-N1MeS) and phenylsulfonate (NB74-N1PhS) at N1 position (see, FIG. 2, Sett). Next, similar modifications of the previously described lead candidate NB124 (see, Table 1) at the N1 position were performed to afford compounds NB124-N1Ac, NB124-N1Bz and NB124-N1MeS (see, FIG. 2, Set2).

The exemplary newly designed compounds were prepared according to the general synthetic pathway presented in FIG. 3.

Briefly, the commercially available G418 was first converted to the known common intermediate A in four chemical steps according to the previously reported synthetic steps (see, Nudelman et al., Bioorg. Med. Chem. 18, 3735-46 (2010)). The intermediate A was then acetylated or sulfonated at the free amine (N1 position) followed by the selective acetylation to afford the series of common acceptors B in which N1 is modified with different amide or sulfonamide moieties and the free hydroxyl at C-5 position ready for the coupling of the ring III, if desired. Coupling steps of Ring III are performed according to previously reported synthetic steps, using the respective trichloroacetimidate donors, followed by a two-step deprotection to afford the desired Set1 and Set2 structures.

The following are processes of preparing exemplary compounds according to some embodiments of the present invention, which are presented in Table 1 hereinabove and in FIG. 2.

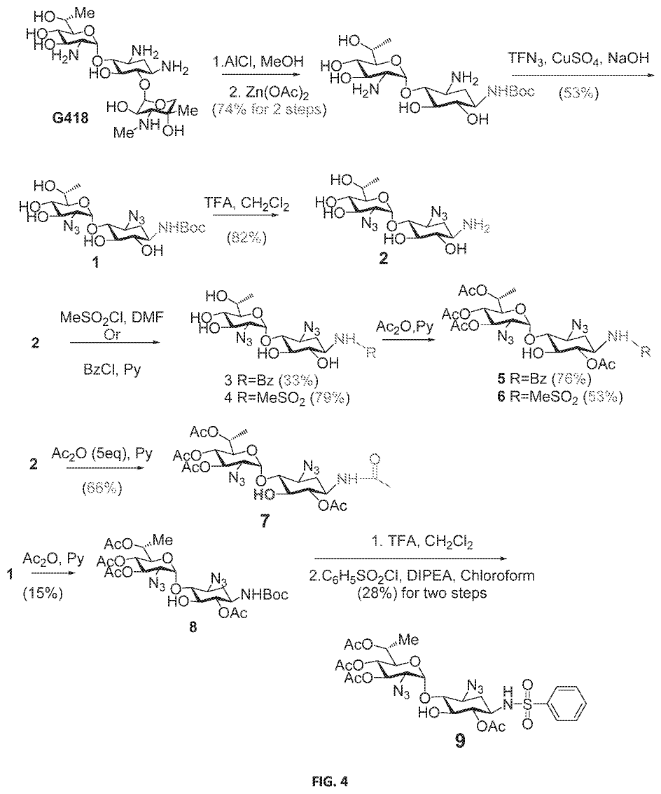

FIG. 4 presents the synthetic pathway for converting G418 into Acceptor Compounds 5, 6,7and9.

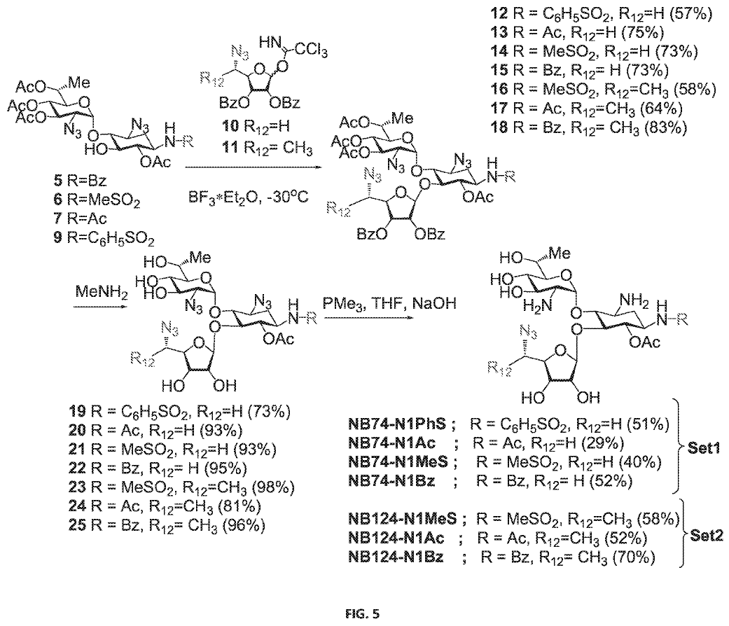

FIG. 5 presents the synthetic pathway for converting acceptors 5-7 and 9 into the respective N1-modified NB-74 and NB-124.

### Preparation of 2′,3-Diazido-1-N-benzamide-6′-(R)-methyl-paromamine (Compound 3; FIG. 4)

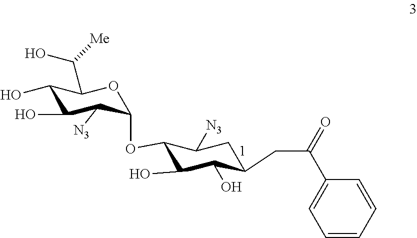

Compound 2, prepared as described in Nudelman, I. et al. Bioorganic Med. Chem. 18, 3735-3746 (2010) (2 grams, 5.13 mmol), was dissolved in dry Pyridine (10 ml). The stirring mixture was cooled on ice bath to 0° C., followed by addition of benzoyl chloride (12 equivalents, 0.061 mol, 7 ml). Propagation of the reaction was monitored by TLC (EtOAc/Hexane 3:7). The mixture was diluted with EtOAc and washed with HCl 1M, NaHCO₃ (Saturated) and brine. The combined organic layer was dried over MgSO₄, the mixture was evaporated to dryness and treated with MeNH₂ (33% solution in EtOH). The propagation of the reaction was monitored by TLC (EtOAc/MeOH, 1:1) which indicated completion after 12 hours. The reaction mixture was evaporated to dryness and purified by silica gel chromatography (100% EtOAc) to yield Compound 3 (2 grams, 79%).

¹HNMR (500 MHz, MeOD): Ring I: δ=5.77 (d,1H, J=3.5 Hz, H-1), 4.06 (dd, 1H,J=6.8, 3.6 Hz, H-6), 4.02-3.93 (m, 2H, H-3,H-5), 3.40 (dd,1H, J=10.0, 8.7 Hz, H-4), 3.11 (dd, 1H,J=10.6, 4.2 Hz, H-2), 1.28 (d, 3H,J=6.1 Hz, CH₃—C-6). Ring II: δ=4.04 (ddd,1H, J=11.9, 10.1, 4.4 Hz, H-1), 3.66-3.54 (m,3H, H-3, H-6, H-5), 3.45 (dd,1H, J=10.1, 9.0 Hz, H-4), 2.34-2.29 (m, 1H, H-2), 1.58 (dd, 1H, J=25.1, 12.3 Hz, H-2). Additional peaks in the spectrum were identified as follow: δ=7.85 (d, J=7.2 Hz, 2H, Ph), 7.53 (t, J=7.4 Hz, 1H, Ph), 7.45 (t, J=7.6 Hz, 2H, Ph).

¹³C NMR (126 MHz, MeOD): δ=169.02 (Carbonyl), 134.28 (Ph), 131.24 (Ph), 128.04 (Ph), 126.98 (Ph), 97.31 (C-1′), 78.93 (C-4), 77.31 (C-5), 74.59 (C-6), 73.76 (C-3′), 72.83 (C-4′), 70.91 (C-5′), 67.91 (C-1), 63.26 (C-2′), 60.11 (C-3), 49.70 (C-6′), 32.45 (C-2), 16.58 (CH₃—C-6′).

MALDI TOF MS: C₂₀H₂₇N₇O₈ ([M+H]⁺) m/e 493.47; measured m/e 494.2.

### Preparation of 2′,3-Diazido-1-N-methanesulfonamide-6′-(R)-methyl-paromamine (Compound 4; FIG. 4)

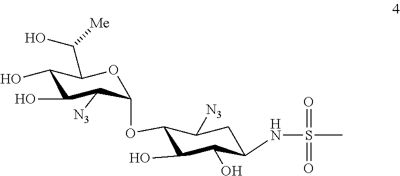

Compound 2, prepared as described in Nudelman, I. et al. 2010 (supra) (2.5 grams, 6.42 mmol) was dissolved in DMF followed by addition of methanesulfonyl chloride (1 equivalent, 0.5 ml) and the obtained mixture was stirred at room temperature for 24 hours. Propagation of the reaction was monitored by TLC (MeOH/EtOAc 1:9). The mixture was thereafter evaporated under vacuum and purified by silica gel chromatography. The product was eluted at 100% EtOAc. Fractions containing the product were combined and evaporated under vacuum to afford Compound 4 (1 gram, 33%).

¹H NMR (500 MHz, MeOD): Ring I: δ=5.70 (d,1H, J=3.5 Hz, H-1), 4.04-4.00 (m, 1H, H-6), 3.96-3.88 (m, 2H, H-3,H-5), 3.35 (t,1H, J=9.5 Hz, H-4), 3.07 (dd,1H, J=10.4, 4.0 Hz, H-2), 1.24 (d, 1H, J=3.4 Hz, CH₃—C-6). Ring II: δ=4.88-4.87 (m, 1H, H-6), δ 3.54-3.44 (m, 2H, H-1, H-4), 3.24 (dd,1H, J=13.4, 6.6 Hz, H-3), 3.15 (t, 1H, J=7.7 Hz, H-5), 2.34-2.24 (m, 1H, H-2 eq), 1.51-1.43 (m, 1H, H-2 ax). Additional peaks in the spectrum were identified as follow: δ=3.01 (s, 3H, NHSO₂—CH₃).

¹³C NMR (126 MHz, MeOD): δ=95.73 (C-1′), 77.24 (C-4), 75.50 (C-1), 73.70 (C-5), 72.24 (C-5′), 71.30 (C-4′), 69.43 (C-3′), 66.37 (C-6′), 61.74 (C-2′), 58.29 (C-6), 51.86 (C-3), 38.70 (NHSO₂—CH₃), 33.14 (C-2), 15.06 (CH₃—C-6′).

MALDI TOFMS: C₁₄H₂₅N₇O₉S ([M+H]⁺) m/e 467.47; measured m/e 668.29.

### Preparation of 3′,4′,6′,6-Tetra-O-acetate-2′,3-Diazido-1-N-benzamide-6′-(R)-methyl-paromamine (Compound 5; FIG. 4):

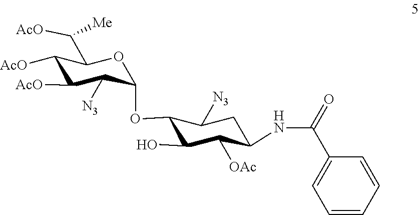

Compound 3 (0.380 gram, 0.770 mmol) was dissolved in dry pyridine (5 ml), the solution was cooled at −18° C. and then acetic anhydride (4.5 equivalent, 0.3 ml, 3 mmol) was added. The reaction temperature was kept −18° C. and the reaction progress was monitored by TLC (EtOAc/Hexane 7:3), which indicated completion after 12 hours. The reaction mixture was diluted with EtOAc (15 ml) and extracted with an aqueous solution of HCl 1N, saturated aqueous solution of NaHCO₃, and brine. The combined organic layer was dried over MgSO₄ and concentrated. The crude product was purified by silica gel chromatography (EtOAc/Hexane 1:1) to afford Compound 5 (0.387 gram, 76% yield).

¹HNMR (500 MHz, CDCl₃): Ring I: δ=5.55-5.49 (m, 1H, H-3), 5.37 (d, 1H, J=3.5 Hz, 4H, H-1), 5.02-4.96 (m, 2H, H-4,H-6), 4.35 (dd, 1H,J=10.4, 1.8 Hz, H-5), 3.66-3.62 (m, 1H, H-2), 1.26 (d, 3H, J=6.0 Hz, CH₃—C-6). Ring II: δ=6.66 (d,1H, J=7.4 Hz, amide), 4.92-4.87 (m, 1H, H-6), 4.27-4.20 (m, 1H,H-1), 3.84 (t,1H, J=9.2 Hz, H-5), 3.55-3.47 (m, 1H, H-3), 3.46-3.39 (m, 1H, H-4), 2.67 (dt, 1H, J=12.4, 4.2 Hz, H-2 eq), 1.55-1.47 (m, 1H, H-2 ax). Additional peaks in the spectrum were identified as follow: δ=7.73-7.70 (m, 2H, Ph), 7.53 (dd, 1H, J=4.8, 3.7 Hz, Ph), 7.43 (t, 2H, J=7.6 Hz, Ph), 2.11 (s, 3H, Ac), 2.10 (s, 3H, Ac), 2.09 (s, 3H, Ac), 2.06 (s, 3H, Ac).

¹³C NMR (126 MHz, CDCl₃): δ=172.61 (Carbonyl), 170.12 (Carbonyl), 169.95 (Carbonyl), 169.94 (Carbonyl), 167.06 (Amide), 133.37 (Ph), 131.91 (Ph), 128.68 (Ph), 126.86 (Ph), 98.64 (C-1′), 83.50 (C-4), 74.67 (C-6), 74.07 (C-5), 71.34 (C-3′), 70.90 (C-5′), 69.08 (C-4′), 68.85 (C-6′), 61.64 (C-2′), 58.41 (C-3), 48.65 (C-1), 32.89 (C-2), 21.01 (Ac), 20.91 (Ac), 20.69 (Ac), 20.62 (Ac), 14.01 (CH₃—C-6′).

MALDI TOFMS: C₂₃H₃₃N₇O₁₂ ([M+H]⁺) m/e 661.1; measured m/e 622.1.

### Preparation of 3′,4′,6′,6-Tetra-O-acetate-2′,3-Diazido-1-N-methane sulfonamide-6′-(R)-methyl-paromamine (Compound 6; FIG. 4)

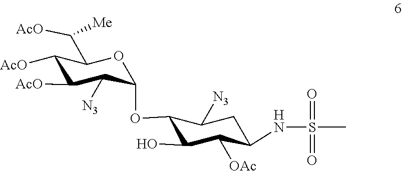

Compound 6 was prepared as described for the preparation of Compound 5 using Compound 4 (0.8 gram, 1.711 mmol) as the starting material and the pyridine (10 ml) and acetic anhydride (5 equivalents, 0.8 ml, 9 mmol), affording 0.576 gram (53%).

¹H NMR (500 MHz, MeOD): Ring I: δ=5.89 (d,1H, J=3.6 Hz, H-1), 5.49-5.44 (m, 1H,H-3), 4.99 (dd,2H, J=10.5, 9.0 Hz, H-4,H-6), 4.44 (dd,1H, J=10.6, 2.2 Hz, H-5), 3.43-3.39 (m, 1H, H-2), 1.29 (d, 3H, J=5.9 Hz, CH₃—C-6). Ring II: δ=4.76 —4.71 (m, 1H, H-6), 3.77 (t,1H, J=9.5 Hz, H-5), 3.68 (ddd,1H, J=11.9, 10.0, 4.9 Hz, H-1), 3.57-3.48 (m, 2H, H-3, H-4), 2.39 (dt,1H, J=12.5, 4.5 Hz, H-2 eq), 1.60 (dd, 1H, J=25.7, 12.5 Hz, H-2 ax). Additional peaks in the spectrum were identified as follow: δ=2.97 (s, 3H, NHSO₂—CHs), 2.13 (s, 3H, Ac), 2.07 (s, 6H, Ac), 2.06 (s, 3H,Ac).

¹³C NMR (126 MHz, MeOD): δ=171.01 (Carbonyl), 170.62 (Carbonyl), 170.18 (Carbonyl), 170.10 (Carbonyl), 97.53 (C-1′), 78.80 (C-4), 75.69 (C-6), 74.53 (C-5), 70.05 (C-3′), 70.00 (C-5′), 69.34 (C-4′), 68.66 (C-6′), 60.54 (C-2′), 59.47 (C-1), 50.91 (C-3), 40.42 (NHSO₂—CH₃), 19.74 (C-2), 19.70 (Ac), 19.18 (Ac), 19.15 (Ac), 12.33 (CH₃—C-6′).

MALDI TOFMS: C₂₂H₃₃N₇O₁₃S ([M+Na]⁺) m/e 635.60; measured m/e 658.05.

### Preparation of 3′,4′,6′,6-Tetra-O-acetate-2′,3-diazido-1-N-acetamide-6′-(R)-methyl-paromamine (Compound 7; FIG. 4)

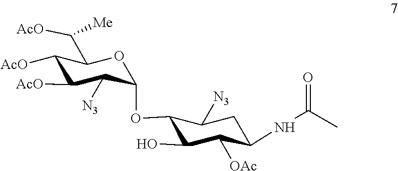

Compound 7 was prepared as described for the preparation of Compound 3 using Compound 2 as the starting material (0.1 gram, 0.526 mmol), pyridine (3 ml) and acetic anhydride (6.4 equivalents, 0.2 ml, 1.6 mmol), affording 0.1 gram (66%).

¹H NMR (500 MHz, MeOD): Ring I: δ=5.90 (d, 1H, J=3.5 Hz, H-1), 5.50-5.44 (m, 1H, H-3), 5.02-4.96 (m, 2H, H-4, H-6), 4.45 (dd, 1H, J=10.5, 1.7 Hz, H-5), 3.43-3.38 (m, 1H, H-2) 1.29 (d,3H, J=5.2 Hz, CH₃—C-6). Ring II: δ=4.76 (t, 1H, J=10.1 Hz, H-4), 4.09-4.00 (m, 1H, H-1), 3.80 (t, 1H, J=9.4 Hz, H-5), 3.71-3.62 (m, 1H,H-3), 3.55 (t, 1H, J=9.7 Hz, H-6), 2.24-2.17 (m, 1H, H-2 eq), 1.58 (q, 1H, J=12.7 Hz, H-2 ax). Additional peaks in the spectrum were identified as follow: δ=2.08 (s, 3H,Ac), 2.07 (s, 3H, Ac), 2.06 (s, 3H, Ac), 1.90 (s, 3H, Ac).

¹³CNMR (126MHz, MeOD): δ=171.43 (Amide), 171.07 (Carbonyl), 170.57 (Carbonyl), 170.16 (Carbonyl), 170.10 (Carbonyl), 97.55 (C-1′) 78.90 (C-3), 76.19 (C-6), 74.47 (C-1), 70.04 (C-3′), 70.00 (C-5′), 69.35 (C-4′), 68.67 (C-6′), 60.53 (C-2′), 59.68 (C-5), 32.23 (C-2), 21.21 (Ac), 19.73 (Ac), 19.39 (Ac), 19.18 (Ac), 19.16 (Ac), 12.35 (CH₃—C-6′);

MALDI TOFMS: C₂₃H₃₃N₇O₁₂ ([M+Na]⁺) m/e 599.55; measured m/e 622.08.

### Preparation of 3′,4′,6′,6-Tetra-O-acetate-2′,3-diazido-1-N-[(tert-butoxy) carbonyl]-6′-(R)-methyl-paromamine (Compound 8; FIG. 4)

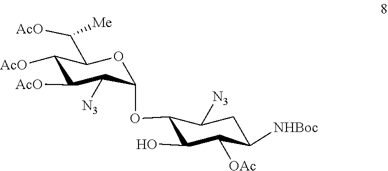

Compound 8 was prepared as described for the preparation of Compound 5 using Compound 1 as the starting material (0.3 gram, 0.610 mmol), pyridine (5 ml) and acetic anhydride (4.5 equivalents, 0.3 ml, 3 mmol), affording 0.060 gram (15%).

¹HNMR (400 MHz, CDCl₃): δ=5.56-5.45 (m, 1H), 5.35 (d, 1H, J=3.2 Hz, H-1′), 4.97 (dd, J=13.2, 6.4 Hz, 2H), 4.71 (dd, J=19.8, 9.5 Hz, 2H), 4.33 (dd, J=10.5, 1.6 Hz, 1H), 3.79-3.67 (m, 2H), 3.62 (dd, J=10.5, 3.4 Hz, 2H), 3.46-3.33 (m, 2H), 2.49-2.38 (m, 1H), 2.12 (s, 3H, Ac), 2.09 (s, 3H, Ac), 2.07 (s, 3H, Ac), 2.05 (s, 3H, Ac), 1.42 (s, 11H, Boc), 1.25 (d, 3H, J=6.6 Hz, CH3-C-6).

¹³CNMR (101 MHz, CDCl₃): δ=171.58 (COOAc), 170.19 (COOAc), 170.00 (COOAc), 169.99 (COOAc) 155.22 (NHCOOC(CH₃)₃), 98.66 (C-1′), 84.17, 83.74, 80.15, 74.54, 74.44, 71.44, 70.92, 69.15, 68.87, 61.74, 58.62, 48.99, 33.37, 28.27.

### Preparation of 3′,4′,6′,6-Tetra-O-acetate-2′,3-diazido-1-N-phenyl sulfonamide-6′-(R)-methyl-paromamine (Compound 9; FIG. 4)

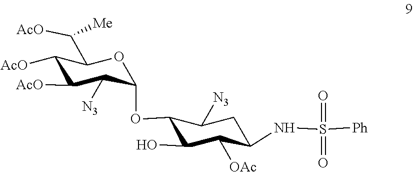

Compound 8 (0.460 gram, 0.7 mmol) was dissolved in dichloromethane (5 ml) and trifluoroacetic acid (1.5 ml) was added at ambient temperature. The reaction progress was monitored by TLC (EtOAc/Hexane 1:1), which indicated completion of the reaction after 1.5 hours. The reaction mixture was then concentrated to dryness under reduced pressure to afford 385 mg of N₁ free product. The crude product was dissolved chloroform (5 ml), followed by addition of N,N-Diisopropylethylamine (2.5 equiv, 0.25 ml) and benzenesulfonyl chloride (1.5 equivalent, 0.1 ml). The reaction progress was monitored by TLC (EtOAc/Hexane 3:7), which indicated completion of the reaction after 24 hours. The reaction mixture was thereafter concentrated to dryness under reduced pressure and purified by silica gel chromatography to afford Compound 9 (135 mg, 28%).

¹H NMR (500 MHz, CDCl₃): Ring I: δ=5.46 (dd,1H, J=10.4, 9.6 Hz, H-3), 5.37 (d,1H, J=3.5 Hz, H-1), 4.98-4.93 (m, 2H, H-4, H-6), 4.30 (dd,1H, J=10.5, 1.8 Hz, H-5), 3.56-3.52 (m, 1H, H-2), 1.22 (d,3H, J=6.0 Hz, CH₃—C-6). Ring II: δ=5.53 (dd, 1H, J=8.2, 0.6 Hz, H-4), 4.71 (t, 1H, J=10.0 Hz, H-6), 3.67-3.60 (m, 1H, H-5), 3.43-3.34 (m, 1H, H-1), 3.34-3.26 (m, 1H, H-3), 2.27 (dt, J=12.6, 4.1 Hz, 1H, H-2 eq), 1.58 (m, 1H, H-2 ax). Additional peaks in the spectrum were identified as follow: δ=7.86-7.83 (m, 2H, Ph), 7.62-7.58 (m, 1H, Ph), 7.53 (dd, 2H, J=10.4, 4.7 Hz, Ph), 2.07 (s, 3H, Ac), 2.06 (s, 3H, Ac), 2.03 (s, 3H, Ac), 1.81 (s, 3H, Ac).

¹³C NMR (126 MHz, CDCl₃): δ=172.01 (Carbonyl), 170.19 (Carbonyl), 170.01 (Carbonyl), 169.94 (Carbonyl), 140.88 (Ph 4°), 132.83 (Ph), 129.31 (Ph), 126.69 (Ph), 98.50 (C-1′), 82.60 (C-4), 74.23 (C-6), 74.13 (C-5), 71.16 (C-3′), 70.82 (C-5′), 69.01 (C-6′), 68.72 (C-4′), 61.51 (C-2′), 58.29 (C-3), 52.05 (C-1), 34.29 (C-2), 21.01 (Ac), 20.69 (Ac), 20.66 (Ac), 20.63 (Ac), 13.87 (CH₃—C-6′).

MALDI TOFMS: C₂₇H₃₅N₇O₁₃S ([M+Na]⁺) m/e 697.67; measured m/e 720.048.

### Preparation of 5-O-(5″-Azido-2″,3″-O-dibenzoyl-5″-deoxy-11-D-ribofuranose)-3′,4′,6′,6-tetra-O-acetate-2′,3-diazido-1-N-phenylsulfonamide-6′-(R)-methyl-paromamine (Compound 12; FIG. 5)

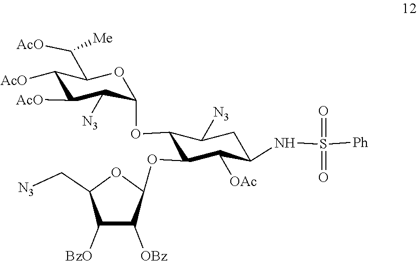

To powdered, flame-dried 4 Å molecular sieves was added freshly distilled CH₂Cl₂ (5 ml), followed by the addition of Compound 9 (358 mg, 0.513 mmol) and Donor Compound 10 (1 gram, 2.05 mmol), prepared as described in Fridman, M. et al. Angew. Chemie, Int. Ed. 44, 447-452 (2005). The mixture was stirred for 10 minutes at room temperature, and then cooled down to −30° C. A catalytic amount of BF₃.Et₂O was added and the mixture was allowed to reach room temperature while stirring. Propagation of the reaction was monitored by TLC (EtOAc/Hexane 3:7), which indicated completion after 2 hours. The reaction mixture was then diluted with EtOAc and filtered through Celite®. After through washing of the Celite® with EtOAc, the washes were combined and concentrated. The crude product was purified by flash chromatography to yield Compound 12 (314 mg, 57%).

¹HNMR (500 MHz, CDCl₃): Ring I: δ=5.90 (d,1H, J=3.8 Hz, H-1), 5.42 (dd, 1H, J=10.9, 9.2 Hz, H-3), 5.00-4.93 (m, 1H,H-4), 4.43 (dd,1H, J=10.5, 1.8 Hz, H-5), 3.44-3.38 (m, 1H, H-2), 1.24 (d, 3H, J=5.9 Hz, CHs-C-6). Ring II: 6=5.24 (d,1H, J=8.7 Hz, R₁NHSO₂R₂), 4.81 (dd, 1H, J=9.9, 9.3 Hz, H-5), 3.90-3.86 (m, 1H,H-4), 3.72-3.67 (m, 1H, H-6), 3.52-3.39 (m, 2H,H-1,H-3), 2.33 (dt, 1H, J=11.8, 3.8 Hz, H-2 eq), 1.56-1.46 (m, 1H, H-2 ax). Ring III: δ=5.59 (d, 1H, J=1.5 Hz, H-1), 5.49 (dd,1H, J=5.0, 1.1 Hz, H-2), 5.41 (dd,1H, J=7.1, 4.7 Hz, H-3), 4.48-4.44 (m, 1H, H-4), 3.61-3.58 (m, 1H, H-5). Additional peaks in the spectrum were identified as follow: δ=7.93-7.90 (m, 2H, Ph), 7.86-7.81 (m, 4H, Ph), 7.59 (dd, 2H, J=10.6, 4.3 Hz, Ph), 7.55-7.49 (m, 3H, Ph), 7.41 (t,2H, J=7.8 Hz, Ph), 7.33 (t, 2H, J=7.8 Hz, Ph), 2.08 (s, 3H, Ac), 2.07 (s, 3H, Ac), 2.04 (s, 3H, Ac), 1.82 (s, 3H, Ac). ¹³C NMR (126 MHz, CDCl₃): δ=171.62 (Carbonyl), 170.15 (Carbonyl), 170.00 (Carbonyl), 169.92 (Carbonyl), 165.24 (Carbonyl), 165.16 (Carbonyl), 140.84 (Ph4°), 133.75 (Ph), 133.63 (Ph), 132.92 (Ph), 129.68 (Ph), 129.63 (Ph), 129.37 (Ph), 128.68 (Ph), 128.57 (Ph), 128.50 (Ph), 128.43 (Ph), 126.68 (Ph), 107.22 (C-1″), 96.26 (C-1′), 80.24 (C-4), 80.20 (C-4″), 76.96 (C-6), 74.62 (C-2″), 73.48 (C-5), 71.62 (C-3″), 70.53 (C-3′), 70.24 (C-5′), 68.94 (C-6′), 68.53 (C-4′), 61.38 (C-2′), 58.73 (C-1), 52.78 (C-5″), 52.55 (C-3), 34.68 (C-2), 21.17 (Ac), 20.72 (Ac), 20.67 (Ac), 20.57 (Ac), 13.44 (CH₃—C-6′).

MALDI TOFMS: C₄₆H₅₀N₁₀O₁₈S ([M+Na]⁺) m/e 1063.01; measured m/e 1085.13.

### Preparation of 5-O-(5″-Azido-2″,3″-O-dibenzoyl-5″-deoxy-β-D-ribofuranose)-3′,4′,6′,6-tetra-O-acetate-2′,3-diazido-1-N-acetamide-6′-(R)-methyl-paromamine (Compound 13; FIG. 5)

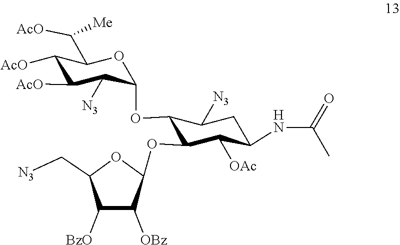

Compound 13 was prepared as described for the preparation of Compound 12 using Compound 7 as the starting material (398 mg, 0.663 mmol), CH₂Cl₂ (10 ml), and Donor 10 (1 gram, 1.722 mmol), affording 485 mg (75%).

¹HNMR (500 MHz, CDCl₃): Ring I: δ=5.94 (d, 1H, J=3.8 Hz, H-1), 5.47-5.41 (m, 1H, H-3), 5.02-4.94 (m, 2H, H-5, H-6), 4.48 (dd, 1H, J=10.6, 1.8 Hz, H-4), 3.47-3.41 (m, 1H, H-2), 1.26 (d, 3H,J=6.2 Hz, CH₃—C-6). Ring II: δ=5.79 (d,1H, J=8.0 Hz, RNHCO), 4.89-4.84 (m, 1H, H-4), 4.09 (ddd, 1H, J=9.5, 8.3, 5.2 Hz, H-1), 3.98 (t, 1H,J=9.1 Hz, H-5), 3.75-3.69 (m, 1H, H-6), 3.64-3.55 (m, 1H, H-3), 2.47 (dt,1H, J=8.7, 4.6 Hz, H-2 eq), 1.38 (t, 1H, J=12.8 Hz, H-2 ax). Ring III: δ=5.64 (d, 1H,J=1.5 Hz, H-1), 5.60 (d, 1H, J=5.9 Hz, H-2), 5.48 (dd, 1H, J=7.3, 4.8 Hz, H-3), 4.52-4.48 (m, 1H, H-4), 3.64-3.60 (m, 2H, H-5).

¹³CNMR (126 MHz, CDCl₃): δ=172.05 (Amide), 170.09 (Carbonyl), 170.02 (Carbonyl), 169.88 (Carbonyl), 169.79 (Carbonyl), 165.24 (Carbonyl),165.21 (Carbonyl), 133.72 (Ph), 133.64 (Ph), 129.72 (Ph), 129.66 (Ph), 128.70 (Ph), 128.56 (Ph), 128.53 (Ph), 128.44 (Ph), 107.46 (C-1″), 96.30 (C-1′), 80.44 (C-5), 80.15 (C-4″), 77.20 (C-6), 74.65 (C-2″), 74.06 (C-4), 71.56 (C-3″), 70.62 (C-4′), 70.21 (C-3′), 68.98 (C-5′), 68.62 (C-6′), 61.44 (C-2′), 58.84 (C-3), 52.75 (C-5″), 48.24 (C-4), 32.94 (C-2), 23.18 (RNHAc), 21.19 (Ac), 20.88 (Ac), 20.75 (Ac), 20.68 (Ac), 13.58 (CH₃—C-6′).

MALDI TOFMS: C₄₂H₄₈N₁₀O₁₇ ([M+Na]⁺) m/e 964.89; measured m/e 987.15.

### Preparation of 5-O-(5″-Azido-2″,3″-O-dibenzoyl-5″-deoxy-11-D-ribofuranose)-3′,4′,6′,6-tetra-O-acetate-2′,3-diazido-1-N-methanesulfonamide-6′-(R)-methyl-paromamine (Compound 14; FIG. 5)

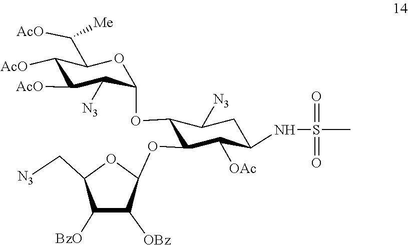

Compound 14 was prepared as described for the preparation of Compound 12, using Compound 6 (0.680 gram, 1 mmol) as the starting material, CH₂Cl₂ (10 ml), and Donor 10 (2 grams, 4 mmol), affording 0.73 gram (73%).

¹H NMR (500 MHz, CDCl₃): Ring I: δ=5.91 (d,1H, J=3.7 Hz, H-1), 5.46-5.40 (m,1H,H-3), 5.01-4.94 (m, 2H, H-4, H-6), 4.44 (dd, 1H, J=10.5, 1.6 Hz, H-5), 3.45 (dd, 1H, J=10.7, 4.1 Hz, H-2), 1.25 (d,1H, J=5.7 Hz, CH₃—C-6). Ring II: δ=5.06 (d,1H, J=9.5 Hz, NHSO₂—CH₃), 4.92 (t, 1H, J=9.7 Hz, H-6), 3.95 (t, 1H, J=8.9 Hz, H-5), 3.79-3.73 (m, 1H, H-4), 3.61-3.49 (m, 2H,H-1, H-3), 2.47 (dt, 1H, J=12.3, 4.0 Hz, H-2), 1.60 (dd, 1H, J=26.6, 12.0 Hz, H-2). Ring III: δ=5.64 (d, 1H,J=0.9 Hz, H-1), 5.59 (d, 1H,J=5.7 Hz, H-2), 5.45 (dd,1H, J=7.1, 4.7 Hz, H-3), 4.51 (d, 1H, J=7.0 Hz, H-4), 3.62 (d, 2H,J=4.5 Hz, H-5, H-5). Additional peaks in the spectrum were identified as follow: δ=7.93 (d, 2H, J=7.4 Hz, Ar), 7.86 (d, 2H, J=7.4 Hz, Ar), 7.55 (dt, 2H,J=23.6, 7.5 Hz, Ar), 7.41 (t,2H, J=7.8 Hz, Ar), 7.34 (t, 2H, J=7.8 Hz, Ar), 2.97 (s, 3H, NHSO₂—CH₃), 2.25 (s, 2H, Ac), 2.08 (s, 3H, Ac), 2.07 (s, 3H,Ac), 2.05 (s, 3H, Ac).

¹³C NMR (126 MHz, CDCl₃): δ=171.45 (Carbonyl), 170.12 (Carbonyl), 169.97 (Carbonyl), 169.86 (Carbonyl), 165.33 (Carbonyl), 165.19 (Carbonyl), 133.73 (Ar), 133.63 (Ar), 129.70 (Ar), 129.65 (Ar), 128.71 (Ar), 128.56 (Ar), 128.54 (Ar), 128.45 (Ar), 107.40 (C-1″), 96.28 (C-1′), 80.39 (C-4), 80.05 (C-6′), 77.09 (C-6), 74.66 (C-3), 73.47 (C-2″), 71.71 (C-5′), 70.61 (C-4′), 70.30 (C-3′), 68.99 (C-3″), 68.56 (C-4″), 61.46 (C-2′), 58.84 (C-1), 52.83 (C-3), 52.14 (C-5″), 42.08 (NHSO₂—CH₃), 34.82 (C-2), 21.15 (Ac), 21.06 (Ac), 20.70 (Ac), 20.64 (Ac), 13.50 (CH₃—C-6′).

MALDI TOFMS: C₄₁H₄₈N₁₀O₁₈S ([M+Na]⁺) m/e 1000.94; measured m/e 1023.31.

### Preparation of 5-O-(5″-Azido-2″,3″-O-dibenzoyl-5″-deoxy-11-D-ribofuranose)-3′,4′,6′,6-tetra-O-acetate-2′,3-diazido-1-N-benzamide-6′-(R)-methyl-paromamine (Compound 15; FIG. 5)

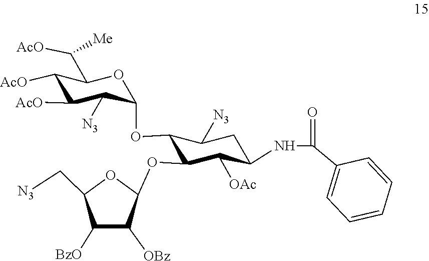

Compound 15 was prepared as described for the preparation of Compound 12, using Compound 5 (0.35 gram, 0.529 mmol) as the starting material, anhydrous CH₂Cl₂ (10 ml), and Donor 10 (0.864 gram, 2.11 mmol), affording 0.4 gram (73%).

¹HNMR (500 MHz, CDCl₃): Ring I: δ=5.98 (d,1H, J=3.6 Hz, H-1), 5.49-5.43 (m, 1H, H-3), 5.04-4.96 (m, 2H, H-4, H-6), 4.51 (d, 1H, J=9.8 Hz, H-5), 3.48-3.43 (m, 1H, H-2), 1.27 (d, 1H, J=5.7 Hz, CH₃—C-6). Ring II: δ=6.59 (d, 1H, J=7.6 Hz, amide), 5.02 (t,1H, J=9.8 Hz, H-6), 4.31-4.24 (m, 1H, H-1), 4.06 (t,1H, J=8.9 Hz, H-5), 3.81-3.73 (m, 1H, H-4), 3.66 (tt,1H. J=9.7, 4.8 Hz, H-3), 2.64 (dd, 1H, J=10.1, 3.1 Hz, H-2 eq), 1.51-1.42 (m, 1H, H-2 ax). Ring III: δ=5.69 (s, 1H, H-1), 5.64 (d, 1H, J=4.7 Hz, H-2), 5.51 (dd, 1H, J=6.5, 4.8 Hz, 1H), 4.51 (d,1H, J=7.4 Hz, H-3), 3.64 (m, 2H, H-5). Additional peaks in the spectrum were identified as follow: δ=7.94 (d, 2H, J=7.6 Hz, Ph), 7.85 (d,2H, J=7.7 Hz, Ph), 7.71 (d, 2H, J=7.5 Hz, Ph), 7.58 (t, 1H,J=7.3 Hz, Ph), 7.55-7.49 (m, 2H, Ph), 7.47-7.39 (m, 4H, Ph), 7.33 (t,2H, J=7.7 Hz, Ph), 2.15 (s, 3H, Ac), 2.09 (s, 3H, Ac), 2.08 (s, 3H, Ac), 2.06 (s, 3H, Ac).

¹³CNMR (126 MHz, CDCl₃): δ=172.55 (Amide), 170.08 (Carbonyl), 170.01 (Carbonyl), 169.85 (Carbonyl), 166.80 (Carbonyl), 165.22 (Carbonyl), 165.21 (Carbonyl), 133.69 (Ph), 133.61 (Ph), 133.21 (Ph), 131.96 (Ph), 129.71 (Ph), 129.65 (Ph), 128.74 (Ph), 128.55 (Ph), 128.42 (Ph), 126.83 (Ph), 107.55 (C-1″), 96.37 (C-1′), 80.64 (C-5), 80.13 (C-4″), 77.32 (C-4), 74.69 (C-2″), 74.23 (C-6), 71.56 (C-3″), 70.66 (C-5′), 70.25 (C-3′), 69.03 (C-6′), 68.63 (C-4′), 61.50 (C-2′), 58.94 (C-3), 52.77 (C-5″), 48.97 (C-1), 33.04 (C-2), 21.17 (Ac), 20.92 (Ac), 20.73 (Ac), 20.66 (Ac), 13.58 (CH₃—C-6′).

MALDI TOFMS: C₄₇H₅₀N₁₀O₁₇ ([M+H]⁺) m/e 1026.34; measured m/e 1027.28.

### Preparation of 5-O-(5″-(S)-Azido-2″,3″-O-dibenzoyl-5″-deoxy-β-D-ribofuranose)-3,4′,6′,6-tetra-O-acetate-2′,3-diazido-1-N-methanesulfonamide-6′-(R)-methyl-paromamine (Compound 16; FIG. 5)

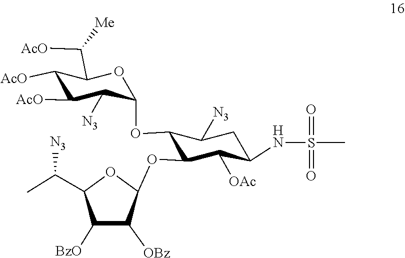

Compound 16 was prepared as described for the preparation of Compound 12, using Compound 6 (0.4 gram, 0.629 mmol) as the starting material, anhydrous CH₂Cl₂ (15 ml) and Donor Compound 11 (1.4 gram, 2.5 mmol), prepared as described in Kandasamy, J., et al. Medchemcomm 2, 165-171 (2011), affording 0.37 gram (58%).

¹HNMR (500 MHz, CDCl₁₃): Ring I: δ=5.92 (d, 1H, J=3.8 Hz, H-1), 5.44-5.37 (m, 1H, H-3), 4.97 (dd, 2H, J=17.2, 7.6 Hz, H-4, H-6), 4.43 (d, 1H, J=10.3 Hz, H-5), 3.53 (dd, 1H, J=10.8, 3.9 Hz, H-2), 1.24 (d, 3H, J=5.6 Hz, CH₃—C-6). Ring II: δ=5.11 (d,1H, J=9.4 Hz, NHSO₂—CH₃), 4.92 (t, 1H, J=9.6 Hz, H-5), 3.92 (t, 1H, J=8.9 Hz, H-6), 3.76 (t, 1H, J=9.1 Hz, H-4), 3.55 (ddd, 2H, J=17.4, 10.2, 5.3 Hz, H-1,H-3), 2.48 (dd, 1H, J=8.1, 4.0 Hz, H-2 eq), 1.61 (dd, 1H, J=26.0, 12.3 Hz, H-2 ax). Ring III: δ=5.61 (d, 2H, J=4.5 Hz, H-1, H-2), 5.48 (dd, 1H,J=7.8, 4.7 Hz, H-3), 4.31 (t, 1H, J=6.8 Hz, H-4), 3.72 (p, 1H, J=6.9 Hz, H-5) ,1.28 (d, 3H,J=7.0 Hz, CH₃—C-5′). Additional peaks in the spectrum were identified as follow: δ=7.89 (dd, 2H, J=27.3, 7.7 Hz, Ar), 7.55 (dt, 2H, J=20.8, 7.4 Hz, Ar), 7.37 (dt, 2H, J=30.8, 7.7 Hz, Ar), 2.98 (s, 3H, NHSO₂—CH₃), 2.32 (s, 3H, Ac) ,2.08 (s, 3H, Ac), 2.07 (s, 3H, Ac), 2.05 (s, 3H, Ac).

¹³C NMR (126 MHz, CDCl₃): δ=171.65 (Carbonyl), 170.17 (Carbonyl), 170.03 (Carbonyl), 169.86 (Carbonyl), 165.35 (Carbonyl), 165.01 (Ar), 133.73 (Ar), 133.64 (Ar), 129.71 (Ar), 129.63 (Ar), 128.74 (Ar), 128.68 (Ar), 128.56 (Ar), 128.51 (Ar), 128.45 (Ar), 107.60 (C-1″), 96.14 (C-1′), 83.99 (C-4″), 79.92 (C-6), 77.38 (C-4), 74.72 (C-2″), 73.15 (C-5), 71.62 (C-3″), 70.78 (C-3′), 70.22 (C-5′), 69.00 (C-4′), 68.56 (C-6′), 61.67 (C-2′), 59.02 (C-5″), 58.72 (C-1), 52.30 (C-3), 42.04 (NHSO₂—CH₃), 34.85 (C-2), 21.17 (Ac), 21.11 (Ac), 20.72 (Ac), 20.66 (Ac), 15.49 (CH₃—C-5′), 13.48 (CH₃—C-6′).

MALDI TOFMS: C₄₂H₅₀N₁₀O₁₈S ([M+Na]⁺) m/e 1014.97; measured m/e 1037.28.

### Preparation of 5-O-(5″-(S)-Azido-2″,3″-O-dibenzoyl-5″-deoxy-11-D-ribofuranose)-3′,4′,6′,6-tetra-O-acetate-2′,3-diazido-1-N-acetamide-6′-(R)-methyl-paromamine (Compound 17; FIG. 5)

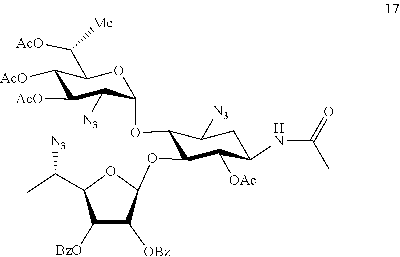

Compound 17 was prepared as described for the preparation of Compound 12 using Compound 7 (1.5 gram, 2.5 mmol) as the starting material, anhydrous CH₂Cl₂ (15 ml), and Donor 11 (4.5 grams, 10 mmol), affording 1.6 gram (64%).

¹H NMR (500 MHz, CDCl₃): Ring I: δ=5.95 (d, 1H, J=3.6 Hz, H-1), 5.41 (dd, 1H, J=10.6, 8.8 Hz, H-3), 5.01-4.93 (m, 2H, H-6, H-4), 4.46 (dd, 1H,J=10.6, 1.3 Hz, H-5), 3.50 (dd, 1H, J=10.6, 3.8 Hz, H-2), 1.25 (d,3H, J=6.0 Hz, CH₃—C-6). Ring II: δ=5.93 (d,1H, J=8.3 Hz, NH—Ac), 4.87 (t,1H, J=9.8 Hz, H-6), 4.14-4.04 (m, 1H, H-1), 3.94 (t, 1H, J=8.9 Hz, H-5), 3.79-3.68 (m, 1H, H-4), 3.60 (dt, 1H,J=11.6, 5.2 Hz, H-3), 2.48-2.41 (m, 1H, H-2 eq), 1.39 (dd,1H, J=26.8, 13.1 Hz, H-2 ax). Ring III: δ=5.61 (d, 2H, J=4.4 Hz, H-1, H-2), 5.51 (dd, 1H, J=7.9, 4.5 Hz, H-3), 4.31-4.27 (m, 1H, H-4), 3.70 (m, 1H, H-5), 1.29 (d, 3H, J=6.9 Hz, CH₃—C-5′). Additional peaks in the spectrum were identified as follow: δ=7.88 (dd, 4H,J=33.7, 7.4 Hz, Ar), 7.54 (dt, 2H, J=24.3, 7.4 Hz, Ar), 7.36 (dt,4H, J=36.9, 7.8 Hz, Ar), 2.22 (s, 3H,Ac), 2.08 (s, 3H, Ac), 2.07 (s, 3H, Ac), 2.04 (s, 3H, Ac), 1.94 (s, 3H, Ac).

¹³C NMR (126 MHz, CDCl₃): δ=172.16 (Carbonyl), 170.09 (Carbonyl), 170.01 (Carbonyl), 169.82 (Carbonyl), 169.7 (Carbonyl), 165.23 (Carbonyl), 165.04 (Carbonyl), 133.68 (Ar), 133.60 (Ar), 129.71 (Ar), 129.67 (Ar), 129.63 (Ar), 128.69 (Ar), 128.54 (Ar), 128.42 (Ar), 107.60 (C-1″), 96.13 (C-1′), 83.64 (C-4″), 80.41 (C-5), 77.54 (C-4), 74.78 (C-2″), 73.82 (C-6), 71.47 (C-3″), 70.82 (C-5′), 70.17 (C-3′), 69.03 (C-6′), 68.63 (C-4′), 61.64 (C-2′), 58.82 (C-5″), 48.23 (C-1), 32.80 (C-2), 23.15 (Ac), 21.14 (Ac), 20.89 (Ac), 20.71 (Ac), 20.65 (Ac), 15.51 (CH₃—C-5″), 13.57 (CH₃—C-6′).

MALDI TOFMS: C₄₃H₅₀N₁₀O₁₇([M+Na]⁺) m/e 978.91; measured m/e 1001.32.

### Preparation of 5-O-(5″-(S)-Azido-2″,3″-O-dibenzoyl-5″-deoxy-11-D-ribofuranose)-3′,4′,6′,6-tetra-O-acetate-2′,3-diazido-1-N-benzamide-6′-(R)-methyl- paromamine (Compound 18; FIG. 5)

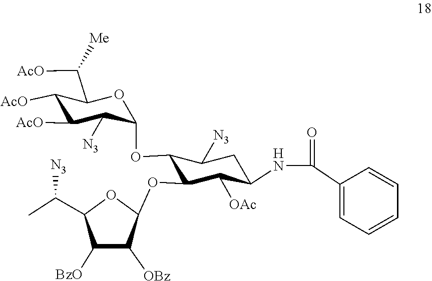

Compound 18 was prepared as described for the preparation of Compound 12, using Compound 5 (0.323 gram, 0.488 mmol) as the starting material, anhydrous CH₂Cl₂ (10 ml), and Donor 11 (1.4 gram, 1 mmol), affording 0.422 gram (83%).

¹NMR (500 MHz, CDCl₃): Ring I: δ=5.99 (d, 1H, J=3.8 Hz, H-1), 5.44 (t,1H, J=10.3 Hz, H-3), 5.00 (dd, 2H, J=19.0, 8.7 Hz, H-5, H-6), 4.49 (dd, 1H, J=10.3, 1.3 Hz, H-4), 3.53 (dd, 1H, J=10.6, 4.1 Hz, H-2), 1.26 (d, 3H, J=5.8 Hz, CH₃—C-6). Ring II: δ=6.62 (d, 1H, J=7.8 Hz, NHCOBz), 5.03 (d, 1H, J=10.1 Hz, H-6), 4.33-4.23 (m, 1H,H-1), 4.02 (t, 1H,J=8.9 Hz, H-5), 3.77 (t, 1H, J=9.2 Hz, H-4), 3.69-3.62 (m, 1H, H-3), 2.64 (dt, 1H, J=7.9, 5.1 Hz, H-2 eq), 1.46 (q, 1H, J=12.2 Hz, H-2 ax). Ring III: δ=5.67 (d, 2H, J=4.5 Hz, H-1, H-2), 5.54 (dd, 1H, J=7.8, 4.9 Hz, H-3), 4.34-4.27 (m, 1H, H-4), 3.72 (p, 1H, J=7.0 Hz, H-5), 1.31 (d, 3H, J=6.9 Hz, CH₃—C-5′). Additional peaks in the spectrum were identified as follow: δ=7.92 (d, 2H, J=7.5 Hz, Ar), 7.84 (d, 2H, J=7.6 Hz, Ar), 7.72 (d, 2H, J=7.4 Hz, Ar), 7.57 (t, 1H, J=7.4 Hz, Ar), 7.52 (t,2H, J=7.4 Hz, Ar), 7.43 (dt, 4H, J=19.0, 7.6 Hz, Ar), 7.33 (t, 2H, J=7.8 Hz, Ar), 2.22 (s, 3H, Ac), 2.09 (s, 3H, Ac), 2.08 (s, 3H, Ac), 2.06 (s, 3H, Ac).

¹³C NMR (126 MHz, CDCl₃): δ=172.81 (Carbonyl), 170.10 (Carbonyl), 170.03 (Carbonyl), 169.82 (Carbonyl), 166.79 (Carbonyl), 165.21 (Carbonyl), 165.02 (Carbonyl), 133.66 (Ar), 133.58 (Ar), 133.25 (Ar), 131.95 (Ar), 129.72 (Ar), 129.63 (Ar), 128.74 (Ar), 128.53 (Ar), 128.41 (Ar), 126.84 (Ar), 107.74 (C-1″), 96.23 (C-1′), 83.67 (C-4″), 80.55 (C-5), 77.64 (C-4), 74.84 (C-2″), 73.91 (C-6), 71.46 (C-3″), 70.85 (C-4′), 70.20 (C-4′), 69.05 (C-6′), 68.66 (C-5′), 61.70 (C-2′), 58.90 (C-5″), 58.88 (C-3), 49.09 (C-1), 32.96 (C-2), 21.17 (Ac), 20.97 (Ac), 20.73 (Ac), 20.66 (Ac), 15.53 (CHs-C-5′), 13.60 (CH₃—C-6′).

MALDI TOFMS: C₄₇H₅₀N₁₀O₁₇ ([M+H₂O]⁺) m/e 1046.98; measured m/e 1065.6.

### Preparation of 6 (R)-Methyl-5-O-(5″-azido-5″-deoxy-11-D-ribofuranose)-2-azido-1-N-phenylsulfonamide paromamine (Compound 19; FIG. 5)

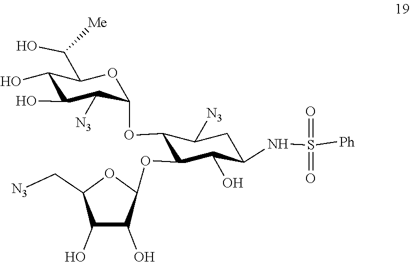

Compound 12 (314 mg, 0.295 mmol) was treated with a solution of MeNH₂ (33% solution in EtOH, 5 ml). The propagation of the reaction was monitored by TLC (EtOAc/MeOH, 7:3), which indicated completion after 12 hours. The reaction mixture was then evaporated to dryness and purified by silica gel chromatography (100% EtOAc) to afford Compound 19 (0.148 gram, 73%).

¹HNMR (500 MHz, MeOD): Ring I: δ=6.00 (d,1H, J=3.6 Hz, H-1), 4.02 (dd,1H, J=6.8, 3.5 Hz, H-5), 3.94-3.86 (m, 2H, H-3,H-6), 3.32 (dd,1H, J=10.0, 8.7 Hz, H-4), 3.11 (dd, 1H, J=10.6, 4.3 Hz, H-2), 1.21 (d, 1H, J=6.0 Hz, CHs-C-6). Ring II: δ=3.60 (m, 2H, J=17.3, 9.0 Hz, H-4, H-5), 3.43-3.35 (m, 1H, H-1), 3.27-3.13 (m, 2H, H-3, H-6), 1.85 (dt, 1H, J=12.3, 4.0 Hz, H-2 eq), 1.33-1.20 (m, 1H, H-2 ax). Ring III: δ=5.29 (d, 1H, J=0.8 Hz, H-1), 4.14 (d, 1H, J=5.1 Hz, H-2), 4.02 (dd, 1H, J=7.5, 4.1 Hz, H-3), 3.98 (td, 1H, J=6.3, 2.9 Hz, H-4), 3.55 (dd, 1H, J=13.3, 2.7 Hz, H-5), 3.46 (dd, 1H, J=13.2, 6.2 Hz, H-5). Additional peaks in the spectrum were identified as follow: δ=7.92 (dd, 2H J=5.2, 3.4 Hz, Ph), 7.63 (ddd, 1H, J=8.6, 2.4, 1.2 Hz, Ph), 7.60-7.54 (m, 2H, Ph).

¹³CNMR (126 MHz, MeOD): δ=142.82 (Ph 4°), 133.63 (Ph), 130.16 (Ph), 127.95 (Ph), 111.21 (C-1″), 97.10 (C-1′), 85.77 (C-5), 82.13 (C-4″), 76.39 (C-4), 76.24 (s), 76.00 (C-6), 75.01 (C-3′), 74.10 (C-4′), 72.57 (C-5′), 72.46 (C-6′), 68.97 (C-3″), 64.85 (C-2′), 61.38 (C-1), 54.82 (C-3), 54.39 (C-5″), 34.40 (C-2), 17.65 (CH₃—C-6′).

### Preparation of 6′-(R)-Methyl-5-O-(5″-azido-5″-deoxy-11-D-ribofuranose)-2′,3-azido N-acetamide paromamine (Compound 20; FIG. 5)

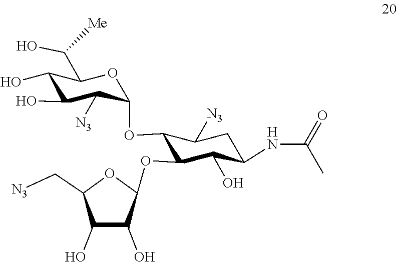

Compound 20 was prepared as described for the preparation of Compound 19 using Compound 13 (485 mg, 0.824 mmol) as the starting material, and a solution of MeNH₂ (33% solution in EtOH, 10 ml), affording 278 mg (93%).

¹H NMR (500 MHz, MeOD): Ring I: δ=5.99 (d,1H, J=3.6 Hz, H-1), 4.00 (dd, 1H,J=6.8, 3.6 Hz, H-6), 3.93 (dd,1H, J=9.9, 4.1 Hz, H-4), 3.88 (dd,1H, J=10.1, 9.1 Hz, H-3), 3.28 (dd,1H, J=10.1, 8.7 Hz, H-5), 3.07 (dd,1H, J=10.6, 4.3 Hz, H-2), 1.19 (d, 1H, J=6.0 Hz, CH₃—C-6). Ring II: δ=3.75 (ddd, 1H,J=12.0, 10.4, 4.5 Hz, H-1), 3.69-3.63 (m, 1H, H-4), 3.60 (t, 1H,J=8.9 Hz, H-5), 3.49 (ddd, 1H, J=12.3, 9.7, 4.8 Hz, H-3), 3.28 (dd, 1H, J=10.4, 8.9 Hz, m, 1H, H-6), 2.09 (dt, 1H, J=12.5, 4.3 Hz, H-2 eq), 1.32 (dd,1H, J=25.7, 12.6 Hz, H-2 ax). Ring III: δ=5.29 (d, 1H,J=1.1 Hz, H-1), 4.12 (d, 1H,J=5.3 Hz, H-2), 3.99 (dd,1H, J=7.5, 4.2 Hz, H-3), 3.95 (dd, 1H,J=7.0, 3.4 Hz, H-4), 3.52 (dd, 1H,J=13.2, 2.7 Hz, H-5), 3.44 (dd, 1H,J=13.2, 6.1 Hz, H-5).

¹³CNMR (126 MHz, MeOD): δ=173.48 (Amide), 111.24 (C-1″), 97.25 (C-1′), 86.33 (C-5), 82.27 (C-4″), 76.66 (C-2″), 76.36 (C-5′), 76.14 (C-4), 75.14 (C-4′), 74.20 (C-3″), 72.67 (C-6), 72.52 (C-3′), 69.03 (C-6′), 64.97 (C-2′), 61.96 (C-3), 54.48 (C-5″), 50.71 (C-1), 33.85 (C-2), 22.80 (Ac), 17.63 (CH₃—C-6′).

MALDI TOF MS: C₂₀H₃₂N₁₀O₁₁ ([M+Na]⁺) m/e 588.53; measured m/e 611.11.

### Preparation of 6′-(R)-Methyl-5-O-(5″-azido-5″-deoxy-11-D-ribofuranose)-2′,3-azido-1-N-methanesulfonamide paromamine (Compound 21; FIG. 5)

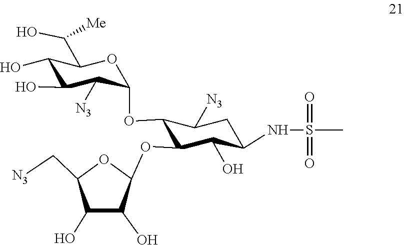

Compound 21 was prepared as described for the preparation of Compound 19 using Compound 14 (0.73 gram, 0.729 mmol) and a solution of MeNH₂ (33% solution in EtOH, 10 ml), affording 0.426 gram (93%).

¹HNMR (500 MHz, MeOD): Ring I: δ=6.03 (d, 1H, J=3.2 Hz, H-1), 4.06 (dd, 1H, J=7.0, 3.3 Hz, H-6), 4.00-3.91 (m, 2H, H-5, H-3), 3.39-3.32 (m, 1H, H-4), 3.14 (dd, 1H, J=10.7, 4.8 Hz, H-2), 1.26 (d, 3H, J=4.8 Hz, CHs-C-6). Ring II: δ=3.74-3.64 (m,2H, H-4, H-6), 3.60-3.52 (m, 1H, H-1), 3.30 (dd,1H, J=9.5, 4.6 Hz, H-3), 2.28 (dd, 1H, J=8.4, 2.9 Hz, H-2 eq), 1.46 (dd, 1H, J=26.2, 12.4 Hz, H-1 ax). Ring III: δ=5.37 (s, 1H, H-1), 4.19 (d,1H, J=4.2 Hz, H-2), 4.06 (t, 2H, J=5.5 Hz, H-3, H-4), 3.62-3.48 (m, 2H, H-5, H-5), Additional peaks in the spectrum were identified as follow: δ=3.06 (s, 3H, NHSO₂—CH₃).

¹³CNMR (126 MHz, MeOD): δ=109.65 (C-1″), 95.82 (C-1′), 84.58 (C-4), 80.93 (C-6′), 75.09 (C-6), 74.92 (C-3), 74.89 (C-2″), 73.70 (C-5′), 72.81 (C-4′), 71.20 (C-3′), 71.10 (C-3″), 67.68 (C-4″), 63.52 (C-2′), 60.33 (C-1), 53.50 (C-3), 53.03 (C-5″), 40.26 (NHSO₂—CH₃), 34.46 (C-2), 16.31 (CH₃—C-6′).

MALDI TOF MS: ([M−H]⁺) m/e 624.58; measured m/e 623.19.

### Preparation of 6′-(R)-Methyl-5-O-(5″-azido-5″-deoxy-11-D-ribofuranose)-2′,3-azido-1-N-benzamide paromamine (Compound 22; FIG. 5)

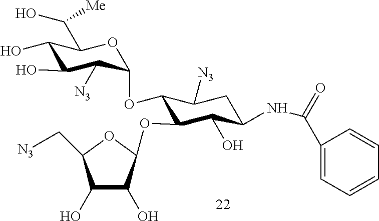

Compound 22 was prepared as described for the preparation of Compound 19 using Compound 15 (0.4 gram, 0.389 mmol) and a solution of MeNH₂ (33% solution in EtOH, 10 ml), affording 0.25 gram (95%).

¹HNMR (500 MHz, MeOD): Ring I: δ=6.08 (d, 1H, J=3.3 Hz, H-1), 4.07 (dd, 1H, J=7.1, 3.4 Hz, H-6), 4.03-3.95 (m, 2H,H-3,H-5), 3.42-3.37 (m, 1H, H-4), 3.16 (dd, 1H,J=10.6, 4.7 Hz, H-2), 1.28 (d, 1H,J=4.8 Hz, CH₃—C-6). Ring II: δ=8.35 (d, 1H, J=7.9 Hz, Amide), 4.08 (td, 2H, J=11.1, 4.8 Hz, H-1, H-5), 3.81-3.70 (m, 1H, H-4), 3.59 (dt, 2H,J=18.7, 7.0 Hz, H-6, H-3), 2.25 (dd, 1H, J=8.0, 4.0 Hz,H-2 eq), 1.57 (dd, 1H, J=26.2, 12.8 Hz, H-2 ax). Ring III: δ =5.41 (s, 1H, H-1), 4.22 (d,1H, J=5.2 Hz, H-2), 4.09 (dd,1H, J=7.8, 3.8 Hz, H-3), 4.06-4.02 (m, 1H,H-4), 3.59 (dd, 1H, J=13.1, 2.3 Hz, H-5), 3.51 (dd, 1H, J=13.1, 6.3 Hz, H-5). Additional peaks in the spectrum were identified as follow: δ=7.89 (d,2H, J=7.4 Hz, Ph), 7.56 (t, 1H, J=7.3 Hz, Ph), 7.48 (t,2H, J=7.6 Hz, Ph).

¹³CNMR (126MHz, MeOD): δ=169.01 (Amide), 134.16 (Ph), 131.39 (Ph), 128.11 (Ph), 127.06 (Ph), 109.87 (C-1″), 95.88 (C-1′), 84.94 (C-6), 80.91 (C-4″), 75.39 (s), 74.99 (C-2″), 74.51 (C-4), 73.71 (C-5′), 72.94 (C-4′), 71.25 (C-3″), 71.11 (C-3′), 67.86 (C-6″), 63.52 (C-2′), 60.67 (C-3), 53.10 (C-5″), 32.30 (C-2), 16.54 (CH₃—C-6′).

### Preparation of 6′-(R)-Methyl-5-O-(5″-(S)-azido-5″-deoxy-11-D-ribofuranose)-2′,3-azido-1-N-methanesulfonamide paromamine (Compound 23; FIG. 5)

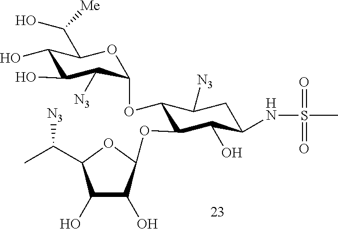

Compound 23 was prepared as described for the preparation of Compound 19 using Compound 16 (0.37 gram, 0.364 mmol) and a solution of MeNH₂ (33% solution in EtOH, 10 ml), affording 0.230 gram (98%).

¹H NMR (500 MHz, MeOD): Ring I: δ=6.04 (d,1H, J=3.6 Hz, H-1), 4.02 (dd, 1H, J=6.8, 3.3 Hz, H-6), 3.95-3.89 (m, 2H, H-3, H-5), 3.29 (dd, 1H, J=10.0, 8.7 Hz, H-4), 3.08 (dd, 1H, J=10.6, 4.4 Hz, H-2), 1.21 (d, 3H, J=6.0 Hz, CH₃—C-6). Ring II: δ 3.68 (t, 1H, J=9.6 Hz, H-4), 3.61 (t, 1H, J=8.5 Hz, H-5), 3.54-3.47 (m, 1H, H-1), 3.29-3.20 (m, 2H, H-3, H-6), 2.27-2.19 (m, 1H, H-2 eq), 1.42 (dd, 1H, J=27.1, 11.5 Hz, H-2 ax). Ring III: δ=5.30 (d, 1H, J=0.5 Hz, H-1), 4.14 (d, 1H, J=5.4 Hz, H-2), 4.08 (dd,1H, J=7.7, 4.2 Hz, H-3), 3.72 (t,1H, J=6.4 Hz, H-4), 3.67-3.60 (m, 1H, H-5), 1.33 (d, 3H, J=6.9 Hz, CH₃—C-5′). Additional peaks in the spectrum were identified as follow: δ=3.01 (s, 3H, NHSO₂—CH₃).

¹³CNMR (126MHz, MeOD): δ=109.27 (C-1″), 95.81 (C-1′), 84.65 (C-4″), 84.53 (C-5), 75.12 (C-2″), 74.94 (C-4), 74.83 (C-6), 73.73 (C-3′), 72.77 (C-4′), 71.27 (C-3″), 71.15 (C-5′), 67.51 (C-6″), 63.41 (C-2′), 60.37 (C-1), 59.27 (C-5″), 53.50 (C-3), 40.23 (NHSO₂—CH₃), 34.47 (C-2), 16.10 (CH₃—C-5′), 14.66 (CH₃—C-6′).

MALDI TOFMS: C₂₀H₃₄N₁₀O₁₂S ([M+H]⁻) m/e 638.61; measured m/e 637.5.

### Preparation of 6′-(R)-Methyl-5-O-(5″-(S)-azido-5″-deoxy-11-D-ribofuranose)-2′,3-azido-1-N-acetamide paromamine (Compound 24; FIG. 5)

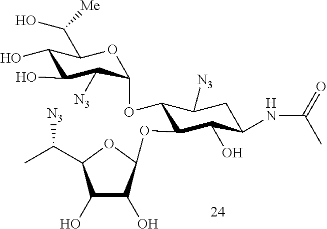

Compound 24 was prepared as described for the preparation of Compound 19 using Compound 17 (1.6 gram, 1.6 mmol) and a solution of MeNH₂ (33% solution in EtOH, 10 ml), affording 0.820 gram (81%).

¹H NMR (500 MHz, MeOD): Ring I: δ=6.07 (d,1H, J=3.4 Hz, H-1), 4.06-4.00 (m, 1H, H-6), 3.98-3.91 (m, 2H, H-3, H-5), 3.31 (dd, 1H, J=10.0, 8.8 Hz, H-4), 3.09 (dd, 1H,J=10.6, 4.5 Hz, H-2), 1.23 (d, 3H, J=5.0 Hz, CH₃—C-6). Ring II: δ=3.81-3.74 (m, 1H, H-1), 3.73-3.68 (m, 1H, H-4,), 3.68-3.61 (m, 1H, H-3), 3.56-3.48 (m, 1H, H-3), 3.32 (dd, 1H, J=10.0, 9.4 Hz, H-6), 2.12 (dt, 1H, J=12.1, 4.1 Hz, H-2 eq), 1.35 (dd, 1H, J=25.9, 12.6 Hz, H-2 ax). Ring III: δ=5.30 (s, 1H, H-1), 4.14 (d, 1H, J=5.3 Hz, H-2), 4.11-4.06 (m, 1H, H-3), 3.73 (t, 1H, J=5.8 Hz, H-4), 3.66 (dd, 1H, J=11.5, 3.6 Hz, H-5), 1.34 (d, 3H,J=6.7 Hz, CH₃—C-5′).

¹³CNMR (126 MHz, MeOD): δ=108.70 (C-1″), 95.10 (d,C-1′), 84.38 (C-5), 83.97 (C-4″), 74.56 (C-5″), 74.51 (C-2″), 74.04 (C-4), 73.12 (C-3′), 72.21 (C-6), 70.70 (C-3″), 70.52 (C-6′), 66.96 (C-6″), 62.81 (C-2′), 59.97 (C-3), 58.70 (s), 48.67 (C-1), 47.23 (C-4′), 31.82 (C-2), 20.80 (Ac), 15.51 (CH₃—C-6′), 14.05 (CH₃—C-5″).

MALDI TOFMS: C₂₁H₄₀N₄O₁₁([M+Na]⁺) m/e 602.56; measured m/e 625.23.

### Preparation of 6′-(R)-Methyl-5-O-(5″-(S)-azido-5″-deoxy-11-D-ribofuranose)-2′,3-azido-1-N-benzamide paromamine (Compound 25; FIG. 5)

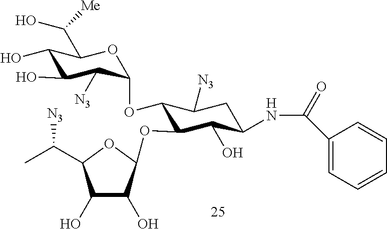

Compound 25 was prepared as described for the preparation of Compound 19 using Compound 18 (0.422 gram, 0.405 mmol) and a solution of MeNH₂ (33% solution in EtOH, 10 ml), affording 0.260 gram (96%).

¹HNMR (500 MHz, MeOD): Ring I: δ=6.11 (d, 1H, J=3.5 Hz, H-1), 4.07-4.03 (m, 1H, H-6), 4.01-3.93 (m, 2H, H-3, H-5), 3.33 (dd, 1H, J=10.0, 8.8 Hz, H-4), 3.14-3.07 (m, 1H, H-2), 1.24 (d, 3H, J=6.1 Hz, CHs-C-6). Ring II: δ=4.04 (ddd, 1H, J=11.1, 10.0, 3.8 Hz, H-1), 3.80-3.72 (m, 1H, H-4), 3.72-3.65 (m, 1H, H-5), 3.65-3.55 (m, 1H, H-3), 3.51 (dd, 1H, J=10.6, 8.2 Hz, H-6), 2.23 (dt, 1H, J=11.8, 3.6 Hz, H-2 eq), 1.57-1.47 (m, 1H, H-2 ax). Ring III: δ=5.33 (s, 1H, H-1), 4.17 (d, 1H, J=5.2 Hz, H-2), 4.14-4.07 (m, 1H, H-3), 3.76-3.72 (m, 1H, H-4), 3.66 (dd, 1H, J=6.4, 5.6 Hz, H-5), 1.35 (d, 3H, J=7.0 Hz, CH₃—C-5′). Additional peaks in the spectrum were identified as follow: δ=7.84 (d,2H, J=8.6 Hz, Ar), 7.54 (t,1H, J=7.4 Hz, Ar), 7.46 (t, 2H, J=7.6 Hz, Ar).

¹³C NMR (126 MHz, MeOD): δ=169.04 (Carbonyl), 134.16 (Ar), 131.53 (Ar), 128.05 (Ar), 126.97 (Ar), 109.54 (C-1″), 95.76 (C-1′), 85.02 (C-5), 84.82 (C-4″), 75.23 (C-2″), 75.15 (C-4), 74.46 (C-3′), 73.72 (C-4′), 72.86 (C-6), 71.32 (C-3″), 71.16 (C-5′), 67.61 (C-6′), 63.44 (C-2′), 60.69 (C-3), 59.34 (C-5″), 49.85 (C-1), 32.36 (C-2), 16.16 (CH₃—C-6′), 14.63 (CH₃—C-5′).

MALDI TOFMS: C₂₆H₃₆N₁₀O₁₁ ([M+Na]⁺) m/e 664.62; measured m/e 687.25.

### Preparation of 6′-(R)-Methyl-5-O-(5″-amino-5″-deoxy-II-D-ribofuranose)-2′,3-amino-1-N-phenylsulfonamide paromamine (NB74-N1PhS; FIGS. 2 and 5)

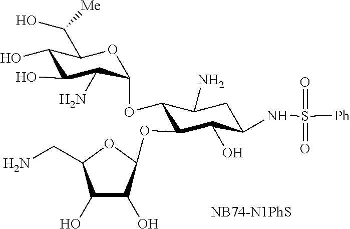

Compound 19 (0.148 gram, 0.243 mmol) was dissolved in a mixture of THF (5 ml) and aqueous NaOH (0.1 M, 1.5 ml). The mixture was stirred at room temperature for 10 minutes, after which PMe₃ (1M solution in THF 4 ml) was added. Propagation of the reaction was monitored by TLC [CH₂Cl₂/MeOH/H₂O/MeNH₂ (30% solution in EtOH), 10:15:6:15], which indicated completion after 5 hours. The reaction mixture was thereafter purified by flash chromatography on a silica gel column. The column was washed with EtOAc, THF, and MeOH. The product was eluted with the mixture of 20% MeNH₂ solution (30% solution in EtOH) in 80% MeOH. Fractions containing the product were combined and evaporated under vacuum; re-dissolved in small volume of water and passed through a short column of Amberlite CG50 (NH₄⁺ form). The column was first washed with H₂O, MeOH/H₂O, MeOH, and H₂O. Then the product was eluted with a mixture of MeOH/H₂O/NH₄OH (80:10:10) to afford the compound NB74-N1PhS (67 mg, 51%). For the storage and biological tests NB74-N1PhS was converted to its sulfate salt form: the free base was dissolved in water, and pH was adjusted to 7 with H₂SO₄ (0.1 N) and lyophilized.

¹H NMR (500 MHz, MeOD): Ring I: δ=5.16 (d, 1H, J=3.3 Hz, H-1), 4.04 (dd, 1H, J=5.1, 2.2 Hz, H-6), 3.74 (dd, 1H, J=9.9, 3.1 Hz, H-5), 3.52-3.45 (m, 1H, H-3), 3.19-3.13 (m, 1H, H-4), 2.59 (q, 1H, J=6.2 Hz, H-2), 1.15 (d, 3H, J=5.9 Hz, 1H, CH₃—C-6). Ring II: δ 3.49 (t, 1H, J=9.1 Hz, H-5), 3.35-3.27 (m, 2H, H-4, H-6), 3.17-3.08 (m, 1H, H-1), 2.74-2.64 (m, 1H, H-3), 1.84 (dd, 1H, J=8.7, 4.0 Hz, H-2), 1.23 (m,1H, H-2) Ring III: δ=5.17 (d, 1H, J=2.2 Hz, H-1), 4.06 (dd, 1H, J=6.6, 3.1 Hz,H-2), 3.91-3.88 (m, 1H,H-3), 3.88-3.83 (m, 1H, H-4), 2.96 (dd, 1H, J=13.2, 3.5 Hz, 1H,H-5), 2.77 (dd, 1H, J=13.2, 7.5 Hz, H-5). Additional peaks in the spectrum were identified as follow: δ=7.89-7.86 (m, 2H, Ph), 7.60-7.55 (m, 1H, Ph), 7.52 (dd, 2H, J=10.2, 4.7 Hz, Ph).

¹³CNMR (126 MHz, MeOD): δ=143.12 (Ph)4° , 133.54 (Ph), 130.15 (Ph), 127.96 (Ph), 111.18 (C-1″), 101.60 (C-1′), 85.81 (C-5), 85.69 (C-4), 83.47 (C-4″), 76.73 (C-6), 76.45 (C-5′), 76.34 (s), 74.92 (C-3′), 73.53 (C-4′), 72.69 (C-3″), 67.75 (C-6′), 57.54 (C-2′), 55.53 (C-1), 51.95 (C-3), 44.78 (C-5″), 36.75 (C-2), 16.55 (CH₃—C-6′). MALDI TOFMS C₂₂H₃₃N₇O₁₃S ([M+H]⁺) m/e 608.66; measured m/e 609.06).

### Preparation of 6′-(R)-Methyl-5-O-(5″-amino-5″-deoxy-β-D-ribofuranose)-2′,3-amino-1-N-acetamide paromamine (NB74-N1Ac; FIGS. 2 and 5)

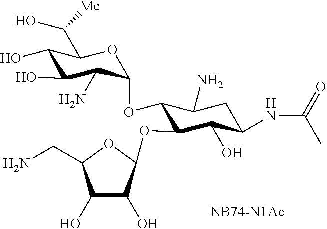

Compound NB74-N1Ac was prepared as described for the preparation of Compound NB74-N1PhS using Compound 20 (278 mg, 0.544 mmol), THF (5 ml), NaOH (0.1 M, 1.5 ml), and PMe₃ (1M solution in THF 4 ml), affording 74 mg (29%).

¹H NMR (500 MHz, MeOD): Ring I: δ=5.15 (d,1H, J=3.0 Hz, H-1), 4.09-4.01 (m, 1H, H-6), 3.77 (dd, 1H, J=9.9, 3.1 Hz,H-5), 3.49 (td, 1H, J=10.0, 1.5 Hz, H-3), 3.15 (dd,1H, J=9.5, 9.1 Hz, H-4), 2.62-2.55 (m, 1H, H-2), 1.14 (d, 3H, J=5.5 Hz, CH₃—C-6). Ring II: δ=3.75-3.67 (m, 1H,H-1), 3.53-3.45 (m, 1H, H-5), 3.37-3.28 (m, 2H,H-4, H-6), 2.81-2.73 (m, 1H,H-3), 1.91-1.90 (m, 1H,H-2), 1.25-1.13 (m, 1H, H-2).Ring III: δ=5.19 (d,1H, J=2.8 Hz, H-1), 4.04 (dd,1H, J=5.1, 2.3 Hz, H-2), 3.92-3.87 (m, 1H, H-3), 3.87-3.82 (m, 1H, H-4), 2.98-2.92 (m, 1H, H-5), 2.80-2.73 (m, 1H, H-5). Additional peaks in the spectrum were identified as follow: δ=1.90 (s, 3H, Ac).

¹³CNMR (126 MHz, MeOD): δ=173.41 (Amide), 111.13 (C-1″), 101.75 (C-1′), 86.18 (C-4), 86.05 (C-5), 83.65 (C-4″), 83.55 (C-3′), 76.82 (C-6), 76.49 (C-2″), 76.29 (C-5′), 73.55 (C-4′), 72.63 (C-3″), 67.68 (C-6′), 57.62 (C-2′), 52.26 (C-3), 51.18 (C-1), 44.73 (C-5″), 30.78 (C-2), 18.38 (Ac), 16.47 (CH₃—C-6′).

MALDI TOF MS: C₂₀H₃₂N₁₀O₁₁ ([M+H]⁺) m/e 510.54; measured m/e 511.14.

### Preparation of 6′-(R)-Methyl-5-O-(5″-amino-5″-deoxy-11-D-ribofuranose)-2′,3-amino-1-N-methanesulfonamide paromamine (NB74-N1MeS; FIGS. 2 and 5)

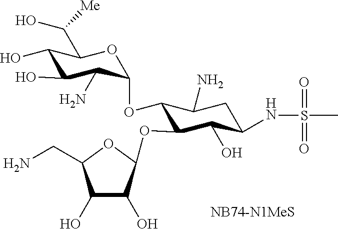

Compound NB74-N1MeS was prepared as described for the preparation of Compound NB74-N1PhS using Compound 21 (0.426 gram, 0.68 mmol), THF (10 ml), aqueous NaOH (0.1 M, 2 ml), and PMe₃ (1M solution in THF 2 ml), affording 150 mg (40%).

¹H NMR (500 MHz, MeOD): Ring I: δ=5.23 (d, 1H, J=2.8 Hz, H-1), 4.12 (dd, 1H, J=6.2, 2.6 Hz, H-6), 3.82 (dd,1H, J=10.0, 2.9 Hz, H-5), 3.58-3.52 (m, 1H, H-3), 3.25-3.16 (m, 1H, H-4, 2.69-2.64 (m, 1H, H-4), 1.20 (d, 3H, J=1.0 Hz, CHs-C-6). Ring II: δ=3.58-3.52 (m, 1H,H-5), 3.34 (dt, 2H, J=18.2, 8.3 Hz, H-4, H-6), 3.28-3.21 (m, 1H, H-1), 2.90-2.78 (m, 1H, H-3), 2.15-2.09 (m, 1H, H-2 eq), 1.38-1.29 (m, 1H, H-2, ax). Ring III: δ=5.29 (d, 1H, J=2.4 Hz, H-1), 4.13-4.10 (m, 1H, H-2), 3.98-3.94 (m, 2H, H-3, H-4), 3.09 (dd,1H, J=12.6, 2.6 Hz, H-5), 2.92-2.84 (m, 1H, H-5). Additional peaks in the spectrum were identified as follow: δ=3.02 (s, 3H, NHSO₂—CH₃).

¹³C NMR (126 MHz, MeOD): δ=109.99 (C-1″), 100.09 (C-1′), 84.92 (C-5), 84.21 (C-4), 81.48 (C-3″), 75.33 (C-3), 75.19 (C-6′), 75.06 (C-5′), 73.17 (C-3′), 72.08 (C-4′), 71.20 (C-4″), 66.21 (C-2″), 55.94 (C-2′), 54.03 (C-5″), 50.58 (C-1), 48.16 (C-6), 42.93 (NHSO₂—CH₃), 36.49 (C-2), 15.03 (CH₃—C-6′).

MALDI TOFMS: C₁₉H₃₈N₄O₁₂S ([M+H]⁺) m/e 546.59; measured m/e 547.2.

### Preparation of 6′-(R)-Methyl-5-O-(5″-amino-5″-deoxy-β-D-ribofuranose)-2′,3-amino-1-N-benzamide paromamine (NB74-N1Bz; FIGS. 2 and 5)

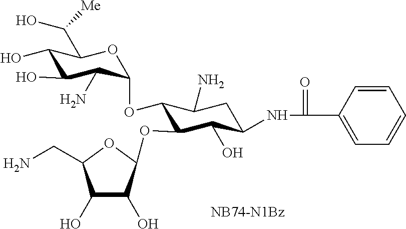

Compound NB74-N1Bz was prepared as described for the preparation of Compound NB74-N1PhS using Compound 22 (0.25 gram, 0.38 mmol), THF (5 ml), NaOH (0.1 M, 1.5 ml), and PMe₃ (1M solution in THF 2 ml), affording 114 mg (52%).

¹H NMR (500 MHz, MeOD) Ring I: δ=5.26 (d, 1H, J=2.8 Hz, H-1), 4.16 (dd,1H, J=7.0, 2.0 Hz, H-6), 3.87 (dd, 1H, J=9.9, 3.0 Hz, H-5), 3.60 (t, 1H, J=9.5 Hz, H-3), 3.30-3.23 (m, 1H, H-4), 2.67 (dd, 1H, J=10.3, 4.7 Hz, H-2), 1.23 (d, 1H, J=5.1 Hz, CHs-C-6). Ring II: δ=4.10-4.02 (m, 1H, 3-H), 3.63 (ddd, 2H, J=5.9, 2.0, 1.0 Hz, H-4, H-5), 3.47-3.41 (m, 1H, H-6), 2.97-2.88 (m, 1H, H-1), 2.09 (ddd, 1H, J=3.8, 2.5, 0.7 Hz, H-2 eq), 1.43 (dt, 1H,J=26.9, 6.7 Hz, H-2 ax). Ring III: δ=5.32 (d, 1H, J=2.0 Hz, H-1), 4.14 (dd, 1H, J=5.3, 2.1 Hz, H-2), 4.00-3.96 (m, 1H, H-3), 3.93 (dd, 1H,J=5.4, 3.8 Hz, H-4), 3.01 (dd, 1H, J=13.4, 4.0 Hz, H-5), 2.84 (dd, 1H, J=12.8, 7.7 Hz, H-5). Additional peaks in the spectrum were identified as follow:

δ=7.84 (d, 2H, J=7.4 Hz, Ph), 7.52 (t, 1H, J=7.4 Hz, Ph), 7.44 (t, 2H, J=7.5 Hz, Ph).

¹³CNMR (126MHz, MeOD): δ=168.98 (Amide), 134.34 (Ph), 131.24 (Ph), 128.07 (Ph), 127.02 (Ph), 109.66 (C-1″), 100.42 (C-1′), 84.93 (C-4), 84.66 (C-6), 82.41 (C-4″), 75.39 (C-5′), 75.04 (C-2″), 74.60 (C-4), 73.65 (C-3′), 72.19 (C-4′), 71.26 (C-3″), 66.35 (C-6′), 56.26 (C-2′), 50.93 (C-1), 50.36 (C-3), 43.50 (C-5″), 34.53 (C-2), 15.18 (CH₃—C-6′).

MALDI TOFMS: C₂₅H₄₀N₄O₁₁ ([M+H]⁺) m/e 572.27; measured m/e 573.22.

### Preparation of 6′-(R)-Methyl-5-O-[5″-(S)-amino-5″-deoxy-11-D-ribofuranose]-2′,3-amino-1-N-benzamide paromamine (NB124-N1MeS; FIGS. 2 and 5)

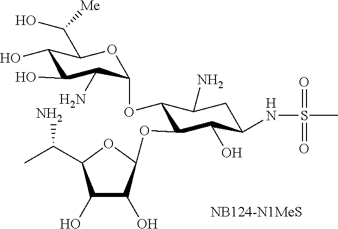

Compound NB124-N1MeS was prepared as described for the preparation of Compound NB74-N1PhS using: Compound 23 (0.230 gram, 0.358 mmol), THF (5 ml), aqueous NaOH (0.1 M, 2 ml), and PMe₃ (1M solution in THF 2 ml), affording 117mg (58%).

¹HNMR (500 MHz, MeOD): Ring I: δ=5.22 (d, 1H, J=3.2 Hz, H-1), 4.16-4.04 (m, 1H, H-4), 3.81 (dd, 1H, J=9.9, 3.1 Hz, 1HH-5), 3.53-3.48 (m, 1H, H-3), 3.21 (dd, 1H, J=10.2, 8.7 Hz, H-6), 2.62 (dd, 1H, J=10.4, 4.4 Hz, H-2), 1.20 (d, 3H, J=5.8 Hz, CH₃—C-6). Ring II: δ=3.54 (t, 1H, J=9.1 Hz, H-5), 3.35 (dt, 2H, J=13.4, 9.3 Hz, H-4, H-6), 3.28-3.20 (m, 1H, H-1), 2.86-2.77 (m, 1H, H-3), 2.11 (dd, 1H, J=8.5, 4.1 Hz, H-2 eq), 1.33 (dd, 1H, J=26.2, 12.4 Hz, H-2 ax). Ring III: δ=5.26 (d, 1H, J=2.6 Hz, H-1), 4.08 (dd, 1H, J=5.4, 2.4 Hz, H-2), 4.04-3.98 (m, 1H, H-3), 3.59 (dd, 1H, J=7.3, 6.3 Hz, H-4), 2.99 (t, 1H, J=7.1 Hz, H-5), 1.20 (d, 3H, J=7.1 Hz, CH₃—C-5′). Additional peaks in the spectrum were identified as follow: δ=3.03 (s, 3H, NHSO₂—CH₃).

¹³CNMR (126 MHz, MeOD): δ=108.75 (C-1″), 100.26 (C-1′), 86.48 (C-4″), 84.55 (C-4), 83.75 (C-5), 75.31 (C-6), 74.99 (C-2″), 74.97 (C-5′), 73.88 (C-3′), 72.15 (C-6′), 71.07 (C-3″), 66.36 (C-4′), 56.22 (C-2′), 54.15 (C-1), 50.65 (C-3), 49.56 (C-5″), 40.39 (NHSO₂—CH₃), 36.69 (C-2), 17.29 (CH₃—C-5′), 15.14 (CH₃—C-6′).

MALDI TOFMS: C₂₀H₄₀N₄O₁₂S ([M+H₂O]⁺) m/e 560.24; measured m/e 561.2.

### Preparation of 6′-(R)-Methyl-5-O-(5-(R)-amino-5-deoxy-β-D-ribofuranose)-2′,3-amino-1-N-acetamide paromamine (NB124-N1Ac; FIGS. 2 and 5)

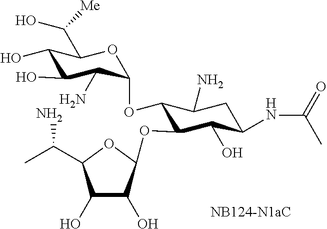

Compound NB124-N1Ac was prepared as described for the preparation of Compound NB74-N1PhS using Compound 24 (0.820 gram, 1.36 mmol), THF (10 ml), aqueous NaOH (0.1 M, 2 ml), and PMe₃ (1M solution in THF 2 ml), affording 370 mg (52%).

¹H NMR (500 MHz, MeOD): Ring I: δ=5.21 (d,1H, J=4.8 Hz, H-1), 4.07 (qd, 1H, J=6.4, 2.8 Hz, H-6), 3.77 (d,1H, J=12.1 Hz, H-5), 3.47 (dd,1H, J=10.4, 8.9 Hz, H-3), 3.15 (t, 1H, J=9.4 Hz, H-4), 2.60 (dd, 1H, J=10.0, 3.2 Hz, H-2), 1.14 (d, 3H, J=6.7 Hz, CH₃—C-6). Ring II: δ=3.71 (td, 1-H, J=12.3, 4.1 Hz, H-5), 3.58-3.50 (m, 1H, H-6), 3.36 (dt, 2H, J=19.3, 9.4 Hz, H-1, H-4), 2.82 (ddd, 1H, J=13.3, 9.7, 4.0 Hz, H-3), 1.93 (dt, 1H, J=7.4, 4.0 Hz, H-2 eq), 1.23 (q, 1H, J=12.5 Hz, H-2 ax). Ring III: δ=5.22 (s, 1H, H-1), 4.06 (dd, 1H, J=4.3, 2.3 Hz, H-2), 4.00-3.95 (m, 1H, H-3), 3.64 (dd,1H, J=8.9, 5.8 Hz,H-4), 3.11 (dd, 1H, J=7.5, 6.4 Hz,H-5), 1.24 (d, 3H, J=8.0 Hz, CH₃—C-5′).

¹³CNMR (126 MHz, MeOD): δ=172.08 (Amide), 109.46 (C-1″), 99.80 (C-1′), 85.02 (C-4), 84.22 (C-4″), 83.10 (C-3′), 75.41 (C-6′), 75.36 (C-5′), 74.93 (C-1), 73.27 (C-6), 72.01 (C-3″), 71.75 (C-5″), 66.09 (C-2″), 55.70 (C-2′), 50.65 (C-3), 50.29 (C-4′), 49.77 (C-5), 34.16 (C-2), 21.37 (Ac), 15.49 (CH₃—C-5″), 14.93 (CH₃—C-6′).

MALDI TOFMS: C₂₁H₄₀N₄O₁₁([M+H]⁺) m/e 524.56; measured m/e 525.26.

### Preparation of 6′-(R)-Methyl-5-O-[5″-(S)-amino-5″-deoxy-β-D-ribofuranose]-2′,3-amino-1-N-benzamide paromamine (NB124-N1Bz; FIGS. 2 and 5)

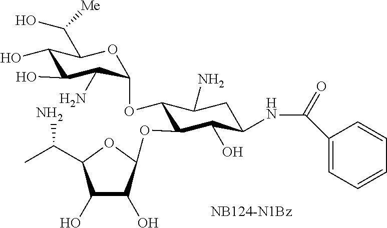

Compound NB124-N1Bz was prepared as described for the preparation of Compound NB74-N1PhS, using Compound 25 (0.260 gram, 0.390 mmol), THF (5 ml), aqueous NaOH (0.1 M, 2 ml), and PMe₃ (1M solution in THF 2 ml), affording 160 mg (70%).

¹NMR (500 MHz, MeOD): Ring I: δ=5.22 (d, 1H, J=3.1 Hz, H-1), 4.09 (dd, 1H, J=19.0, 8.6 Hz, H-3), 3.82 (dd, 1H, J=10.0, 3.0 Hz, H-5), 3.53-3.47 (m, 1H, H-4), 3.19 (dd, 1H, J=10.1, 8.8 Hz, H-6), 2.60 (dd, 1H, J=11.1, 5.1 Hz, H-2), 1.18 (d, 3H, J=5.3 Hz, CHs-C-6). Ring II: δ=4.05-3.95 (m, 1H, H-1), 3.57 (dd, 2H, J=7.6, 4.3 Hz, H-6, H-5), 3.39 (dd, 1H, J=12.6, 6.1 Hz, H-4), 2.92-2.82 (m, 1H, H-3), 2.03 (dd, 1H, J=8.4, 4.0 Hz, H-2 eq), 1.37 (dd, 1H, J=25.9, 12.5 Hz, H-2 ax). Ring III: δ=5.25 (d,1H, J=2.3 Hz, H-1), 4.07 (dd, 1H, J=5.4, 2.2 Hz, H-2), 4.00-3.95 (m, 1H, H-3), 3.58-3.53 (m, 1H, H-4), 2.98-2.91 (m, 1H, H-5), 1.16 (d, 3H, J=7.2 Hz, CH₃—C-5′). Additional peaks in the spectrum were identified as follow: δ=7.81 (d,2H, J=7.3 Hz, Ar), 7.50 (t, 1H, J=7.3 Hz, Ar), 7.43 (t, 2H, J=7.5 Hz, Ar).

¹³C NMR (126 MHz, MeOD): δ=168.99 (Carbonyl), 134.36 (Ar), 131.22 (Ar), 128.05 (Ar), 126.98 (Ar), 108.81 (C-1″), 100.33 (C-1′), 86.56 (C-4″), 84.92 (C-4), 83.87 (C-6), 75.36 (C-5′), 75.05 (C-2″), 74.46 (C-5), 73.96 (C-4′), 72.14 (C-6′), 71.14 (C-3″), 66.30 (C-3′), 56.30 (C-2′), 50.91 (C-3), 50.36 (C-1), 49.61 (C-5″), 34.59 (C-2), 17.34 (CHs-C-5′), 15.06 (CH₃—C-6′).

MALDI TOFMS: C₂₆H₄₂N₄O₁₁ ([M+H]⁺) m/e 586.63; measured m/e 587.29.

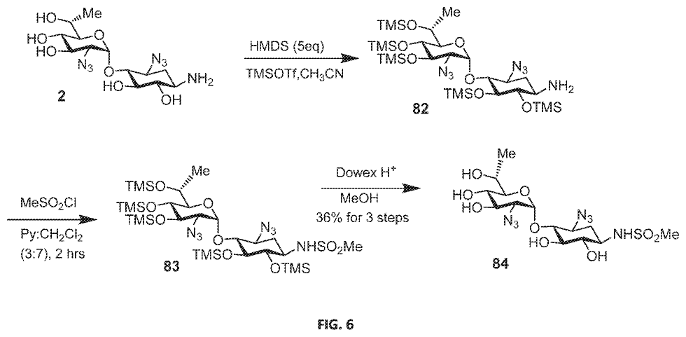

An alternative synthetic pathway for preparing N1-sulfonyl modified pseudo-disaccharide compounds which can be further utilized as Acceptor compounds for preparing Set1 and Set2 compounds (upon introducing hydroxy-protecting groups in respective positions as described herein), denoted herein Acceptors B*, from Intermediate A (see, FIG. 3) or Compound 2 (see, FIG. 4) is depicted in FIG. 6. The total yield is 36%. The structures of intermediate compounds 82 and 83, and of the obtained Compound 84 are verified using NMR and MS measurements as described herein.

### Example 2

### Activity and Toxicity of the Compounds of Example 1

All the tested aminoglycosides were used in their sulfate salt forms. Molecular Weights (grams/mol) of the sulfate salts are as follow:

NB74-N1PhS—726.40, NB74-N1Ac—581.62, NB74-N1MeS—608.74, NB74-N1Bz—634.23, NB124-N1MeS—640.76, NB124-N1Ac—559.28, NB124-N1Bz—667.75.

Readthrough Activity; In Vitro Studies:

The Set1 and Set2 compounds as described in Example 1 hereinabove were tested for their readthrough activity in vitro by using a dual luciferase reporter assay system, as follows.

DNA fragments derived from PCDH15, CFTR, and IDUA cDNAs, including the tested nonsense mutation or the corresponding WT codon, and four to six upstream and downstream flanking codons, were created by annealing the following pairs of complementary oligonucleotides:

Usher Syndrome (PCDH15):
p.R3Xmut/wt:
5′-GATCCCAGAAGATGTTTT/CGACAGTTTTATCTCTGGAC
AGAGCT-3′;
and
5′-CTGTCAGAGATAAAACTGTCA/GAAACATCTTCTG-3′.
p.R245Xmut/wt:
5′-GATCCAAAATCTGAATGAGAGGT/CGAACCACCACCACC
ACCCTCGA-GCT-3′;
and
5′-CGAGGGTGGTGGTGGTTGTTCG/ACCTCTCATTCAGATT
TTG-3′.
Cystic Fibrosis (CFTR):
p.G542Xmut/wt:
5′-TCGACCAATATAGTTCTTT/GGAGAAGGTGGAATCGAGC
T-3′;
and
5′-CGATTCCACCTTCTCA/GAAGAACTATATTGG-3′.

Fragments were inserted in frame into the polylinker of the p2Luc plasmid between either BamHI and Sad (p.R₃X and p.R₂₄₅X) or Salland Sad (all the rest) restriction sites. For the in vitro readthrough assays, the obtained plasmids, with addition of the tested aminoglycosides were transcribed and translated using the TNT Reticulocyte Lysate Quick Coupled Transcription/Translation System. Luciferase activity was determined after 90 minutes of incubation at 30° C., using the Dual Luciferase Reporter Assay System (Promega™). Stop codon readthrough was calculated as described in Grentzmann et al. RNA 4, 479-486 (1998).

Reporters carrying R₃X nonsense mutation (a premature UGA C stop codon) related to Usher syndrome and G542X (a premature UGA G stop codon) related to Cystic fibrosis were used, and the obtained data is presented in FIGS. 7A-D.

As can be seen in FIGS. 7A-D, all the tested aminoglycosides, except NB74-Bz and NB124-Bz (data not shown) induced readthrough. Of the aminoglycosides tested, NB124-MeS induced the highest level of readthrough, at all tested concentrations, followed by NB74-MeS. For the R₃X nonsense mutation, NB74-MeS exhibited about 1.5 folds better readthrough activity in comparison to NB74; and NB124-MeS also exhibited readthrough activity better than NB124. For the G542X mutation, compounds NB74-MeS and NB124-MeS exhibit better activity in comparison to NB74 and NB124, respectively, confirming the substantial influence of the methane sulfonyl moiety.

The obtained data suggests that a methane sulfonyl substitution at the N1 position contributes to the interactions of NB compounds with the eukaryotic A site and induces their readthrough potency.

Protein Translation Inhibition Tests:

The Set1 and Set2 compounds as described in Example 1 hereinabove were further tested for their ability to inhibit eukaryotic translation, as follows.

Eukaryotic in vitro translation inhibition was quantified using a TNT® T7 Quick Coupled

Transcription/Translation System with a luciferase T7 control DNA plasmid (Promega), according to the manufacturer protocol. Translation reactions (25 μL) containing variable concentrations of the tested aminoglycoside were incubated at 30° C. for 60 minutes, cooled on ice for 5 minutes, diluted with the dilution reagent and transferred into 96-well plates. In both prokaryotic and eukaryotic systems the luminescence was measured immediately after the addition of the Luciferase Assay Reagent (50pL; Promega), and the light emission was recorded with a FLx800 Fluorescence Microplate Reader (Biotek). The half-maximal inhibition concentration (IC₅₀) values were obtained from fitting concentration-response curves to the data of at least two independent experiments by using Grafit 5 software.

The obtained data is presented in Table 1.

TABLE 1

| Compound | IC₅₀^{Euk} (μM) |
|---|---|
| NB74 | 14.31 ± 0.93 |
| NB74-Bz | 754.77 ± 780.84 |
| NB74-MeS | 8.35 ± 0.41 |
| NB74-Ac | 92.3 ± 9.9 |
| NB74-PhS | 160.4 ± 3.6 |

As shown in Table 1, comparing the IC₅₀ values of NB74-MeS (IC₅₀=8.35 μM) and its parent structure NB74 (IC₅₀=14.31 μM) indicates that NB74-MeS is 1.7-folds more specific to the eukaryotic ribosome, while NB74-Bz (IC₅₀=754.77 μM) is 54 folds less specific to the eukaryotic ribosome than NB74. This collective data suggest that the observed impact of methyl sulfonyl group on the elevated readthrough activity of NB74-MeS is associated with its increased specificity to the eukaryotic ribosome.

Readthrough Activity in Cell-Free Assay:

A dual luciferase plasmid carrying the Cystic fibrosis transmembrane conductance regulator (CFTR) mutation, S466X, or wild-type sequence, between Renilla and Firefly luciferase was transcribed and translated in vitro using rabbit reticulocytes (TNT mix) and then tested for functional activity of firefly and renilla luciferases. WT plasmids expressed both firefly and renilla luciferases while mutant plasmids expressed only the renilla luciferase due to the stop codon found in the inserted sequence. The readthrough assays were conducted for the tested compounds and controls by adding the compounds to the in vitro transcription/translation reaction mixture.

Cystic Fibrosis (CFTR):
p.S466Xmut/wt:
5′-GGCAAGACTTGACTTCTAATGGTG-3′;
and
5′-GGCAAGACTTCACTTCTAATGGTG-3′.

Read-through activity induced by NB74-MeS and NB124-MeS was calculated from the dual luciferase assay data using results from both the WT and mutant plasmids and the following equation:

$$\% Readthrough = \frac{Luminiscence ( {FF}_{mut} ) / Luminiscence ( {Renilla}_{mut} )}{Luminiscence ( {FF}_{wt} ) / Luminiscence ( {Renilla}_{wt} )}$$

The following parameters were also calculated from the dual-luciferase assay results for both the WT and mutant plasmids:

(i) Translational Inhibition (TI; The extent of the translational inhibition of ribosomes by NB74-MeS and NB124-MeS was calculated using the reduction in the Renilla bioluminescence values of WT plasmid by nonlinear regression curve fit on a plot of Relative Light Units (RLU) versus the log NB74-MeS and NB124-MeS molar concentration (Find ECanything analysis, GraphPad PRISM, version 7).

(ii) NB74-MeS and NB124-MeS readthrough potency (EC50, μM Readthrough activity of NB74-MeS and NB124-MeS was presented as Firefly luciferase activity normalized to Renilla luciferase activity in mutant plasmids. Normalized Firefly values were plotted against the logarithmic molar NB74-MeS and NB124-MeS concentrations and the curve was fitted using four parameters nonlinear regression (GraphPad PRISM software, Version 7). NB74-MeS and NB124-MeS at high concentration causes translation inhibition, which does not allow further dose escalation. Therefore, to allow EC50 calculation, the range of values was taken up to the NB74-MeS and NB124-MeS concentration that was smaller than the TI80 (determined above).

(iii) Readthrough efficiency is presented as fold-increase in Firefly luciferase activity between NB74-MeS and NB124-MeS treated and non-treated mutant plasmid. Fold increase was calculated at NB74-MeS and NB124-MeS concentration which showed <20% inhibition at steady state Renilla activity on wild-type control plasmid.

(iv) % readthrough of WT is calculated as the percentage of FF/Renilla of the mutant plasmid divided by the FF/Renilla of the WT plasmid.

Incubation of NB74-MeS and NB124-MeS with cystic fibrosis S466X nonsense mutation resulted with dose-response suppression in the cell-free assay (data not shown). The calculated TI80 values were 16 μM for NB74-MeS and 1 μM for NB124-MeS. The calculated TI50 values were>16 μM for NB74-MeS and 2 μM for NB124-MeS.

Table 2 below presents the results of cystic fibrosis S466X nonsense mutation suppression dose-response cell-free assays conducted for the two exemplary compounds according to embodiments of the present invention, NB74-MeS and NB124-MeS, at a concentration range of 0-16 μM. The data presented in Table 2 demonstrate that incubation of NB74-MeS and NB124-MeS at escalating doses resulted in substantial increase (from about 2-folds to about 10-folds) in read-through compared with the control (non-treated cell extracts) and 9-17% read-through compared with wild-type control plasmid.

TABLE 2

| | Fold Read- | % Read- | | |
|---|---|---|---|---|
| | through at | through at | | |
| Compound | TI80* | TI80** | TI80 (μM) | TI50 (μM) |
| NB74-MeS | 9.9 | 17 | 16 | >16 |
| NB124-MeS | 2.4 | 9 | 1 | 2 |

*relative to MUT control
**relative to WT

These data further support the findings that inclusion of a sulfonyl moiety at the N1 position has a pronounced effect on the biological activity of aminoglycosides.

Toxicity Assays in Cochlear Explants:

The ototoxicity potential of Set1 and Set2 compounds was tested with cochlear explants as described, for example, in E. Shulman et al., J. Biol. Chem. 289(4), 2318-2330 (2014). The ototoxicity was measured as the value of AG concentration that is needed for 50% of hair loss from the outer hair cells of the cochlea, as follows.

The tested compounds were screened for toxicity to auditory hair cells in explants of the organ of Corti from CBA/J mice of postnatal day 2-3. The dissected tissue was placed on a collagen-coated incubation dish as a flat surface preparation in 1 ml of serum-free Basal Medium Eagle (Sigma Aldrich, St. Louis, Mo.) plus serum-free supplement (Invitrogen, Eugene, Oreg.), 1% bovine serum albumin, 2 mM glutamine, 5 mg glucose /ml and 10 units penicillin/ml. The explants were incubated for 5 hours (37° C., 5% CO₂) and an additional 1 ml of culture medium was added to submerse the explants. After 48 hours of continued pre-incubation the culture medium was exchanged for new medium containing a specified concentration of the tested compound. Following another 72 hours of incubation, the explants were washed three times with PBS, fixed overnight in 4% (v/v) paraformaldehyde at 4° C., and then permeabilized for 30 minutes with 0.3% (v/v) Triton X-100 in PBS. The specimens were washed three times with PBS for 10 minutes at room temperature and incubated with rhodamine phalloidin (1:100; Life Technologies, Eugene, OR), either for 1 hour at room temperature or overnight at 4° C.

Following several rinses with PBS, the explants were mounted on microscope slides with Fluoro-Gel (Electron Microscopy Sciences, Hatfield, Pa.) and imaged on a Leica SP5 Confocal TCS Microscope (Leica, Wetzlar, Germany). Hair cells were identified by phalloidin-staining of their stereociliary bundles and circumferential F-actin rings. Their presence or absence was quantified using a 50x oil immersion objective on an epifluorescence Leitz Orthoplan microscope (Leica, Wetzlar, Germany), equipped with a calibrated scale (0.19 mm) superimposed on the field of view. All rows of hair cells were oriented longitudinally within each frame and counted from the apex of the cochlea to its base. Cell counts were entered in a computer program and compared to a normative database (KHRI Cytocochleogram, version 3.0.6, Kresge Hearing Research Institute, University of Michigan, Ann Arbor, Mich., USA) and are reported as percent hair cell loss averaged over the entire length of the explant.

FIG. 8 and Table 3 below present the obtained comparative dose-response curves and show that 50% loss of hair cells (LC₅₀^{Coch}) was observed at concentrations of 19 μM for NB124-MeS and 61.77 μM for NB74-MeS. The value obtained for NB124-MeS is favorable to NB124 (LC₅₀^{Coch}=11.1 μM). The ototoxicity of NB124-MeS is almost two-folds lower than that of NB124.

NB74-Ac and NB124-Ac were both found to exhibit incredibly low ototoxicity potential (LC₅₀^{Coch} values of both are in the range of millimolar and not micromolar). For example, in the case of acetate substitution of NB74, the ototoxicity has decreased in three orders of magnitude (NB74-Ac LC₅₀^{Coch}=2.4 mM; NB74 LC₅₀^{Coch}=112 μM).

These data also show that for NB74-PhS, NB74-Ac and NB124-Ac, the ototoxicity data is in direct correlation with the readthrough activity and the inhibition of the eukaryotic translation data. The cochleotoxicity of these compounds is significantly decreased due to their low affinity for the eukaryotic ribosome.

TABLE 3

| | Ototoxicity | |
|---|---|---|
| Compound | (Loss of outer hair cells) | |
| NB74 | 112 | μM |
| NB74-Ac | 2.04 | mM |
| NB74-MeS | 61.77 | μM |
| NB74-PhS | 176 | μM |
| NB124 | 11.1 | μM |
| NB124-Ac | 1.6 | mM |
| NB124-MeS | 19 | μM |

Overall, these data suggest that there should be a subtle balance between the electronic and stearic interactions of the substituent with the target MET channels that influence on the cochleoatoxicity. Nevertheless, an observed increased readthrough activity along with a reduced cochleotoxicty demonstrated for NB124-MeS is substantial and suggests that it is a promising drug candidate.

The comparative ototoxicity of NB124-MeS, NB124-Ac and NB124 was further tested by measuring the cochlear cell loss, and the obtained data is presented in FIGS. 9A-D. As shown therein, staining for F-actin revealed organized outline of the array of three rows of outer hair cells (OHC) and one row of inner hair cells (IHC) in segments from the basal part of the cochlea (see, FIG. 9A). In the presence of 15 μM of NB124-MeS the structure of the epithelium remained normal and almost indistinguishable from the control with indication of average damage of 10% (see, FIG. 9D). In contrast, hair cell loss was almost complete in the presence of 15 μM of NB124 indicating over 80-90% cell loss (see, FIG. 9B). For NB124-Ac at concentration of 150 μM the epithelium remained normal and indistinguishable from the control (see, FIG. 9C). The data obtained in these studies demonstrate that substitution at the N1 position with methane sulfonyl group has a significant contribution to the readthrough activity of the compounds. In addition, NB124-MeS has showed a significant decrease in its ototoxicity level in comparison to its parent NB124.

### Example 3

### Exemplary 6′-Modified Compounds According to Some of the Present Embodiments

The present inventors have performed some modeling studies, based on the recently solved structures of G418 bound to leishmania A-site rRNA sequence (Shalev et al., PNAS 110, 13333-338, (2013), and the structure of G418 bound to the yeast ribosome (M. Yusopov et al., Nature 513, 517-22 (2014). These studies suggested that conversion of C6′-OH of ring I to the corresponding carboxylic acid or amide (C(=O)OH and/or C(═O)NH₂) could bring Ring I of the aminoglycosides closer to the G1408 in the mammalian ribosome by forming strong electrostatic and or H-bond interaction between the carboxylate/amide and the G1408 residue. This hypothesis was further supported by experimental data that showed that compounds with 6′-NH₂ (like neomycin and gentamicin) are inactive against leishmania parasite and their activity/binding to the mammalian ribosome are significantly poorer than compounds with 6′-OH (e.g. paromomycin and G418) (Shalev et al., PNAS 110, 13333-338, (2013), which was explained by electrostatic repulsion between the 6′-NH₃+ (ammonium) and the G1408; and by experimental data showing that such pseudo-disaccharide compounds, which feature C(═O)OH or C(═O)NH₂ at the C6′ position were previously synthesized (Simonson et al., ChemBioChem 3, 1223-28, 2002) and were found to lack any antibacterial activity, with IC50 values against prokaryotic ribosome which are about two orders of magnitude poorer than that of the parent paromamine (6′-OH).

While such compounds have not been studied in the context of mammalian ribosome, the present inventors have designed and synthesized pseudo di- and tri-saccharide compounds, as depicted in FIG. 10, and have studied their interaction with the G1408 residue in the mammalian cytoplasmic A-site.

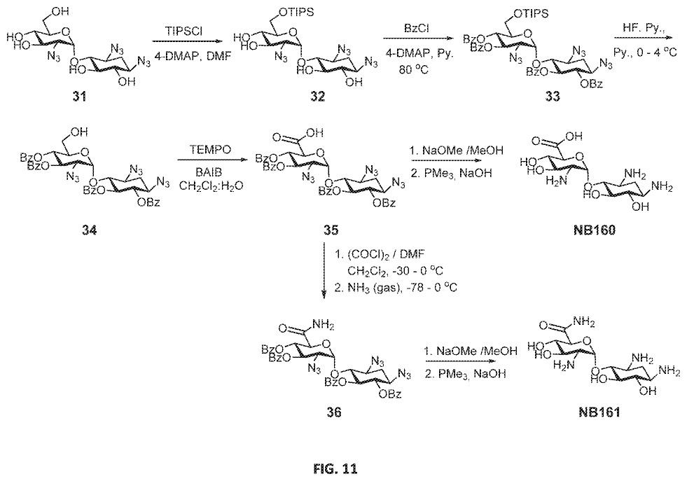

FIG. 11 presents the general synthetic pathway of pseudo di-saccharide aminoglycosides NB160 and NB161, depicted in FIG. 10.

FIG. 12 below presents the general synthetic pathway of the newly designed pseudo tri-saccharide aminoglycosides NB162-165, depicted in FIG. 10.

Preliminary readthrough activity tests of the compounds depicted in FIG. 10 on R3X mutation, performed as described in Example 2 hereinabove, showed that these compounds exhibit a low readthrough activity (data not shown).

This was corroborated also by eukaryotic translation system assays performed as described in Example 2 hereinabove. The measured IC₅₀ values of the tested compounds against mammalian translation system showed poor inhibition (data not shown).

In a series of modeling, docking and molecular simulations studies it was found that in compounds featuring a carboxylic acid at the C6′ position there is an internal salt bridge formation between the carboxylate and the N5″-ammonium, which significantly shifts the binding interaction of the ligands with the RNA host.

### Example 4

### Exemplary 4′-Modified Compounds According to Some of the Present Embodiments

In continuous attempts towards new structural design of aminoglycosides towards eliminating their ototoxic effects, while preserving their potent readthrough activity, the present inventors have considered recent reports of reduced ototoxicity of paromomycin derivatives which were modified either at the O4′ or at the O4′ and O6′ positions (see, Perez-Fernandez, D. et al. at. Commun. 5, 3112 (2014); Akbergenov, R. et al. Am. Soc. Microbiol. 5, 1-10 (2014)). It is noted that similar 4′-O-alkylations on kanamycin did not result in increased ribosomal selectivity and reduced toxicity as in the case of paromomycin (see, Kato, T. et al. ACS Infect. Dis. 1, 479-486 (2016)). The present inventors have designed a series of compounds, based on the previously described NB74 and NB124 pseudo tri-aminoglycosides, which feature modifications at the O4′ position (also referred to herein as Set4 compounds) or at the O4′ and O6′ positions (also referred to herein as Set3 compounds), as depicted in FIG. 13, each optionally in combination with N1-substitutions (e.g., N1-methylsulfonyl substitution) as described in Example 1 hereinabove, and as depicted in FIG. 14 (also referred to herein as Set5 and Set6 compounds).

As illustrated in FIG. 13, the newly designed structures include, for example, the modification of the parent NB74 and NB124 either with 4′,6′-ethylidine (Compounds 61 and 63), or with 4′,6′-benzylidine (Compounds 62 and 64), in Set3, or with 4′-O-ethyl (Compounds 65 and 67), or 4′-O-propyl (Compounds 66 and 68), in Set4.

FIG. 15 presents a general synthetic pathway for Set3 Compounds.

Briefly, for the synthesis of the Set3 structures, the commercial G418 is first converted to Intermediate A (see, FIG. 3) in two chemical steps (acid hydrolysis and perazidation) according to the chemical steps as described in Nudelman et al. 2010, supra. The cyclic acetal formation at 4′,6′-hydrozyls is followed by selective acetylation to afford Acceptors C. Coupling reactions with previously described trichloroacetamide donors (Nudelman et al. 2006, supra) followed by two deprotection steps afford the Set3 structures.

Exemplary processes of preparing exemplary Acceptors C, in which Rw is Ph (Compound 72) or para-methoxyphenyl (PMP; Compound 74), are presented in FIG. 16, and are described in the following. Briefly, the common intermediate 2 was obtained from the commercial G418 in two chemical steps (acid hydrolysis and perazidation) as previously described [Nudelman et al. Bioorganic Med. Chem. 18, 3735-3746 (2010)]. The intermediate 2 was then subjected to cyclic acetal formation at 4′,6′-hydrozyls by selective acetylation with benzaldehyde dimethyl acetal, or with p-methoxybenzaldehyde dimethyl acetal to afford either the selectively protected 71 or 73, respectively. These two acetals were then separately converted to the corresponding common acceptors, 72 and 74, by reaction with acetic anhydride at low temperature.

Donors 75 and 76 (shown in the inset of FIG. 16) were prepared as previously described [Joseph et al. Chem. Commun. 51, 104-106 (2015)].

Coupling of each of acceptors 72 and 74 with the donor 75 or donor 76 is then effected using previously described procedures.

A similar sequence of steps is performed for the assembly of the Set4 structures, as presented in FIG. 17.

Briefly, Intermediate A is first converted to Intermediate D by using similar sequence of steps as previously reported for paromomycin (Perez-Fernandez, D. et al. at. Nature Commun. 5, 3112 (2014); Akbergenov, R. et al. Am. Soc. Microbiol. 5, 1-10 (2014)). Intermediate D is then converted to the common Acceptors E by various alkylations of the C4′ position and selective removal of PMB protection either with CAN or DDQ, followed by acetylation to afford the desired acceptors that contain C5-OH free for the attachment of the ring III. The coupling of trichloroacetimidate donors and two deprotection steps then affords the Set4 compounds.

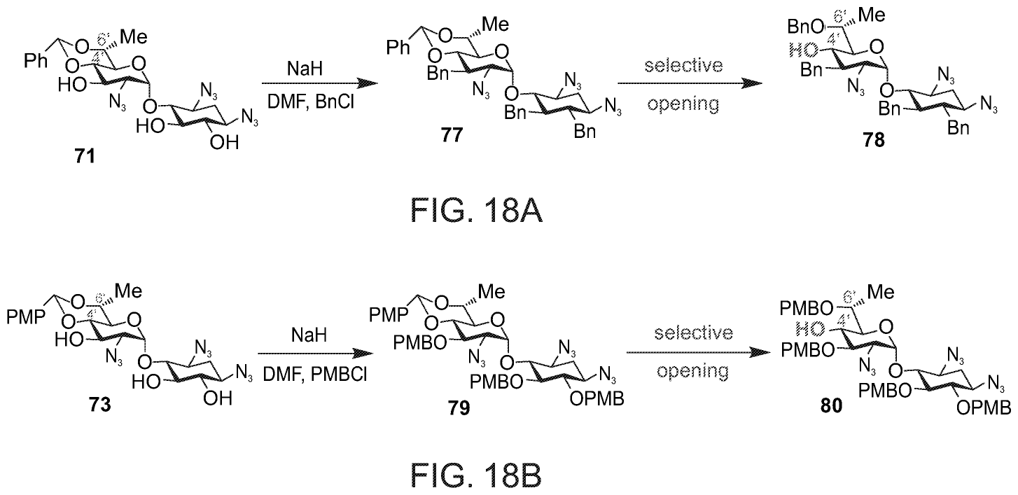

An alternative synthetic pathway is presented in FIGS. 18A and 18B. Briefly, cyclic acetals 71 and 73 (see, FIG. 16, respectively) were first converted to the completely protected intermediate acetals 77 and 79, respectively, and these are then subjected to selective opening of the cyclic benzylidene acetal to afford Compounds 78 and 80, respectively.

In yet another alternative procedure, the cyclic acetals in 71 and 73 are removed to yield the corresponding 4′,6′-diols, which are then subjected to selective protection of the exocyclic 6′-OH with a bulky protecting group that is stable under basic conditions required to introduce an alkyl group at the 4′ position.

FIG. 14 presents chemical structures combining the structural modifications introduced in Set1 and Set2 (e.g., N1-SMe modification) as described herein with the structural modifications shown in Set3 and Set4 as described herein, to afford compounds collectively referred to herein as Set5 and Set6 structures.

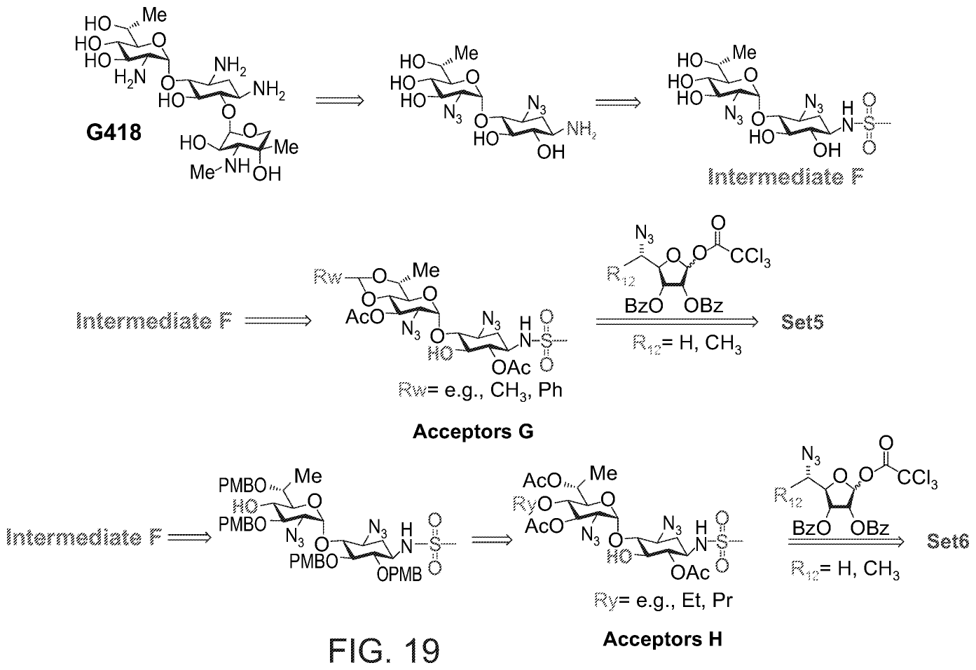

FIG. 19 presents a general synthetic pathway for the preparation of Compounds of Set5 and Set6.

Briefly, the commercially available G418 is first converted to Intermediate F, which contains the methane sulfonyl substitution on N1 position of ring II, as described in Example 1 hereinabove. Next, the Intermediate F is further alkylated to produce common Acceptors G and/or H series, which are then subsequently coupled with trichloroacetimidate donors of the third ring, as previously described herein. The two steps deprotection then provides the Compounds of Set5 and Set6.

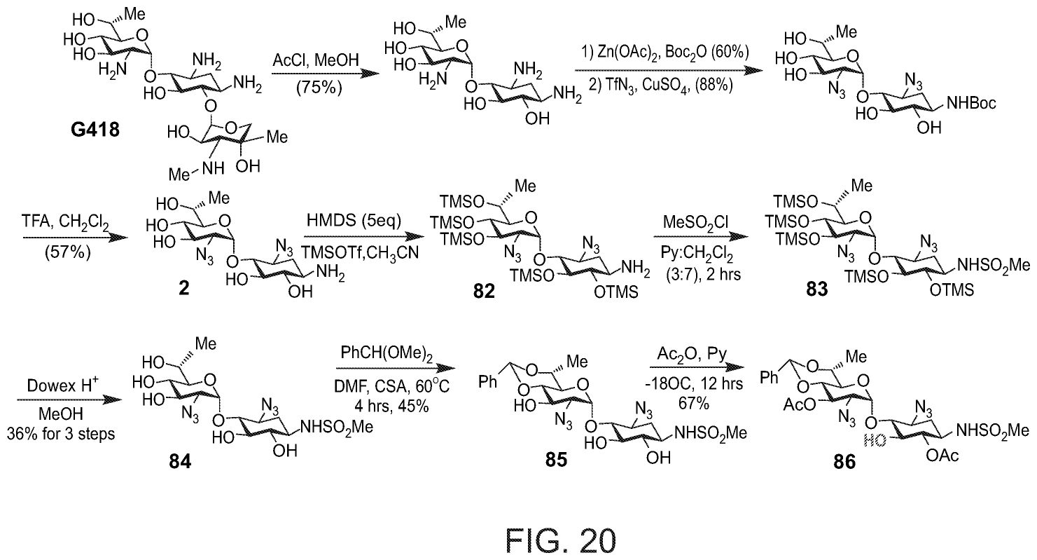

FIG. 20 presents an exemplary synthetic pathway for the preparation of an exemplary Acceptor G, Compound 86.

Briefly, the amine 2 was prepared and then converted to the corresponding N1-methanesolfonate 84, as described hereinabove and shown in FIG. 6. Benzylydene protection to afford Compound 85 was then followed by a selective protection of the alcohols to yield Compound 86 as an exemplary Acceptor G with good isolated yields. Acceptor H is similarly prepared from, for example, Compound 84. Compounds of Set3, Set4, Set5 and Set6 (stored as their sulfate salts as described in Example 1 hereinabove) are subjected to comparative readthrough activity assays, inhibition of translation assays and toxicity tests, as described in Example 2 hereinabove, using Compounds NB74 and NB124 as the parent, reference compounds. Compounds exhibiting good readthrough activity and low cytotoxicity are then subjected to cochleotoxicity tests as described in Example 2 hereinabove.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A compound represented by general formula I: 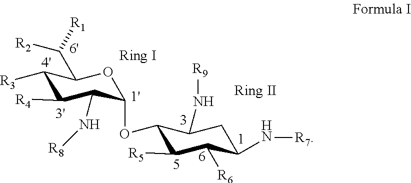
or a pharmaceutically acceptable salt thereof, wherein:
the dashed line indicates a stereo-configuration of position 6′ being an R configuration or an S configuration;
R₁ is selected from hydrogen, a substituted or unsubstituted alkyl, a substituted or unsubstituted cycloalkyl, and a substituted or unsubstituted aryl;
R₂ is selected from hydrogen, a substituted or unsubstituted alkyl and ORx, wherein Rx is selected from hydrogen, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl, or, alternatively, R₂ is said ORx and forms together with R₃ a dioxane;
R₃ is selected from hydrogen, a substituted or unsubstituted alkyl and ORy, wherein Ry is selected from hydrogen, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl, or, alternatively, R₃ is said ORy and formed together with R₂ a dioxane;
R₄—R₆ are each independently selected from hydrogen, a substituted or unsubstituted alkyl, and ORz, wherein Rz is selected from hydrogen, a monosaccharide moiety, an oligosaccharide moiety, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl; and
R₇—R₉ are each independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a sulfonyl,
provided that at least one of R₇—R₉ is a sulfonyl.

2. The compound of claim 1, wherein R₇ is said sulfonyl, the compound being represented by Formula Ia: 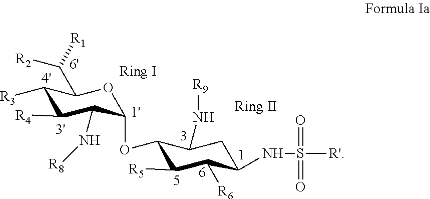
wherein:
R₁—R₆, R₈ and R₉ are as defined for Formula I; and
R′ is selected from hydrogen, a substituted or unsubstituted alkyl, a substituted or unsubstituted cycloalkyl, a substituted or unsubstituted alkaryl, and a substituted or unsubstituted aryl.

3. The compound of claim 2, wherein R′ is selected from an unsubstituted alkyl and an unsubstituted aryl.

4. The compound of claim 2, wherein R′ is methyl.

5. The compound of claim 2, wherein R₈ and R₉ are each hydrogen.

6. The compound of claim 2, wherein R₂ is ORx, and Rx is selected from hydrogen and a substituted or unsubstituted alkyl.

7. The compound claim 2, wherein R₃ is ORy, and Ry is selected from hydrogen and a substituted or unsubstituted alkyl.

8-16. (canceled)

17. The compound of claim 1, wherein each of R₄—R₆ is ORz.

18. (canceled)

19. (canceled)

20. The compound of claim 1, wherein R₅ is ORz and Rz is said monosaccharide moiety.

21. (canceled)

22. The compound of claim 1, wherein R₅ is ORz and Rz is said monosaccharide moiety represented by Formula II, the compound being represented by Formula III: 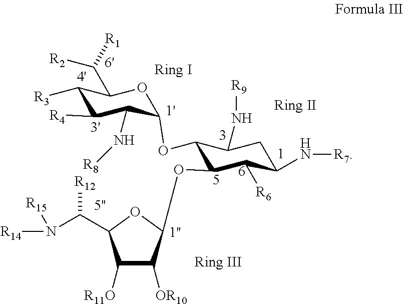
wherein:
R₁—R₄ and R₆—R₉ are each as defined for Formula I or Formula Ia; and
R₁₀ and R₁₁ are each independently selected from hydrogen, a substituted or unsubstituted alkyl, a substituted or unsubstituted cycloalkyl, a substituted or unsubstituted aryl, a substituted or unsubstituted alkaryl, and an acyl;
R₁₂ is selected from the group consisting of hydrogen, a substituted or unsubstituted alkyl, a substituted or unsubstituted cycloalkyl, and a substituted or unsubstituted aryl; and
each of R₁₄ and R₁₅ is independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl, a sulfonyl and a cell-permealizable group, or, alternatively, R₁₄ and R₁₅ form together a heterocyclic ring.

23. The compound of claim 22, wherein R₇ is said sulfonyl, the compound being represented by Formula IIIa: 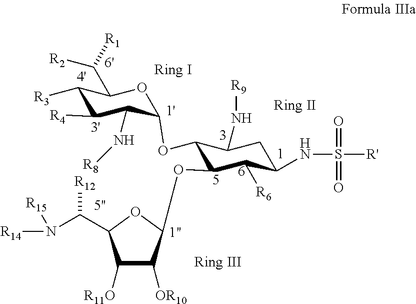
wherein:
R₁—R₄, R₆, R₈ and R₉ are as defined for Formula I or Formula Ia;
R₁₀, R₁₁, R₁₂, R₁₄ and R₁₅ are as defined for Formula III in claim 22; and
R′ is selected from hydrogen, a substituted or unsubstituted alkyl, a substituted or unsubstituted cycloalkyl, a substituted or unsubstituted alkaryl, and a substituted or unsubstituted aryl.

24. The compound of claim 23, wherein R₂ is ORx, and Rx is selected from hydrogen and a substituted or unsubstituted alkyl.

25. The compound of claim 24, wherein R₃ is ORy, and Ry is selected from hydrogen and a substituted or unsubstituted alkyl.

26-28. (canceled)

29. The compound of claim 23, wherein R₈ and R₉ are each hydrogen.

30. The compound of claim 23, wherein R₁₀, R₁₁, R₁₂, R₁₄ and R₁₅ are each hydrogen.

31. The compound of claim 23, wherein R₁₀, R₁₁, R₁₄ and R₁₅ are each hydrogen and Rig is selected from the group consisting of a substituted or unsubstituted alkyl, a substituted or unsubstituted cycloalkyl, and a substituted or unsubstituted aryl.

32. (canceled)

33. (canceled)

34. The compound of claim23, selected from the group consisting of:

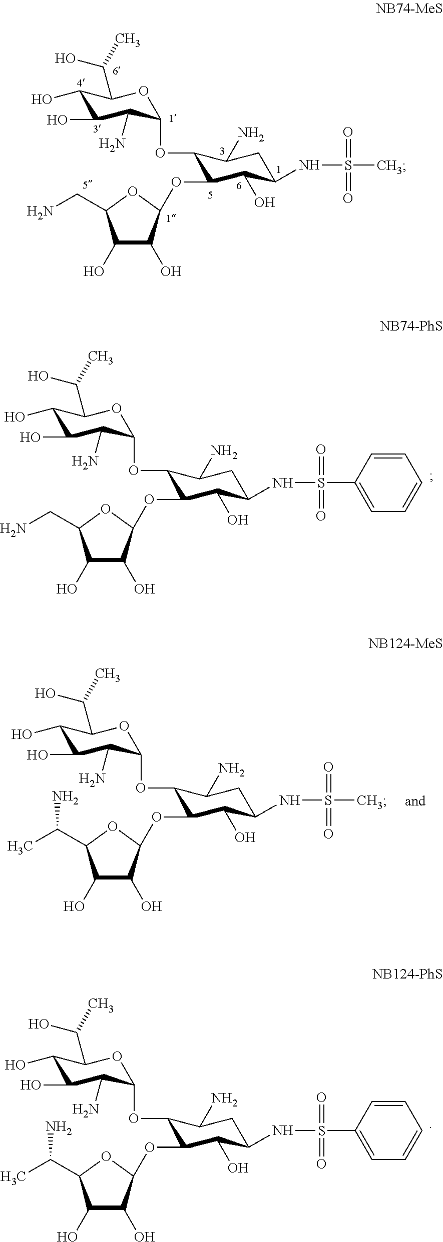

35-48. (canceled)

49. A pharmaceutical composition comprising the compound claim 1 and a pharmaceutically acceptable carrier.

50. The pharmaceutical composition of claim 49, for use in the treatment of a genetic disorder associated with a premature stop codon mutation.

51. (canceled)

52. The pharmaceutical composition of claim 49, for use in increasing an expression level of a gene having a stop codon mutation.
